(19) United States
(12) Patent Application Publication
(10) Pub. No.: US 2021/0395281 A1
(43) Pub. Date: Dec. 23, 2021

(54) MACROCYCLIC CHELATES AND USES THEREOF

(71) Applicant: Fusion Pharmaceuticals Inc., Hamilton (CA)

(72) Inventors: Stuart J. Mahoney, Brantford (CA); Michael B. Johansen, Cambridge (CA); Matthew D. Moran, Mississauga (CA); Melissa Chassé, Toronto (CA); Ryan W. Simms, Toronto (CA); Eric S. Burak, Cambridge (CA); John F. Valliant, Ancaster (CA)

(21) Appl. No.: 17/272,129

(22) PCT Filed: Jan. 8, 2021
(86) PCT No.: PCT/US2021/012697

§ 371 Date: Feb. 26, 2021

Related U.S. Application Data
(60) Provisional application No. 62/959,665, filed on Jan. 10, 2020.

(51) Int. Cl. C07F 9/6558; C07D 401/12; C07D 401/14; A61K 47/68; A61K 51/04; A61K 51/10

(52) U.S. Cl. CPC C07F 9/65583; C07D 401/12; C07D 401/14; A61K 51/103; A61K 51/0482; A61K 51/1093; A61K 47/6849

(57) ABSTRACT

The present disclosure relates to macrocyclic chelates including a macrocyclic chelating moiety of a metal complex thereof, a bifunctional linker, and a therapeutic or targeting moiety. Also disclosed are methods for preparation of the same, and use thereof.

### RELATED APPLICATION

The present application is the national stage of International (PCT) Patent Application Serial Number PCT/US2021/012697, filed Jan. 8, 2021, which claims priority to U.S. Provisional Patent Application No. 62/959,665 filed on Jan. 10, 2020; the entire contents of each of which are hereby incorporated by reference for all purposes.

### BACKGROUND

Radioconjugates, or radiolabelled targeting moieties, are widely used in theranostic applications. They typically contain a chelate capable of complexing a radionuclide, a linker, and a targeting moiety or cross-linking group. Radioconjugates are generally prepared by using a bifunctional chelator to append a radiolabel to a biological molecule while maintaining target affinity.

One of the main challenges associated with radioconjugates remains in identifying a chelate structure to complex desirable theranostic metal pairs such as zirconium (Zr) and actinium (Ac) that have distinct atomic properties. For example, Zr and Ac have different sizes, with their ionic radius being 0.59 Å and 1.12 Å, respectively (Acta Crystallogr. Sect. A 1976, 32, 751-767), and have different charges of 4+ and 3+, respectively. Further, currently known radioconjugates often lack sufficient in vivo stability, which limit their medical use. In addition, certain chelates require elevated thermal conditions for the radiolabeling process that are not compatible with having a targeting moiety (e.g., the elevated temperature would damage the structural integrity of an antibody targeting moiety) or cross-linking group pre-conjugated with a bifunctional chelator, which presents another factor limiting their use in relevant fields.

There is a need to develop new chelates that form stable complexes of both imaging-suitable metals (e.g., ⁸⁹Zr) and therapy-suitable metals (e.g., ²²⁵Ac) under mild conditions for theranostic applications.

### SUMMARY

The present invention relates to macrocyclic chelates that unexpectedly form, under mild conditions, stable complexes with both ⁸⁹Zr for imaging (e.g., Positron Emission Tomography or PET) and ²²⁵Ac for therapy (e.g., cancer treatment).

One aspect of this invention features certain compounds having the structure of formula (I) shown below, or metal complexes thereof, or pharmaceutically acceptable salts thereof:

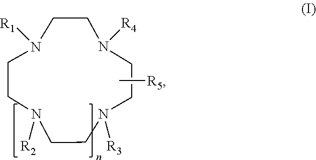

wherein

R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and

n is an integer of 0-3,

wherein

L is optionally substituted C₁₋₃ alkylene;

U is optionally substituted carboxylic acid or optionally substituted phosphonic acid;

W is a donating moiety capable of coordinating to a radiometal, in which the donating moiety is an optionally substituted hydroxypyridinone or a moiety selected from the group consisting of

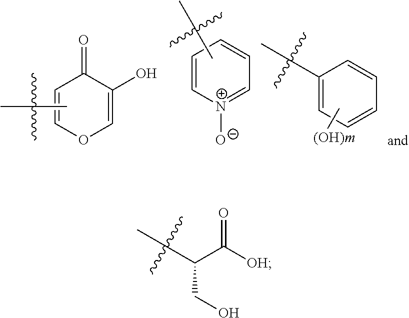

m is an integer of 1-3; and

X is -L¹-Z₁-L²-N(R)—(C═O)—, in which R is H, optionally substituted alkyl, optionally substituted heteroalkyl, or -L³-Z₂-B,

wherein

L¹ and L² each are, independently, bond, optionally substituted C₁-C₆ alkylene or optionally substituted C₁-C₆ heteroalkylene;

L³ is optionally substituted C₁-C₅₀ alkylene, or optionally substituted C₁-C₅₀ heteroalkylene, or C₅-C₂₀ polyethylene glycol;

Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;

Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and

B is a therapeutic moiety, a targeting moiety, or cross-linking group.

In some embodiments, W is an optionally substituted hydroxypyridinone, having one of the structures shown below:

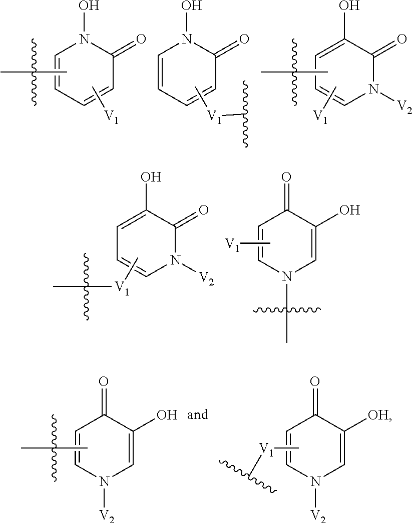

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

Another aspect of this invention features certain compounds having the structure of formula (I) shown below, or metal complexes thereof, or pharmaceutically acceptable salts thereof:

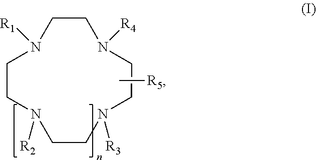

wherein

R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and

n is an integer of 0-3, when n is 0 and R₅ is H, R₁, R₃, and R₄ are not all equal to

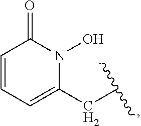

wherein

L is C═O or —CH(R)—, in which R is H, optionally substituted alkyl, optionally substituted heteroalkyl, or -L¹-Z₁-L²-Z₂-B;

U is optionally substituted aryl, optionally substituted heteroaryl, optionally substituted carboxylic acid, or optionally substituted phosphonic acid; or -L-U is -L¹-Z₁-L²-Z₂-B;

at least one of R₁-R₃ has U as optionally substituted heteroaryl;

X is C═O or optionally substituted C₁-C₃ alkylene; and

W is a donating moiety capable of coordinating to a radiometal, wherein the donating moiety is an optionally substituted hydroxypyridinone having the structure selected from the group consisting of

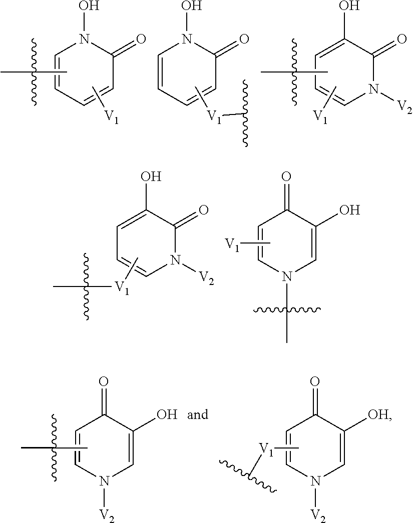

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl,
wherein

L¹ is bond, optionally substituted C₁-C₆ alkylene, or optionally substituted C₁-C₆ heteroalkylene;

Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;

L² is optionally substituted C₁-C₅₀ alkylene, or optionally substituted C₁-C₅₀ heteroalkylene, or C₅-C₂₀ polyethylene glycol;

Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and

B is a therapeutic moiety, a targeting moiety, or cross-linking group.

A further aspect of this invention features certain compounds having the structure of formula (II) shown below, or metal complexes thereof, or pharmaceutically acceptable salts thereof:

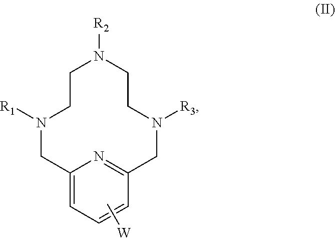

wherein

R₁, R₂, and R₃ each are, independently, -L-U, and W is H or -L¹-Z₁-L²-Z₂-B,

wherein

L is C═O or —CH(R)—, in which R is H, optionally substituted alkyl, optionally substituted heteroalkyl, or -L¹-Z₁-L²-Z₂-B;

U is optionally substituted aryl, optionally substituted heteroaryl, optionally substituted carboxylic acid, or optionally substituted phosphonic acid; or -L-U is -L¹-Z₁-L²-Z₂-B;

at least one of R₁-R₃ has U as optionally substituted heteroaryl;

wherein

L¹ is bond, optionally substituted C₁-C₆ alkylene, or optionally substituted C₁-C₆ heteroalkylene;

Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;

L² is optionally substituted C₁-C₅₀ alkylene, or optionally substituted C₁-C₅₀ heteroalkylene, or C₅-C₂₀ polyethylene glycol;

Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and

B is a therapeutic moiety, a targeting moiety, or cross-linking group.

In some embodiments, the compounds described above comprise variable B as a therapeutic moiety or targeting moiety. The therapeutic moiety or targeting moiety can be an antibody, or an antigen-binding fragment thereof.

In some embodiments, the antibody, or an antigen-binding fragment thereof, specifically binds insulin-like growth factor-1 receptor (IGF-1R).

In some embodiments, the compounds described above comprise variable B as a cross-linking group. The cross-linking group can be selected from an amino-reactive cross-linking group, a methionine-reactive cross-linking group, and a thiol-reactive cross-linking group.

In some embodiments, the cross-linking group comprises a moiety selected from an activated ester, an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, and oxaziridine. Each of these moieties refers to a chemical group commonly used in the field and known to a skilled artisan. For example, the activated ester can be a hydroxysuccinimide ester, 2,3,5,6-tetrafluorophenol ester, 2,6-dichlorophenol ester or a 4-nitrophenol ester.

In some embodiments, the compounds comprise variable B as a cross-linking group selected from the group consisting of:

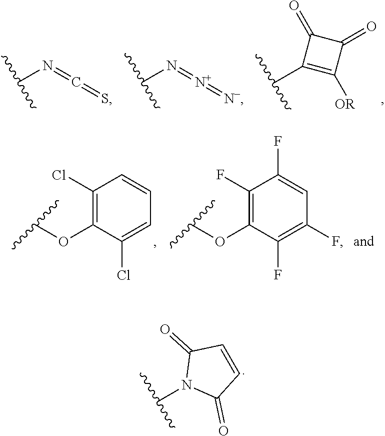

In some embodiments, the compounds described above comprise a metal complex that contains a metal selected from the group consisting of Bi, Pb, Y, Mn, Cr, Fe, Co, Zn, Ni, In, Ga, Cu, Re, Sm, a lanthanide, and an actinide.

In some embodiments, the compounds described above comprise a metal complex that contains a radionuclide selected from the group consisting of ⁸⁹Zr, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Ti, ²⁰³Pb, ²¹²Pb, ²¹²Bi, ²¹³Bi, ²²⁵Ac, ²²³Ra and ²²⁷Th.

In some embodiments, the compounds described above comprise a radionuclide of ⁸⁹Zr, ¹¹¹In, or ²²⁵Ac.

In still another aspect, the present invention features a pharmaceutical composition comprising any of the foregoing compounds and a pharmaceutically acceptable excipient (interchangeably used with “pharmaceutically acceptable carrier”).

Further covered by this invention is a method of radiation treatment planning and/or radiation treatment, the method comprising administering to a subject in need thereof any of the foregoing compounds or pharmaceutical compositions.

Still within the scope of this invention is a method of treating an immunoregulatory abnormality in a subject in need thereof, the method comprising administering to said subject one of the foregoing compounds in an amount effective for treating said immunoregulatory abnormality (e.g., cancer).

In some embodiments, the invention features a method of detecting and/or treating cancer, the method comprising administering to a subject in need thereof a first dose of any of the foregoing compounds or pharmaceutical compositions in an amount effective for radiation treatment planning, followed by administering subsequent doses of any of the foregoing compounds or pharmaceutical compositions in a therapeutically effective amount.

In some embodiments, the compound or composition administered in the first dose and the compound or composition administered in the second dose, or subsequent doses are the same.

In some embodiments, the compound or composition administered in the first dose and the compound or composition administered in the second dose, or subsequent doses are different.

In some embodiments, the cancer is a solid tumor or hematologic (liquid) cancer.

In some embodiments, the cancer is breast cancer, non-small cell lung cancer, small cell lung cancer, pancreatic cancer, head and neck cancer, prostate cancer, colorectal cancer, sarcoma, adrenocortical carcinoma, neuroendocrine cancer, Ewing's Sarcoma, multiple myeloma, or acute myeloid leukemia.

The cancer in the treatment of this invention can be formed from cells selected from breast cancer cells, non-small cell lung cancer cells, small cell lung cancer cells, pancreatic cancer cells, head and neck cancer cells, prostate cancer cells, colorectal cancer cells, thyroid cancer cells, sarcoma cells, adrenocortical carcinoma cells, Ewing's Sarcoma cells, glioblastoma multiforme cells, liver cancer cells, neuroendocrine tumor cells, bladder cancer cells, gastric and gastroesophageal junction cancer cells, melanoma cells, multiple myeloma cells, and acute myeloid leukemia cells.

In some embodiments, the foregoing methods further include administering an antiproliferative agent, radiation sensitizer, or an immunoregulatory or immunomodulatory agent.

In some embodiments, any of the foregoing compounds or compositions thereof and an antiproliferative agent or radiation sensitizer are administered within 28 days (e.g., within 14, 7, 6, 5, 4, 3, 2, or 1 day(s)) of each other.

In some embodiments, any of the above-described compounds or compositions thereof and an immunoregulatory or immunomodulatory agent are administered within 90 days (e.g., within 80, 70, 60, 50, 40, 30, 20, 10, 5, 4, 3, 2, or 1 day(s)) of each other.

In another aspect, the invention features a method of making a radioconjugate (e.g., a radioimmunoconjugate described below), wherein the method includes the following steps: (a) conjugating a bifunctional chelate to a biological molecule, (b) purifying the conjugate produced by step (a), and (c) chelating one or more radionuclides (e.g., one or more 225Ac radionuclides) with the purified conjugate of step (b) at a temperature of less than 35° C. (e.g., 20-30° C.) to produce a radioconjugate (e.g., an actinium radioconjugate).

In another aspect, the invention features a method of making a radioconjugate (e.g., a radioimmunoconjugate described below), wherein the method includes the following steps: (a) complexing one of the radionuclides (e.g., ²²⁵Ac radionuclide) with the bifunctional chelate, (b) optionally, purifying the radiolabeled bifunctional chelate produced by step (a), (c) conjugating the radiolabeled bifunctional chelate to a biological molecule to produce a radioconjugate (e.g., an actinium radioconjugate), and (d) optionally, purifying the radiolabeled antibody-conjugate product.

In some embodiments, the radioconjugate is a radioimmunoconjugate (e.g., any of the radioimmunoconjugates described herein).

In some embodiments, the temperature of the reaction mixture of conjugation step (c) is 20-34° C. (e.g., 21° C., 22° C., 23° C., 24° C., 25° C., 26° C., 27° C., 28° C., 29° C., 30° C., 31° C., 32° C., 33° C., or 34° C.).

In some embodiments, the pH of the reaction mixture of conjugation step (a) is 5.0-10.0 (e.g., 5.0-6.0, 6.0-7.0, 7.0-8.0, 8.0-9.0, or 9.0-10.0) In some embodiments, the pH of the reaction mixture of conjugation step (a) is less than 6.4 (e.g., 6.3, 6.2, 6.1, 6.0, 5.9, or 5.8 or less).

In some embodiments, the pH of the reaction mixture of chelation step (c) is between 5.5 and 7.0 (e.g., 5.5-6.0, 6.0-6.5, or 6.5-7.0) In some embodiments, the pH of the reaction mixture of chelating step (c) is less than 5.5 (e.g., 5.4, 5.3, 5.2, 5.1, or 5.0 or less) or more than 7.0 (e.g., 7.1, 7.2, 7.3, 7.4, 7.5 or more).

### Definitions

As used herein, the term “alkyl” or “alkylene” refers to a saturated, linear or branched hydrocarbon moiety, such as methyl, methylene, ethyl, ethylene, propyl, propylene, butyl, butylenes, pentyl, pentylene, hexyl, hexylene, heptyl, heptylene, octyl, octylene, nonyl, nonylene, decyl, decylene, undecyl, undecylene, dodecyl, dodecylene, tridecyl, tridecylene, tetradecyl, tetradecylene, pentadecyl, pentadecylene, hexadecyl, hexadecylene, heptadecyl, heptadecylene, octadecyl, octadecylene, nonadecyl, nonadecylene, icosyl, icosylene, triacontyl, and triacotylene.

As used herein, the term “heteroalkyl” or “heteroalkylene” refers to an aliphatic moiety (e.g., alkyl or alkylene) containing at least one heteroatom selected from N, O, P, B, S, Si, Sb, Al, Sn, As, Se, and Ge. Examples of “heteroalkyl” or “heteroalkylene” include, but are not limited to, the following moieties:

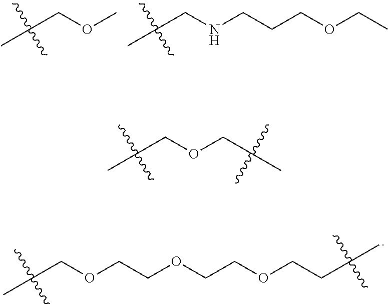

As used herein, the term “aryl” or “arylene” herein refers to a C₆ monocyclic, C₁₀ bicyclic, C₁₄ tricyclic, C₂₀ tetracyclic, or C₂₄ pentacyclic aromatic ring system. Examples of aryl or arylene groups include phenyl, phenylene, naphthyl, naphthylene, anthracenyl, anthracenylene, pyrenyl, and pyrenylene.

As used herein, the term “heteroaryl” or “heteroarylene” herein refers to an aromatic 5-8 membered monocyclic, 8-12 membered bicyclic, 11-14 membered tricyclic, and 15-20 membered tetracyclic ring system having one or more heteroatoms (such as O, N, S, or Se). Examples of heteroaryl or heteroarylene groups include furyl, furylene, fluorenyl, fluorenylene, pyrrolyl, pyrrolylene, thienyl, thienylene, oxazolyl, oxazolylene, imidazolyl, imidazolylene, benzimidazolyl, benzimidazolylene, thiazolyl, thiazolylene, pyridyl, pyridylene, pyrimidinyl, pyrimidinylene, quinazolinyl, quinazolinylene, quinolinyl, quinolinylene, isoquinolyl, isoquinolylene, indolyl, and indolylene.

Unless specified otherwise, alkyl, alkylene, heteroalkyl, heteroalkylene, aryl, arylene, heteroaryl, and heteroarylene mentioned herein include both substituted and unsubstituted moieties. Possible substituents on alkyl, alkylene, heteroalkyl, heteroalkylene, aryl, arylene, heteroaryl, and heteroarylene include, but are not limited to, C₁-C₁₀ alkyl, C₂-C₁₀ alkenyl, C₂-C₁₀ alkynyl, C₁-C₂M alkoxy, C₃-C₂₀ cycloalkyl, C₃-C₂₀ cycloalkenyl, C₃-C₂₀ heterocycloalkyl, C₃-C₂₀ heterocycloalkenyl, C₁-C₁₀ alkoxy, aryl, aryloxy, heteroaryl, heteroaryloxy, amino, C₁-C₁₀ alkylamino, C₂-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, oxo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. Each of these groups or moieties refers to a substituent commonly used in the field and known to a skilled artisan. Further, cycloalkyl, cycloalkylene, cycloalkenyl, cycloalkenylene, heterocycloalkyl, heterocycloalkylene, heterocycloalkenyl, heterocycloalkenylene, aryl, and heteroaryl can also be fused with each other.

For example, certain compounds of formula (I) have R₁, R₂, and R₃ each being, independently, -L-U, in which L is C═O or —CH(R)— and U is optionally substituted heteroaryl, wherein the optionally substituted heteroaryl is an optionally substituted hydroxypyridinone, having one of the structures shown below:

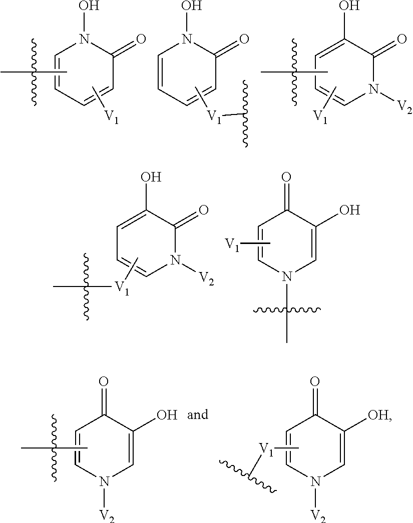

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

For example, certain compounds of formula (I) have R₁, R₂, R₃ and R₄ each being, independently, -L-U, in which L is C═O or —CH(R)— and U is optionally substituted aryl, optionally substituted heteroaryl, or optionally substituted carboxylic acid, or optionally substituted phosphonic acid, wherein R is optionally substituted heteroalkyl (substitution of carbon with oxo) selected from:

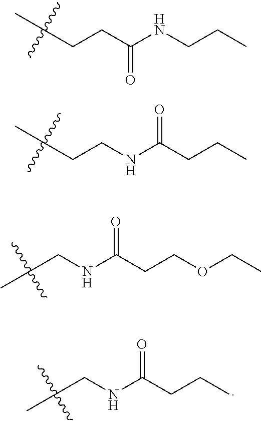

As used herein, the term “optionally substituted carboxylic acid” refers to a carboxylic acid or a derivative thereof, which can include an amide derived from the corresponding carboxylic acid. For example, U can be an amide as shown below:

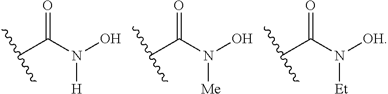 [SMILES: *C(=O)NO ; *C(=O)N(C)O ; *C(=O)N(O)CC]

As used herein, the term “optionally substituted phosphonic acid” refers to a phosphonic acid or a derivative thereof, which can include a phosphoramide derived from the corresponding phosphonic acid. For example, U can be a phosphoramide as shown below:

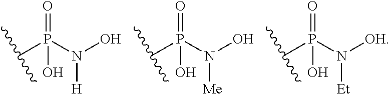 [SMILES: *P(=O)(O)NO ; *P(=O)(O)N(C)O ; *P(=O)(O)N(O)CC]

As used herein, the term “optionally substituted C₁-C₆ alkylene” refers to C₁-C₆ alkylene or a derivative thereof, which can include a C₁-C₆ alkylene group having one or more carbons substituted with oxo. Examples of substituted C₁-C₆ alkylene include, but are not limited to, the following moieties:

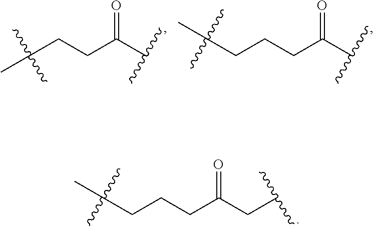 [SMILES: *CCC(*)=O ; *CCCC(*)=O ; *CCCC(=O)C*]

As used herein, the term “optionally substituted C₁-C₆ heteroalkylene” refers to C₁-C₆ heteroalkylene or a derivative thereof, which can include a C₁-C₆ heteroalkylene group having one or more carbons substituted with oxo. Examples of substituted C₁-C₆ heteroalkylene include, but are not limited to, the following moieties:

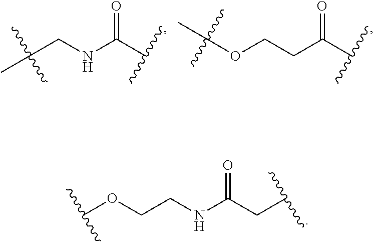 [SMILES: *CNC(*)=O ; *OCCC(*)=O ; *CC(=O)NCCO*]

As used herein, the term “optionally substituted C₁-C₅₀ heteroalkylene” refers to a C₁-C₅₀ heteroalkylene or a derivative thereof, which can include a heteroalkylene group having one or more carbons substituted with oxo. Examples of substituted C₁-C₅₀ heteroalkylene include, but are not limited to, the following moieties:

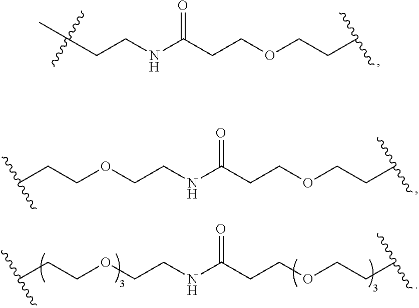

As used herein, the term “administered in combination” or “combined administration” means that two or more agents are administered to a subject at the same time or within an interval such that there may be an overlap of an effect of each agent on the patient. In some embodiments, they are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s), within 24 hours (e.g., 12, 6, 5, 4, 3, 2, or 1 hour(s), or within about 60, 30, 15, 10, 5, or 1 minute of one another. In some embodiments, the administrations of the agents are spaced sufficiently closely together such that a combinatorial (e.g., a synergistic) effect is achieved.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains and may include sequence elements found in the same polypeptide chain or in different polypeptide chains.

As used herein, “antigen-binding fragment” refers to a portion of an antibody that retains the binding characteristics of the parent antibody.

The terms “bifunctional chelate” or “bifunctional conjugate,” as used interchangeably herein, refer to a compound of formula (I) that contains a chelating group or metal complex thereof, a linker group, and an antibody or antigen-binding fragment thereof.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains an antibody or antigen-binding fragment thereof.

As used herein, the term “compound,” is meant to include all stereoisomers, geometric isomers, and tautomers of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ⁸⁹Zr, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²³Ra and ²²⁷Th). The terms radioactive nuclide, radioisotope, or radioactive isotope may also be used to describe a radionuclide. Radionuclides may be used as detection agents, as described above. In some embodiments, the radionuclide may be an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied.

The term “immunoconjugate,” as used herein, refers to a conjugate that includes a targeting moiety, such as an antibody (or antigen-binding fragment thereof). In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any radioconjugate that comprises a radioactive molecule attached to an immune substance, such as a monoclonal antibody, that can bind to cancer cells. A radioimmunoconjugate can carry radiation directly and specifically to cancer cells, thereby killing cancer cells without harming normal cells. Radioimmunoconjugates may also be used with imaging to help find cancer cells in the body.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt” herein represents those salts of the compounds described here that are suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The details of one or more embodiments of the disclosure are set forth in the description below. Other features, objects, and advantages of the disclosure will be apparent from the below drawing, description, and from the claims.

### BRIEF DESCRIPTION OF THE DRAWING

FIG. 1 depicts a biodistribution study of compound ⁸⁹Zr-Compound Y.

### DETAILED DESCRIPTION

Radiolabelled targeting moieties (also known as radioconjugates) are designed to target a protein or receptor that is upregulated in a disease state to deliver a radioactive payload to damage and kill cells of interest (radioimmunotherapy). The process of delivering such a payload, via radioactive decay, produces an alpha, beta, or gamma particle or Auger electron that can cause direct effects to DNA (such as single or double stranded DNA breaks) or indirect effects such as by-stander or crossfire effects.

Radioimmunoconjugates typically contain a biological targeting moiety (e.g, an antibody or antigen binding fragment thereof), a radioisotope, and a molecule that links the two. Conjugates are formed when a bifunctional chelate is appended to the biological targeting molecule so that structural alterations are minimal while maintaining target affinity. Once radiolabelled, the final radioimmunoconjugate is formed.

Bifunctional chelates structurally contain a chelate, the linker, and a targeting moiety (e.g., an antibody). When developing new bifunctional chelates, most efforts focus on the chelating portion of the molecule. Several examples of bifunctional chelates have been described with various cyclic and acyclic structures conjugated to a targeted moiety. See, e.g., Bioconjugate Chem. 2000, 11, 510-519; Bioconjugate Chem. 2012, 23, 1029-1039; Mol. Imaging Biol. 2011, 13, 215-221; and Bioconjugate Chem. 2002, 13, 110-115.

A commonly used chelate for in vivo ⁸⁹Zr PET imaging has been desferrioxamine (“DFO”), in part owing to its historical precedent as well as mild and efficient radiolabeling conditions. However, due to the stability issue, efforts have been greatly devoted to improving the in vivo stability of radioconjugates containing DFO chelate to reduce metal decomplexation. See, e.g., Chem. Comm. 2014, 50, 11523-11525; Chem. Comm. 2016, 52, 11889-11892.

The embodiments of the present disclosure relate to the structural identification of certain macrocyclic chelates that form radiometal complexes with high stability, e.g., the theranostic pair of ⁸⁹Zr and ²²⁵Ac, under mild radiolabeling conditions and as part of radioimmunoconjugates. The structural investigation was performed by modifying macrocyclic chelates in the linker region with a proximal donating group or by judicious substitution of the macrocyclic core including the use of hydroxypyridinones.

As discussed in the SUMMARY section above, one feature of the present disclosure features a first subset of compounds having the structure of formula (I) shown below, or metal complexes thereof, or pharmaceutically acceptable salts thereof:

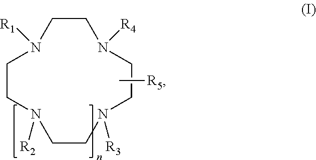

wherein

R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and

n is an integer of 0-3,

wherein

L is optionally substituted C₁₋₃ alkylene;

U is optionally substituted carboxylic acid or optionally substituted phosphonic acid;

W is a donating moiety capable of coordinating to a radiometal, in which the donating moiety is an optionally substituted hydroxypyridinone or a moiety selected from the group consisting of

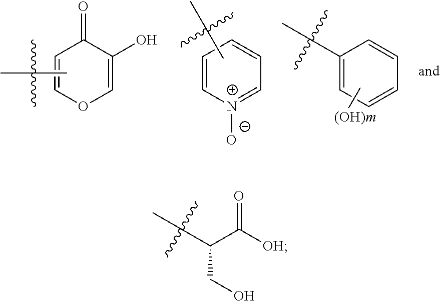

m is an integer of 1-3; and

X is -L¹-Z₁-L²-N(R)—(C═O)—, in which R is H, optionally substituted alkyl, optionally substituted heteroalkyl, or -L³-Z₂-B.

Referring to variable X, L¹ and L² each are, independently, bond, optionally substituted C₁-C₆ alkylene or optionally substituted C₁-C₆ heteroalkylene; L³ is optionally substituted C₁-C₅₀ alkylene, or optionally substituted C₁-C₅₀ heteroalkylene, or C₅-C₂₀ polyethylene glycol; Z₁ is C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl; Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and B is a therapeutic moiety, a targeting moiety, or a cross-linking group.

In some embodiments of the first subset, W is an optionally substituted hydroxypyridinone, having one of the structures shown below:

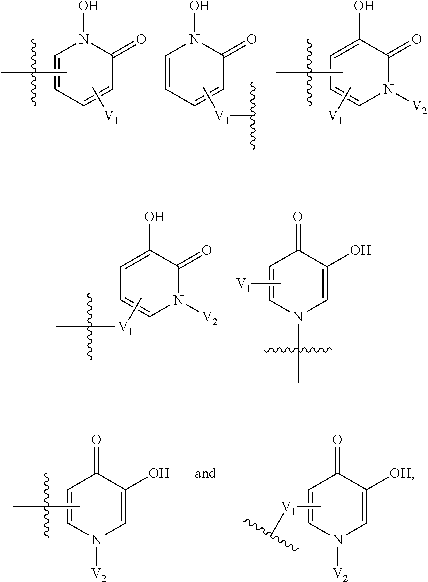

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl. For example, certain compounds feature that W is

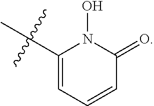 [SMILES: *c1cccc(=O)n1O]

In some embodiments of the first subset, R₁, R₂, and R₃ each are, independently, -L-U, in which L is optionally substituted C₁ alkyl (e.g., CH₂) and U is —CO₂H.

Also, certain compounds of the above embodiments have the structure of formula (I) that has n being 1.

In some embodiments of the first subset, X is -L¹-Z₁-L²-N(R)—(C═O)—, in which L¹ is

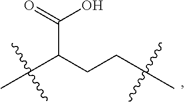 [SMILES: *CCC(*)C(=O)O]

### and R is H.

In some embodiments of the first subset, X is -L¹-Z₁-L²-N(R)—(C═O)—, in which L¹ is

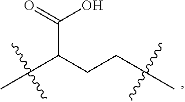 [SMILES: *CCC(*)C(=O)O]

and R is H; and each of R₁, R₂, and R₃ is -L-U, in which L is CH₂ and U is —CO₂H.

In some embodiments of the first subset, X is -L¹-Z₁-L²-N(R)—(C═O)—, in which L¹ is

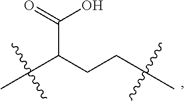 [SMILES: *CCC(*)C(=O)O]

### and R is H; and W is

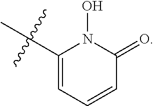 [SMILES: *c1cccc(=O)n1O]

In some embodiments of the first subset, X is -L¹-Z₁-L²-N(R)—(C═O)—, in which L¹ is

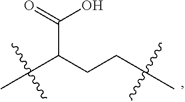 [SMILES: *CCC(*)C(=O)O]

and R is H; each of R₁, R₂, and R₃ is -L-U, in which L is CH₂ and U is —CO₂H; and W is

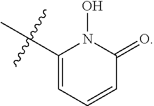 [SMILES: *c1cccc(=O)n1O]

In some embodiments of the first subset, X is -L¹-Z₁-L²-N(R)—(C═O)—, in which L¹ is

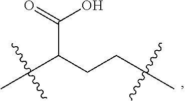 [SMILES: *CCC(*)C(=O)O]

and R is -L³-Z₂-B.

In some embodiments of the first subset, X is -L¹-Z₁-L²-N(R)—(C═O)—, in which L¹ is

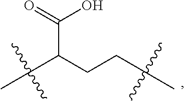 [SMILES: *CCC(*)C(=O)O]

and R is -L³-Z₂-B, in which L³ is C₅-C₂₀ polyethylene glycol and Z₂ is —NR′—(C═O)—R″, R′ being H and R″ being arylene.

In some embodiments of the first subset, X is -L¹-Z₁-L²-N(R)—(C═O)—, in which L¹ is

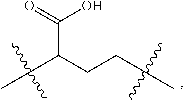 [SMILES: *CCC(*)C(=O)O]

and R is -L³-Z₂-B; and each of R₁, R₂, and R₃ is -L-U, in which L is CH₂ and U is —CO₂H.

In some embodiments of the first subset, X is -L¹-Z₁-L²-N(R)—(C═O)—, in which L¹ is

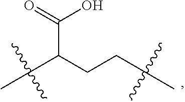 [SMILES: *CCC(*)C(=O)O]

and R is -L³-Z₂-B; and W is

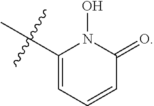 [SMILES: *c1cccc(=O)n1O]

In some embodiments of the first subset, X is -L¹-Z₁-L²-N(R)—(C═O)—, in which L¹ is

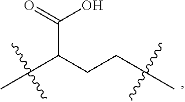 [SMILES: *CCC(*)C(=O)O]

and R is -L³-Z₂-B; W is

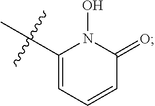 [SMILES: *c1cccc(=O)n1O]

each of R₁, R₂, and R₃ is -L-U, in which L is CH₂ and U is —CO₂H; L³ is C₅-C₂₀ polyethylene glycol; and Z₂ is —NR′—(C═O)—R″, R′ being H and R″ being arylene.

In some embodiments of the first subset, X is -L¹-Z₁-L²-N(R)—(C═O)—, in which L¹ is

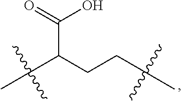 [SMILES: *CCC(*)C(=O)O]

and R is -L³-Z₂-B, in which B is a therapeutic moiety or targeting moiety.

In some embodiments of the first subset, X is -L¹-Z₁-L²-N(R)—(C═O)—, in which L¹ is

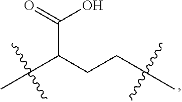 [SMILES: *CCC(*)C(=O)O]

and R is -L³-Z₂-B, in which B is an antibody, or an antigen-binding fragment thereof. For example, the antibody, or an antigen-binding fragment thereof, specifically binds insulin-like growth factor-1 receptor (IGF-1R).

In some embodiments of the first subset, X is -L¹-Z₁-L²-N(R)—(C═O)—, in which L¹ is

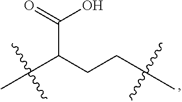 [SMILES: *CCC(*)C(=O)O]

and R is -L³-Z₂-B, in which B is a cross-linking group selected from the group consisting of an amino-reactive cross-linking group, a methionine-reactive cross-linking group, and a thiol-reactive cross-linking group. In some embodiments, the cross-linking group comprises an activated ester, an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine, in which the activated ester can be a hydroxysuccinimide ester, 2,3,5,6-tetrafluorophenol ester, 2,6-dichlorophenol ester or 4-nitrophenol ester.

In some embodiments of the first subset, B is a cross-linking group selected from the group consisting of:

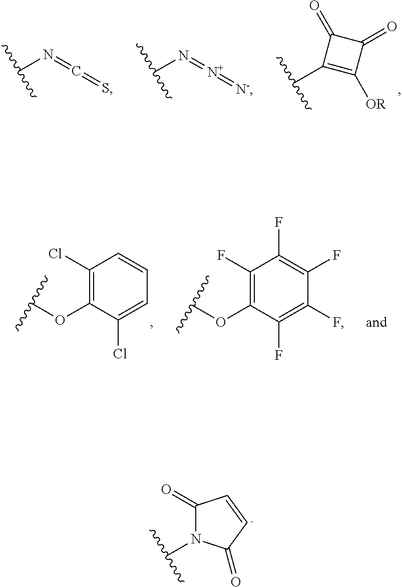

Another aspect of this invention features a second subset of compounds having the structure of formula (I) shown below, or metal complexes thereof, or pharmaceutically acceptable salts thereof:

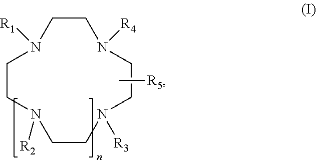

wherein

R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and

n is an integer of 0-3, when n is 0 and R₅ is H, R₁, R₃, and R₄ are not all equal to

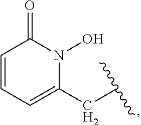

wherein

L is C═O or —CH(R)—, in which R is H, optionally substituted alkyl, optionally substituted heteroalkyl, or -L¹-Z₁-L²-Z₂-B;

U is optionally substituted aryl, optionally substituted heteroaryl, optionally substituted carboxylic acid, or optionally substituted phosphonic acid; or -L-U is -L¹-Z₁-L²-Z₂-B;

at least one of R₁-R₃ has U as optionally substituted heteroaryl;

X is C═O or optionally substituted C₁-C₃ alkylene; and

W is a donating moiety capable of coordinating to a radiometal, wherein the donating moiety is an optionally substituted hydroxypyridinone having the structure selected from the group consisting of

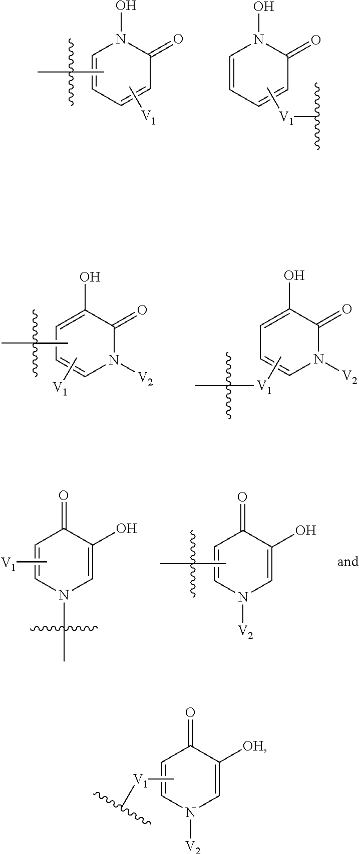

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

Referring to linker L being —CH(R)—, when R is -L¹-Z₁-L²-Z₂-B, each of variables L¹, Z₁, L², Z₂, and B is defined as follows:

L¹ is optionally bond, substituted C₁-C₆ alkylene, or optionally substituted C₁-C₆ heteroalkylene;

Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;

L² is optionally substituted C₁-C₅₀ alkylene, or optionally substituted C₁-C₅₀ heteroalkylene, or C₅-C₂₀ polyethylene glycol;

Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and

B is a therapeutic moiety, a targeting moiety, or cross-linking group.

In some embodiments of the second subset, W is an optionally substituted hydroxypyridinone having the structure:

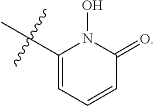 [SMILES: *c1cccc(=O)n1O]

In some embodiments of the second subset, X is C₁-C₃ alkylene.

In some embodiments of the second subset, W is an optionally substituted hydroxypyridinone having the structure:

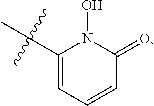 [SMILES: *c1cccc(=O)n1O]

### and X is CH₂.

In some embodiments of the second subset, compounds of formula (I) have variable n being 1.

In some embodiments of the second subset, W is an optionally substituted hydroxypyridinone having the structure:

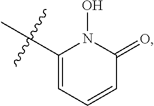 [SMILES: *c1cccc(=O)n1O]

### X is CH₂, and n is 1.

In some embodiments of the second subset, R₁, R₂, and R₃ each are, independently, -L-U, in which L is —CH(R)—, R being H.

In some embodiments of the second subset, R₁, R₂, and R₃ each are, independently, -L-U, in which L is —CH(R)—, R being H, and U is optionally substituted heteroaryl

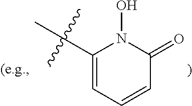 [SMILES: *c1cccc(=O)n1O]

or optionally substituted carboxylic acid (e.g., CO₂H or CO(NMeOH)).

In some embodiments of the second subset, R₁, R₂, and R₃ each are, independently, -L-U, in which L is —CH(R)—, R being H, and U is

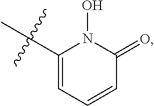 [SMILES: *c1cccc(=O)n1O]

CO₂H, or CO(NMeOH), and at least one of R₁-R₃ has U as

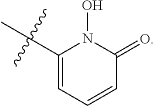 [SMILES: *c1cccc(=O)n1O]

In some embodiments of the second subset, R₁, R₂, and R₃ each are, independently, -L-U, in which L is —CH(R)—, R being H, and at least one of R₁-R₃ has U as

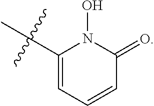 [SMILES: *c1cccc(=O)n1O]

In some embodiments of the second subset, R₁, R₂, and R₃ each are, independently, -L-U, in which L is —CH(R)—, R being H, and each of R₁-R₃ has U as

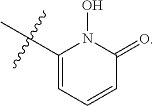 [SMILES: *c1cccc(=O)n1O]

In some embodiments of the second subset, R₁, R₂, and R₃ each are, independently, -L-U, in which L is —CH(R)—, R being H, and each of R₁-R₃ has U as

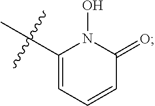 [SMILES: *c1cccc(=O)n1O]

### W is

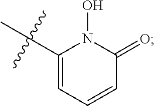 [SMILES: *c1cccc(=O)n1O]

### and X is CH₂.

In some embodiments of the second subset, X is C₁-C₃ alkylene and each of R₁-R₃ has U as

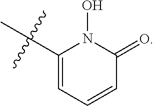 [SMILES: *c1cccc(=O)n1O]

In some embodiments of the second subset, each of R₁-R₃ has U as

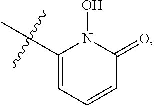 [SMILES: *c1cccc(=O)n1O]

### W is

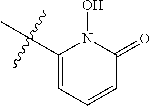 [SMILES: *c1cccc(=O)n1O]

### and X is CH₂.

In some embodiments of the second subset, R₁, R₂, and R₃ each are, independently, -L-U, in which L is —CH(R)—, R being -L¹-Z₁-L²-Z₂-B and L¹ being

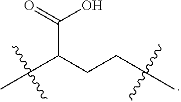 [SMILES: *CCC(*)C(=O)O]

In some embodiments of the second subset, R₁, R₂, and R₃ each are, independently, -L-U, in which L is —CH(R)—, R being -L¹-Z₁-L²-Z₂-B, wherein L¹ is

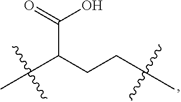 [SMILES: *CCC(*)C(=O)O]

L² is C₅-C₂₀ polyethylene glycol, and Z₂ is —NR′—(C═O)—R″, R′ being H and R″ being arylene.

In some embodiments of the second subset, R₁, R₂, and R₃ each are, independently, -L-U, in which L is —CH(R)—, R being -L¹-Z₁-L²-Z₂-B and L¹ being

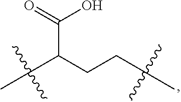 [SMILES: *CCC(*)C(=O)O]

at least one of R₁-R₃ has U as

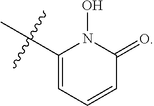 [SMILES: *c1cccc(=O)n1O]

In some embodiments of the second subset, R₁, R₂, and R₃ each are, independently, -L-U, in which L is —CH(R)—, R being -L¹-Z₁-L²-Z₂-B, wherein L¹ is

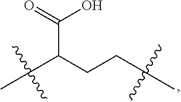 [SMILES: *CCC(*)C(=O)O]

and B is a therapeutic moiety or targeting moiety.

Typically, the therapeutic moiety or targeting moiety in this subset of compounds is an antibody, or an antigen-binding fragment thereof. For example, the antibody, or an antigen-binding fragment thereof, specifically binds IGF-1R.

In some embodiments of the second subset, R₁, R₂, and R₃ each are, independently, -L-U, in which L is —CH(R)—, R being -L¹-Z₁-L²-Z₂-B, wherein L¹ is

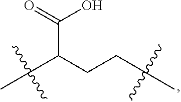 [SMILES: *CCC(*)C(=O)O]

and B is a cross-linking group selected from the group consisting of an amino-reactive cross-linking group, a methionine-reactive cross-linking group, and a thiol-reactive cross-linking group.

A further aspect of this invention features a third subset of compounds having the structure of formula (II) shown below, or metal complexes thereof, or pharmaceutically acceptable salts thereof:

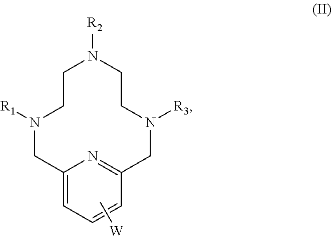

wherein

R₁, R₂, and R₃ each are, independently, -L-U, and W is H or -L¹-Z₁-L²-Z₂-B,

wherein

L is C═O or —CH(R)—, in which R is H, optionally substituted alkyl, optionally substituted heteroalkyl, or -L¹-Z₁-L²-Z₂-B;

U is optionally substituted aryl, optionally substituted heteroaryl, optionally substituted carboxylic acid, or optionally substituted phosphonic acid; or -L-U is -L¹-Z₁-L²-Z₂-B;

at least one of R₁-R₃ has U as optionally substituted heteroaryl;

wherein

L¹ is bond, optionally substituted C₁-C₆ alkylene, or optionally substituted C₁-C₆ heteroalkylene;

Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;

L² is optionally substituted C₁-C₅₀ alkylene, or optionally substituted C₁-C₅₀ heteroalkylene, or C₅-C₂₀ polyethylene glycol;

Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and

B is a therapeutic moiety, a targeting moiety, or cross-linking group.

In some embodiments, the compounds of formula (II) above feature that U is a donating moiety capable of coordinating to a radiometal, wherein the donating moiety is an optionally substituted hydroxypyridinone having the structure selected from the group consisting of

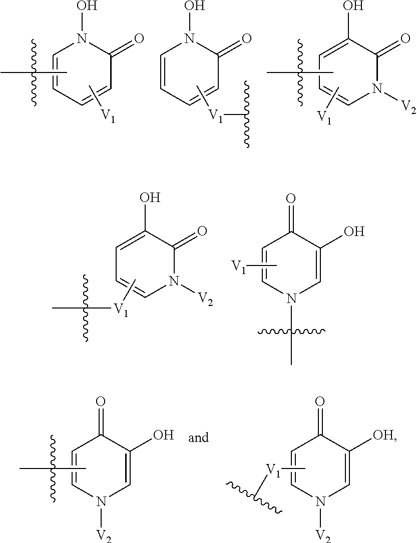

in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

Examples of the third subset of compounds of formula (II) include, but are not limited to, the following.

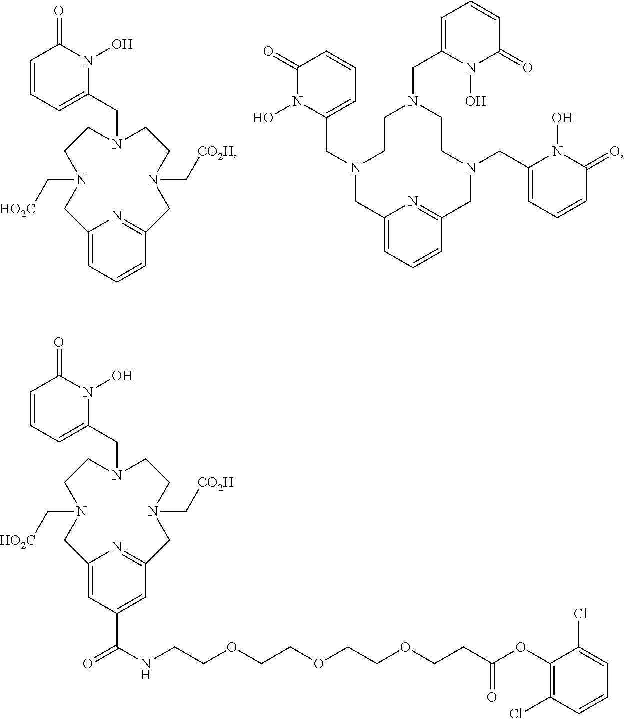 [SMILES: O=C(O)CN1CCN(Cc2cccc(=O)n2O)CCN(CC(=O)O)Cc2cccc(n2)C1 ; O=c1cccc(CN2CCN(Cc3cccc(=O)n3O)Cc3cccc(n3)CN(Cc3cccc(=O)n3O)CC2)n1O ; O=C(O)CN1CCN(Cc2cccc(=O)n2O)CCN(CC(=O)O)Cc2cc(C(=O)NCCOCCOCCOCCC(=O)Oc3c(Cl)cccc3Cl)cc(n2)C1]

Typically, the cross-linking group in any subset of the compounds described above comprises an activated ester, an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine, in which the activated ester can be a hydroxysuccinimide ester, 2,3,5,6-tetrafluorophenol ester, 2,6-dichlorophenol ester or 4-nitrophenol ester. An exemplary cross-linking group is selected from the group consisting of:

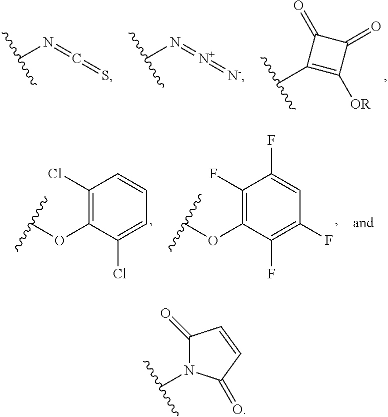

Through the embodiments described herein, bifunctional chelates, when attached to certain antibodies (e.g., IGF-1R), have been identified that achieve a reduction of total body radioactivity, thus minimizing toxicity, by enhancing in vivo stability of the radioimmunoconjugates. When taken in whole, these embodiments achieve the desired properties of radioimmunoconjugates by reducing the radioactivity in the human body while maintaining on-target activity.

### Therapeutic Moieties and Targeting Moieties

Therapeutic or targeting moieties include any molecule or any part of a molecule that confers a therapeutic benefit. In some embodiments, the therapeutic moiety is a protein or polypeptide, e.g., an antibody, an antigen-binding fragment thereof. In some embodiments, the therapeutic moiety is a small molecule. Targeting moieties include any molecule or any part of a molecule that binds to a given target.

### Antibodies

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991), or any of the molecules described below.

“Avimer” relates to a multimeric binding protein or peptide engineered using, for example, in vitro exon shuffling and phage display. Multiple binding domains are linked, resulting in greater affinity and specificity compared to single epitope immunoglobin domains.

“Nanobodies” are antibody fragments consisting of a single monomeric variable antibody domain. Nanobodies may also be referred to as single-domain antibodies. Like antibodies, nanobodies bind selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display). “Affibodies” are polypeptides or proteins engineered to bind to a specific antigen. As such, affibodies may be considered to mimic certain functions of antibodies. Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g., insulin, fibrinogen, transferrin, tumor necrosis factor-α, IL-8, gp120, CD28, human serum albumin, IgA, IgE, IgM, HER2 and EGFR) have been generated, demonstrating affinities (Kd) in the μM to pM range. “Diabodies” are antibody fragments with two antigen-binding sites that may be bivalent or bispecific. See for example Hudson et al., (2003). Single-chain antibodies are antibody fragments comprising all or a portion of the heavy chain variable domain or all, or a portion of the light chain variable domain of an antibody. Antibody fragments can be made by various techniques including but not limited to proteolytic digestion of an intact antibody as well as production by recombinant hosts (e.g., E. coli or phage) as described herein.

In certain embodiments, the antibody or antigen-binding fragment thereof is a multispecific, e.g. bispecific. Multispecific antibodies (or antigen-binding fragments thereof) include monoclonal antibodies (or antigen-binding fragments thereof) that have binding specificities for at least two different sites.

In certain embodiments, amino acid sequence variants of antibodies or antigen-binding fragments thereof are contemplated; e.g., variants that bind to IGF-1R. For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A reference polypeptide described herein can include a target-binding domain that binds to a target of interest (e.g., binds to an antigen). For example, a polypeptide, such as an antibody, can bind to a transmembrane polypeptide (e.g., receptor) or ligand (e.g., a growth factor). Exemplary molecular targets (e.g., antigens) for polypeptides described herein (e.g., antibodies) include CD proteins such as CD2, CD3, CD4, CD8, CD11, CD19, CD20, CD22, CD25, CD33, CD34, CD40, CD52; members of the ErbB receptor family such as the EGF receptor (EGFR, HER1, ErbB1), HER2 (ErbB2), HER3 (ErbB3) or HER4 (ErbB4) receptor; macrophage receptors such as CRIg; tumor necrosis factors such as TNFα or TRAIL/Apo-2; cell adhesion molecules such as LFA-1, Mac1, p150, 95, VLA-4, ICAM-1, VCAM and αvβ3 integrin including either α or β subunits thereof (e.g., anti-CD11a, anti-CD18 or anti-CD11b antibodies); growth factors and receptors such as EGF, FGFR (e.g., FGFR3) and VEGF; IgE; cytokines such as IL1; cytokine receptors such as IL2 receptor; blood group antigens; flk2/flt3 receptor; obesity (OB) receptor; mpl receptor; CTLA-4; protein C; neutropilins; ephrins and receptors; netrins and receptors; slit and receptors; chemokines and chemokine receptors such as CCL5, CCR4, CCR5; amyloid beta; complement factors, such as complement factor D; lipoproteins, such as oxidized LDL (oxLDL); lymphotoxins, such as lymphotoxin alpha (LTa). Other molecular targets include Tweak, B7RP-1, proprotein convertase subtilisin/kexin type 9 (PCSK9), sclerostin, c-kit, Tie-2, c-fins, and anti-M1.

### Modified Polypeptides

The polypeptides of the invention may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to IGF-1R). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of any of the polypeptides of the invention can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1 below. If such substitutions result in a change not desired, then other type of substitutions, denominated “exemplary substitutions” in Table 1 below, or as further described herein in reference to amino acid classes, are introduced and the products screened.

TABLE 1
Amino acid substitutions

| Original | Exemplary | Conservative |
|---|---|---|
| residue | substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, Norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

### Detection Agents

A detection agent is a molecule or atom which is administered conjugated to a polypeptide, e.g., an antibody or antigen-binding fragment thereof, and is useful in diagnosing a disease by locating the cells containing the antigen, radiation treatment planning, or treatment of a disease. Useful detection agents include, but are not limited to, radioisotopes, dyes (such as with the biotin-streptavidin complex), contrast agents, fluorescent compounds or molecules, luminescent agents, and enhancing agents (e.g., paramagnetic ions) for magnetic resonance imaging (MRI). In order to load a polypeptide component with a detection agent it may be necessary to react it with a reagent having a linker to which are attached the detection agent or multiple detection agents.

### Radioisotopes and Radionuclides

Radioisotopes and radionuclides known in the art for their utility as detection agents include, but are not limited to, ⁸⁹Zr, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²³Ra and ²²⁷Th.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 1990, 249, 1527-1533).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that include the above mention agents dissolved or suspended in an acceptable carrier, preferably an aqueous carrier, e.g., water, buffered water, saline, or PBS, among others. The compositions may contain pharmaceutically acceptable auxiliary substances as required to approximate physiological conditions, such as pH adjusting and buffering agents, tonicity adjusting agents, wetting agents, or detergents, among others. The invention also provides compositions for oral delivery, which may contain inert ingredients such as binders or fillers for the formulation of a unit dosage form, such as a tablet or a capsule. Furthermore, this invention provides compositions for local administration, which may contain inert ingredients such as solvents or emulsifiers for the formulation of a cream, an ointment, a gel, a paste, or an eye drop.

These compositions may be sterilized by conventional sterilization techniques or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 6 and 7, such as 6 to 6.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for radiation treatment planning, diagnostic, or therapeutic treatments. When administered for radiation treatment planning or diagnostic purposes, the conjugate is administered to a subject in a diagnostically effective dose and/or an amount effective to determine the therapeutically effective dose. In therapeutic applications, compositions are administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. The conjugates of the invention can be used for the treatment of cancer by administering to a subject a first dose of any of the foregoing conjugates or compositions in an amount effective for radiation treatment planning, followed by administering a second dose of any of the foregoing conjugates or compositions in a therapeutically effective amount.

Amounts effective for these uses may depend on the severity of the disease or condition and the weight and general state of the subject. The therapeutically effective amount of the compositions of the invention and used in the methods of this invention applied to mammals (e.g., humans) can be determined by the ordinarily skilled artisan with consideration of individual differences in age, weight, and the condition of the mammal. Because certain conjugates of the invention exhibit an enhanced ability to target cancer cells and residualize, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated and/or non-radiolabeled agent. The agents of the invention are administered to a subject (e.g., a mammal, such as a human) in an effective amount, which is an amount that produces a desirable result in a treated subject. Therapeutically effective amounts can also be determined empirically by those of skill in the art.

Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The conjugates of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever.

### EXAMPLES

### Example 1: Materials and General Methods

Actinium-225 (²²⁵Ac) was supplied by the U.S. Department of Energy Isotope Program in the Office of Science for Nuclear Physics. Lutetium-177 (¹⁷⁷Lu) was received from ITG Isotope Technologies Garching GmbH, and Zirconium-89 (⁸⁹Zr) was received from 3D Imaging.

MALDI-TOF-MS (positive ion) was used to determine the chelate-to-antibody ratio of immunoconjugates. Matrix-Assisted Laser Desorption/Ionization Time-of-Flight Mass Spectrometry (MALDI-TOF-MS) was performed using a MALDI Bruker Ultraflextreme Spectrometer. A saturated solution of sinapinic acid was prepared in TA30 solvent (30:70 [v/v]acetonitrile: 0.1% TFA in water). The samples were mixed in a 1:1 ratio with the matrix solution. A sample volume of 1 μL was spotted on the plate and a protein solution of BSA was used as an external standard.

Size exclusion chromatography (SEC) was performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWxl, 7.8×300 mm column.

SEC HPLC Elution Method 1: The isocratic SEC method had a flow rate=0.5 mL/min, with a mobile phase of 0.2 M potassium phosphate (pH 7), 0.25 M potassium chloride, 10% isopropanol, pH=7.

SEC HPLC Elution Method 2: The isocratic SEC method had a flow rate=1.0 mL/min, with a mobile phase of 0.022 M NaH₂PO₄, 0.047 M Na₂HPO₄, 0.60 M sodium chloride, 0.0038 M sodium azide, pH=7

RadioTLC was performed with Bioscan AR-2000 Imaging Scanner, carried out on iTLC-SG glass microfiber chromatography paper (Agilent Technologies, SGI0001) plates.

Radioactive preparative reverse phase HPLC was performed using a Waters system comprised of a Waters 1525 Binary HPLC pump, a Waters 2489 UV/Visible Detector (monitoring at 254 and 214 nm), a Bioscan Flow Count radiodetector (FC-3300) and Atlantis T3, 4.6×150 mm (5 μm) column, no guard; mobile phase A: H₂O (0.1% v/v TFA); mobile phase B: acetonitrile (0.1% v/v TFA); flow rate=1.5 mL/min; initial=100% A, 3 min=100% A, 13 min=75% A, 15 min=0% A.

Radioactive preparative SEC HPLC was performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWxl, 7.8×300 mm column. The isocratic SEC method had a flow rate=1.0 mL/min, with a mobile phase of 0.022 M NaH₂PO₄, 0.047 M Na₂HPO₄, 0.60 M sodium chloride, pH=7.

Analytical HPLC-MS was performed using a Waters Acquity HPLC-MS system comprised of a Waters Acquity Binary Solvent Manager, a Waters Acquity Sample Manager, a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 mm (1.7 μm) column. Preparative HPLC was performed using a Waters HPLC system comprised of a Waters 1525 Binary HPLC pump, a Waters 2489 UV/Visible Detector (monitoring at 254 nm and 214 nm) and a Waters XBridge Prep C18 19×100 mm (5 μm) column or Waters XBridge Prep Phenyl 19×100 mm (5 μm).

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; wavelength=214, 254 nm; initial=98% A, 3 min=98% A, 8 min=75% A, 10 min=0% A, 11 min=98% A, 12 min=98% A.

HPLC elution method 2: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; wavelength=214, 254 nm; initial=90% A, 8 min=0% A, 10 min=0% A, 11 min=90% A, 12 min=90% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; wavelength=214, 254 nm; initial=95% A, 8 min=75% A, 10 min=0% A, 11 min=95% A, 12 min=95% A.

### Example 2: Synthesis of 4-({2-[(1-Hydroxy-6-oxopyridin-2-yl)formamido]ethyl}carbamoyl)-2-[4,7,10-tris(carboxymethyl)-1,4,7,10-tetraazacyclododecan-1-yl]butanoic Acid (Compound A)

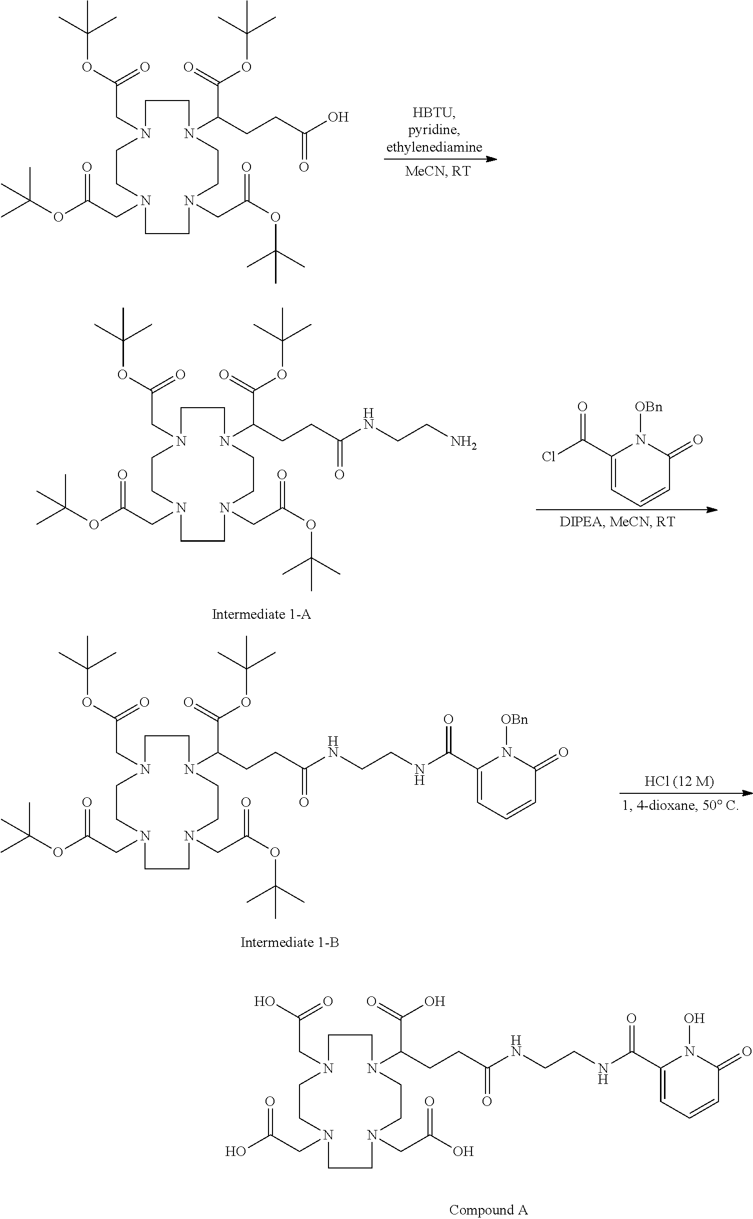

### Step 1: Synthesis of tert-Butyl-4-[(2-aminoethyl)carbamoyl]-2-{4,7,10-tris[2-(tert-butoxy)-2-oxoethyl]-1,4,7,10-tetraazacyclododecan-1-yl}butanoate (Intermediate 1-A)

To a 50 mL round bottom flask with a stir bar was added DOTA-GA(tBu)₄ (500 mg, 0.70 mmol, 1 equiv), HBTU (300 mg, 0.77 mmol, 1.1 equiv), anhydrous MeCN (30 mL) and lastly pyridine (2.94 mL, 36.3 mmol, 52 equiv). The reaction was stirred at room temperature for 30 min and then was drawn into a syringe and delivered by a syringe pump at a rate of 0.5 mL/min over 1 h into a 100 mL round bottom flask containing ethylenediamine (9.3 mL, 139 mmol, 200 equiv) and anhydrous MeCN (20 mL) stirring at room temperature. The reaction was monitored by HPLC-MS and upon completion was concentrated under vacuum and then purified on a preparative C18 HPLC column to afford Intermediate 1-A (435 mg, 64%) as a white/clear residue as the TFA salt.

### Step 2: Synthesis of tert-Butyl-4-[(2-{[1-(benzyloxy)-6-oxopyridin-2-yl]formamido}ethyl)carbamoyl]-2-{4,7,10-tris[2-(tert-butoxy)-2-oxoethyl]-1,4,7,10-tetraazacyclododecan-1-yl}butanoate (Intermediate 1-B)

To a 20 mL scintillation vial with a stir bar containing tert-butyl-4-[(2-aminoethyl)carbamoyl]-2-{4,7,10-tris[2-(tert-butoxy)-2-oxoethyl]-1,4,7,10-tetraazacyclododecan-1-yl}butanoate (Intermediate 1-A) TFA salt (125 mg, 0.13 mmol) was added anhydrous MeCN (4 mL), N,N-diisopropylethylamine (90 μL, 0.51 mmol) and lastly 1-(benzyloxy)-6-oxo-1,6-dihydropyridine-2-carbonyl chloride (J. Med. Chem. 2014, 57, 4849-486) (43 mg, 0.16 mmol, dissolved in 496 μL of anhydrous MeCN). The resulting solution was stirred at room temperature for 2 h and then monitored by HPLC-MS. Upon completion the reaction was worked up by concentration under vacuum and then purified on a preparative C18 HPLC column to afford Intermediate 1-B (133 mg, 86%) as a pale-yellow residue as the TFA salt.

### Step 3: Synthesis of 4-({2-[(1-Hydroxy-6-oxopyridin-2-yl)formamido]ethyl}carbamoyl)-2-[4,7,10-tris(carboxymethyl)-1,4,7,10-tetraazacyclododecan-1-yl]butanoic Acid (Compound A)

To a 20 mL scintillation vial containing tert-butyl-4-[(2-{[1-(benzyloxy)-6-oxopyridin-2-yl]formamido}ethyl)carbamoyl]-2-{4,7,10-tris[2-(tert-butoxy)-2-oxoethyl]-1,4,7,10-tetraazacyclododecan-1-yl}butanoate (Intermediate 1-B, 10 mg, 8.3 μmol) and a stir bar was added 1,4-dioxane (0.5 mL) and HCl (0.5 mL, 12 M, trace metal analysis grade). The resulting solution was capped and stirred in an oil bath at 50° C. and the reaction progress was monitored by HPLC-MS. Upon completion the reaction was worked up by concentration to dryness under an air stream and then purified on a preparative C18 HPLC column to afford Compound A (13.5 mg, quant.) as a white solid as the TFA salt following lyophilization. An aliquot was analyzed by HPLC-MS elution using elution method 1; retention time: 1.74 min; MS (positive ESI): found m/z 656.0 [M+H]⁺; C₂₇H₄₂N₇O₁₂ (calc. 656.3).

### Example 3: Synthesis of 4-({2-[1-(1-Hydroxy-6-oxopyridin-2-yl)-N-{2-[2-(2-{2-[(4-isothiocyanatophenyl)formamido]ethoxy}ethoxy)ethoxy]ethyl}formamido]ethyl}carbamoyl)-2-[4,7,10-tris(carboxymethyl)-1,4,7,10-tetraazacyclododecan-1-yl]butanoic Acid (Compound B)

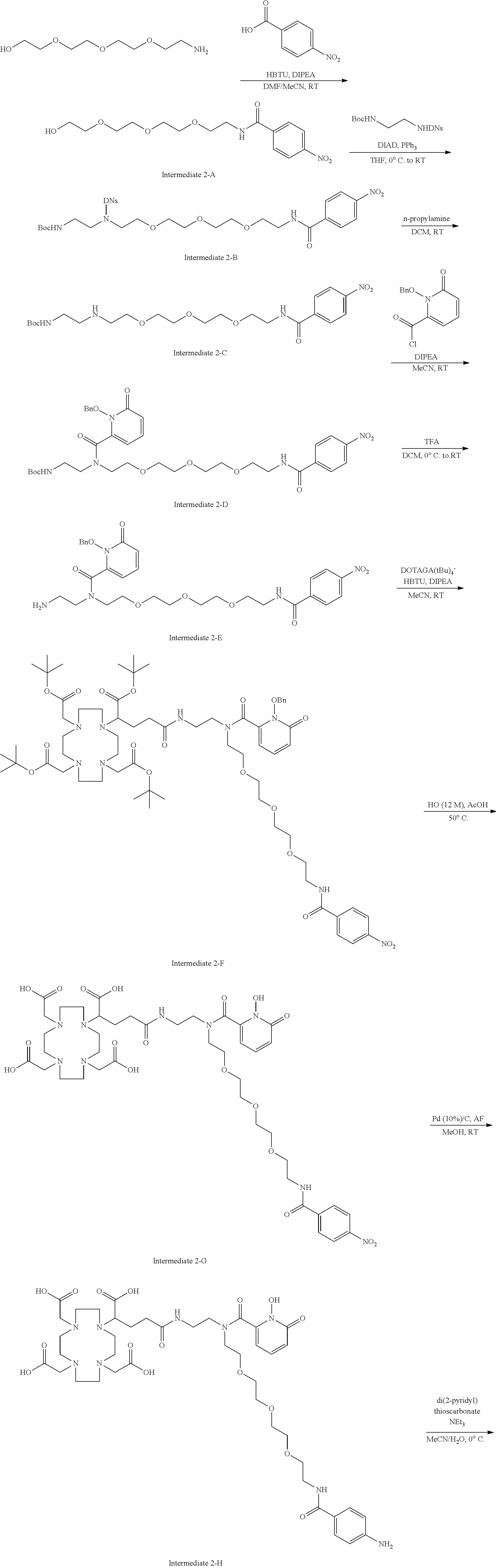

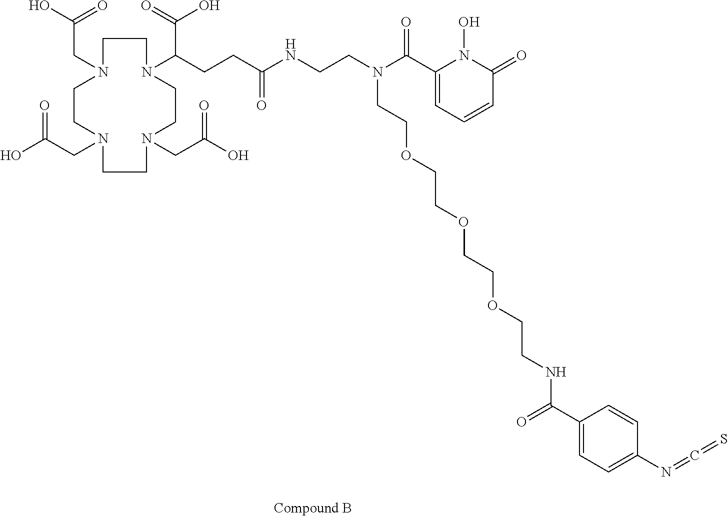 [SMILES: O=C(O)CN1CCN(CC(=O)O)CCN(C(CCC(=O)NCCN(CCOCCOCCOCCNC(=O)c2ccc(N=C=S)cc2)C(=O)c2cccc(=O)n2O)C(=O)O)CCN(CC(=O)O)CC1]

### Step 1: Synthesis of N-(2-{2-[2-(2-Hydroxyethoxy)ethoxy]ethoxy}ethyl)-4-nitrobenzamide (Intermediate 2-A)

To a 500 mL round bottom flask with stir bar containing 4-nitrobenzoic acid (2.00 g, 11.7 mmol) was added anhydrous DMF (40 mL) and anhydrous MeCN (20 mL), followed by DIPEA (4.00 mL, 22.7 mmol) and HBTU (4.99 g, 12.9 mmol). The resulting solution was stirred at room temperature for 1 h and then a solution of amino-PEG4-alcohol (2.54 g, 12.9 mmol) in anhydrous DMF (6 mL) was added dropwise over 30 min by syringe pump at a rate of 0.3 mL/min. The reaction progress was monitored by HPLC-MS and upon completion the reaction was concentrated to dryness under vacuum and then residual DMF was removed under an air stream to afford a brown oily residue. The crude residue was then dissolved in DCM (200 mL) and then washed successively with NaOH (1 M, 100 mL), HCl (1 M, 100 mL) and lastly brine (100 mL). The organic layer was then dried with sodium sulfate, decanted and concentrated under vacuum. The crude was then purified by silica gel column chromatography and eluted with the following steps: EtOAc to 300 MeOH/97% DCM (v/v) to 5% MeOH/95% DCM (v/v) to 10% MeOH/90% DCM (v/v) to MeOH). The product eluted in the later part of the elution from 10% MeOH/90% DCM (v/v) to MeOH. After concentration of the product containing fractions under vacuum obtained Intermediate 2-A (1.77 g, 32%, 71% purity) as a brown/orange oil.

### Step 2: Synthesis of tert-Butyl N-[2-(N-{2-[2-(2-{2-[(4-nitrophenyl)formamido]ethoxy}ethoxy)ethoxy]ethyl}2,4-dinitrobenzenesulfonamido)ethyl]carbamate (Intermediate 2-B)

A round bottomed flask was charged with N-(2-{2-[2-(2-hydroxyethoxy)ethoxy]ethoxy}ethyl)-4-nitrobenzamide (Intermediate 2-A, 1.45 g, 3.02 mmol, 71% purity), tert-butyl N-[2-(2,4-dinitrobenzenesulfonamido)ethyl]carbamate (1.53 g, 3.93 mmol), a stir bar, anhydrous THE (52 mL) and was then cooled in an ice bath at 0° C. DIAD (0.88 mL, 4.23 mmol) was then added dropwise manually over 5 min while the reaction stirred. Lastly, triphenylphosphine (1.12 g, 4.23 mmol) was added over approximately 2 min and the reaction was removed from the ice bath and stirred at room temperature. The reaction progress was monitored by HPLC-MS and was complete after 1 h. The reaction was worked up by concentrating under vacuum to obtain an orange oil. The crude was then purified by silica gel chromatography and eluted with the following steps: 50% EtOAc/50% Hexanes (v/v) to EtOAc to 10% MeOH/90% DCM (v/v) and lastly MeOH. The product co-eluted with triphenylphosphine oxide as the major impurity from 10% MeOH/DCM (v/v) to MeOH elution. After concentration of the product containing fractions under vacuum obtained Intermediate 2-B (2.10 g, 67%, 69% purity) as an orange oil.

### Step 3: tert-Butyl N-{1-[(4-nitrophenyl)formamido]-3,6,9-trioxa-12-azatetradecan-14-yl}carbamate (Intermediate 2-C)

tert-Butyl N-[2-(N-{2-[2-(2-{2-[(4-nitrophenyl)formamido]ethoxy}ethoxy)ethoxy]ethyl}2,4-dinitrobenzenesulfonamido)ethyl]carbamate (Intermediate 2-B, 2.10 g, 2.03 mmol, 69% purity) was dissolved in DCM (40 mL) and then n-propylamine (3.40 mL, 40.6 mmol) was slowly added at room temperature. The reaction was stirred at room temperature for 10 min and was found to have went to completion by HPLC-MS. The reaction was worked up by concentrating under vacuum and then purified by silica gel column chromatography. The crude sample was dry packed on silica gel and eluted with the following steps: EtOAc to 10% MeOH/90% DCM (v/v) to DCM/MeOH/7 M NH₃ in MeOH (70:10:1 ratio resp. to 50:10:1 ratio resp.) with the product eluted in the later part of the gradient. After concentration of the product containing fractions under vacuum obtained Intermediate 2-C (618 mg, 60%, 96% purity) as a pale orange oil.

### Step 4: tert-Butyl N-(2-{1-[1-(benzyloxy)-6-oxopyridin-2-yl]-N-{2-[2-(2-{2-[(4-nitrophenyl)formamido]ethoxy}ethoxy)ethoxy]ethyl}formamido}ethyl)carbamate (Intermediate 2-D)

To a solution of 1-(benzyloxy)-6-oxo-1,6-dihydropyridine-2-carboxylic acid chloride (J. Med. Chem. 2014, 57, 4849-4860) (196 mg, 0.74 mmol) dissolved in anhydrous MeCN (2 mL) was added DIPEA (261 μL, 1.49 mmol) and then a solution of tert-butyl N-{1-[(4-nitrophenyl)formamido]-3,6,9-trioxa-12-azatetradecan-14-yl}carbamate (Intermediate 2-C, 250 mg, 0.50 mmol as a 1.0 M solution in anhydrous MeCN) was added at room temperature. The reaction progress was monitored by HPLC-MS. The progression of the reaction had stalled at 80% conversion after 4 h so HBTU (192 mg, 0.50 mmol) was added and the reaction was stirred for an additional 1 h at room temperature which drove the reaction to completion. The reaction was worked up by concentration under vacuum and then purified by silica gel column chromatography by elution with 10% MeOH/DCM (v/v) to afford Intermediate 2-D (406 mg, 99%, 86% purity) as an orange oil.

### Step 5: Synthesis of N-{2-[2-(2-{2-[N-(2-Aminoethyl)-1-[1-(benzyloxy)-6-oxopyridin-2-yl]formamido]ethoxy}ethoxy)ethoxy]ethyl}-4-nitrobenzamide (Intermediate 2-E)

To a 20 mL scintillation vial containing tert-butyl N-(2-{1-[1-(benzyloxy)-6-oxopyridin-2-yl]-N-{2-[2-(2-{2-[(4-nitrophenyl)formamido]ethoxy}ethoxy) ethoxy]ethyl}formamido}ethyl)carbamate (Intermediate 2-D, 200 mg, 0.24 mmol) and a stir bar was added anhydrous DCM and then stirred at 0° C. in an ice bath. Next trifluoroacetic acid (370 μL, 4.83 mmol) was added and following the addition the reaction was stirred at room temperature and the reaction progress was monitored by HPLC-MS. Upon completion the reaction was worked up by concentrating under an airstream. The crude residue was then triturated with Et₂O (3×7 mL) to afford Intermediate 2-E (129 mg, 74%) as a pale orange oily residue as the TFA salt.

### Step 6: Synthesis of tert-Butyl-4-[(2-{-[1-(benzyloxy)-6-oxopyridin-2-yl]-N-{2-[2-(2-{2-[(4-nitrophenyl)formamido]ethoxy}ethoxy)ethoxy]ethyl}formamido}ethyl)carbamoyl]-2-{4,7,10-tris[2-(tert-butoxy)-2-oxoethyl]-1,4,7,10-tetraazacyclododecan-1-yl}butanoate (Intermediate 2-F)

To a solution of DOTAGA(tBu)₄ (70 mg, 0.10 mmol) in anhydrous MeCN (500 μL) was added HBTU (38 mg, 0.10 mmol) and stirred at room temperature for 5 min and then the TFA salt of N-{2-[2-(2-{2-[N-(2-aminoethyl)-1-[1-(benzyloxy)-6-oxopyridin-2-yl]formamido]ethoxy}ethoxy)ethoxy]ethyl}-4-nitrobenzamide (Intermediate 2-E, 64 mg, 89 μmol) dissolved in anhydrous MeCN (500 μL) with DIPEA (57.6 μL, 0.33 mmol) was added. The resulting solution was stirred at room temperature and the reaction progress was monitored by HPLC-MS. Upon completion the reaction was purified on a preparative C18 HPLC column to afford Intermediate 2-F (122 mg, 86%) as a clear film as the TFA salt.

### Step 7: Synthesis of 4-({2-[1-(1-Hydroxy-6-oxopyridin-2-yl)-N-{2-[2-(2-{2-[(4-nitrophenyl)formamido]ethoxy}ethoxy)ethoxy]ethyl}formamido]ethyl}carbamoyl)-2-[4,7,10-tris(carboxymethyl)-1,4,7,10-tetraazacyclododecan-1-yl]butanoic Acid (Intermediate 2-G)

To a 20 mL scintillation vial containing tert-butyl-4-[(2-{1-[1-(benzyloxy)-6-oxopyridin-2-yl]-N-{2-[2-(2-{2-[(4-nitrophenyl)formamido]ethoxy}ethoxy)ethoxy]ethyl}formamido}ethyl)carbamoyl]-2-{4,7,10-tris[2-(tert-butoxy)-2-oxoethyl]-1,4,7,10-tetraazacyclododecan-1-yl}butanoate (Intermediate 2-F, 97.5 mg, 56.4 μmol) and a stir bar was added AcOH (3 mL) followed by HCl (3 mL, 12 M, trace metals analysis grade). The resulting solution was capped and stirred in a 50° C. oil bath and the reaction progress was monitored by HPLC-MS. Upon completion the reaction was concentrated under an air stream and then purified on a preparative C18 HPLC column to afford Intermediate 2-G (29.7 mg, 43%) as a colourless film as the TFA salt.

### Step 8: Synthesis 4-{[2-(N-{2-[2-(2-{2-[(4-Aminophenyl)formamido]ethoxy}ethoxy)ethoxy]ethyl}-1-(1-hydroxy-6-oxopyridin-2-yl)formamido)ethyl]carbamoyl}-2-[4,7,10-tris(carboxymethyl)-1,4,7,10-tetraazacyclododecan-1-yl]butanoic Acid (Intermediate 2-H)

To a solution of 4-({2-[1-(1-Hydroxy-6-oxopyridin-2-yl)-N-{2-[2-(2-{2-[(4-nitrophenyl)formamido]ethoxy}ethoxy)ethoxy]ethyl}formamido]ethyl}carbamoyl)-2-[4,7,10 tris(carboxymethyl)-1,4,7,10-tetraazacyclododecan-1-yl]butanoic acid (Intermediate 2-G, 29.7 mg, 24.1 μmol) in methanol (3.6 mL) within a 20 mL scintillation vial with a stir bar was added Pd (10%)/C (26.0 mg, 24.4 μmol) and lastly ammonium formate (155 mg, 241 mmol). The reaction was then left to stir at room temperature and the reaction progress was monitored by HPLC-MS. Upon completion the reaction was diluted with methanol (3 mL), filtered through a 0.2 μm syringe filter, concentrated under vacuum and lastly purified on a preparative C18 HPLC column to afford Intermediate 2-H (12.6 mg, 44%) as a clear residue as the TFA salt.

### Step 9: Synthesis of 4-({2-[1-(1-Hydroxy-6-oxopyridin-2-yl)-N-{2-[2-(2-{2-[(4-isothiocyanatophenyl)formamido]ethoxy}ethoxy)ethoxy]ethyl}formamido]ethyl}carbamoyl)-2-[4,7,10-tris(carboxymethyl)-1,4,7,10-tetraazacyclododecan-1-yl]butanoic Acid (Compound B)

To a solution of 4-{[2-(N-{2-[2-(2-{2-[(4-Aminophenyl)formamido]ethoxy}ethoxy)ethoxy]ethyl}-1-(1-hydroxy-6-oxopyridin-2-yl)formamido)ethyl]carbamoyl}-2-[4,7,10-tris(carboxymethyl)-1,4,7,10-tetraazacyclododecan-1-yl]butanoic acid (Intermediate 2-H, 3.4 mg, 2.9 μmol) in 0.72 mL of 80% MeCN/20% H₂O (v/v) with a stir bar was added NEt₃ (1.12 μL, 8.0 μmol); the solution was then put in an ice bath and lastly di(2-pyridyl) thionocarbonate (1.2 mg, 5.0 μmol) was added. The solution was then allowed to stir at 0° C. and the reaction progress was monitored by HPLC-MS. Upon completion the reaction was worked up by purification on a preparative C18 HPLC column to afford Compound B (3.4 mg, 81%) as a white solid as the TFA salt following lyophilization. An aliquot was analyzed by HPLC-MS elution using elution method 2; retention time: 2.59 min; MS (positive ESI): found m/z 991.9 [M+H]⁺; C₄₃H₆₂N₉O₁₆S (calc. 992.4).

### Example 4: Synthesis of 4-[(2-{N-[2-(2-{2-[2-(3-{2-[2-(2-Azidoethoxy)ethoxy]ethoxy}propanamido)ethoxy]ethoxy}ethoxy)ethyl]-1-(1-hydroxy-6-oxopyridin-2-yl)formamido}ethyl)carbamoyl]-2-[4,7,10-tris(carboxymethyl)-1,4,7,10-tetraazacyclododecan-1-yl]butanoic Acid (Compound C)

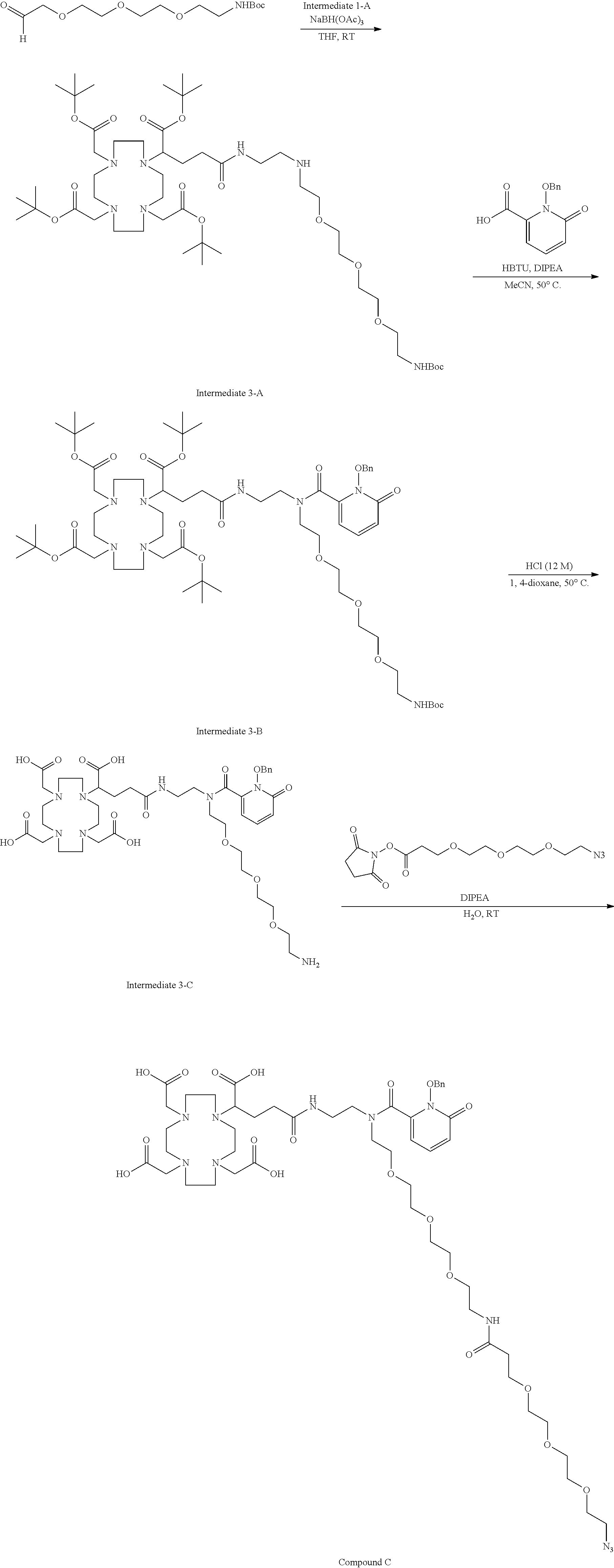

### Step 1: Synthesis of tert-Butyl-4-({1-[(tert-butoxycarbonyl)amino]-3,6,9-trioxa-12-azatetradecan-14-yl}carbamoyl)-2-{4,7,10-tris[2-(tert-butoxy)-2-oxoethyl]-1,4,7,10-tetraazacyclododecan-1-yl}butanoate (Intermediate 3-A)

To a 50 mL round bottom flask with a stir bar was added tert-butyl-4-[(2-aminoethyl)carbamoyl]-2-{4,7,10-tris[2-(tert-butoxy)-2-oxoethyl]-1,4,7,10-tetraazacyclododecan-1-yl}butanoate (Intermediate 1-A) TFA salt (253 mg, 0.26 mmol), tert-butyl N-(2-{2-[2-(2-oxoethoxy)ethoxy]ethoxy}ethyl)carbamate (101 mg, 0.31 mmol, ˜90% purity in 25 mL anhydrous THF) and lastly sodium triacetoxyborohydride (132 mg, 0.60 mmol) was added in one portion. The reaction was stirred at room temperature with a balloon outlet and was monitored by HPLC-MS. The reaction was worked up by the addition of NaHCO₃ (2 mL, saturated aqueous solution) and then concentrated under vacuum to afford a white solid. The crude was then dissolved in a mixture of DCM (25 mL) and H₂O (25 mL) transferred to a separatory funnel and the organic layer was extracted. The aqueous was extracted with an additional 25 mL of DCM and then the organic layers were combined, washed with brine and then dried over sodium sulfate, filtered and concentrated under vacuum. The crude was then purified on a preparative C18 HPLC column to afford tert-butyl-4-({1-[(tert-butoxycarbonyl)amino]-3,6,9-trioxa-12-azatetradecan-14-yl}carbamoyl)-2-{4,7,10-tris[2-(tert-butoxy)-2-oxoethyl]-1,4,7,10-tetraazacyclododecan-1-yl}butanoate (Intermediate 3-A) (67 mg, 21%) as a pale yellow residue as the TFA salt.

### Step 2: Synthesis of tert-Butyl-4-[(2-{-[1-(benzyloxy)-6-oxopyridin-2-yl]-N-{2-[2-(2-{2-[(tert-butoxycarbonyl)amino]ethoxy}ethoxy)ethoxy]ethyl}formamido}ethyl)carbamoyl]-2-{4,7,10-tris[2-(tert-butoxy)-2-oxoethyl]-1,4,7,10-tetraazacyclododecan-1-yl}butanoate (Intermediate 3-B)

To a solution of 1-(benzyloxy)-6-oxo-1,6-dihydropyridine-2-carboxylic acid (20.9 mg, 81 μmol) in anhydrous MeCN (2 mL) was added HBTU (31.7 mg, 81 μmol) and stirred at room temperature for 5 min and then tert-butyl-4-({1-[(tert-butoxycarbonyl)amino]-3,6,9-trioxa-12-azatetradecan-14-yl}carbamoyl)-2-{4,7,10-tris[2-(tert-butoxy)-2-oxoethyl]-1,4,7,10-tetraazacyclododecan-1-yl}butanoate (Intermediate 3-A, 67.3 mg, 54 μmol) dissolved in anhydrous MeCN (1 mL) with DIPEA (57 μL, 324 μmol) was added. The resulting solution was stirred in a 50° C. oil bath and the reaction was monitored by HPLC-MS. Upon completion the reaction was concentrated under vacuum and then purified on a preparative C18 HPLC column to afford Intermediate 3-B (48 mg, 48%, ˜80% purity) as a clear film as the TFA salt.

### Step 3: 4-({2-[N-(2-{2-[2-(2-Aminoethoxy)ethoxy]ethoxy}ethyl)-1-(1-hydroxy-6-oxopyridin-2-yl)formamido]ethyl}carbamoyl)-2-[4,7,10-tris(carboxymethyl)-1,4,7,10-tetraazacyclododecan-1-yl]butanoic Acid (Intermediate 3-C)

A vial containing tert-butyl-4-[(2-{1-[1-(benzyloxy)-6-oxopyridin-2-yl]-N-{2-[2-(2-{2-[(tert-butoxycarbonyl)amino]ethoxy}ethoxy)ethoxy]ethyl}formamido}ethyl) carbamoyl]-2-{4,7,10-tris[2-(tert-butoxy)-2-oxoethyl]-1,4,7,10-tetraazacyclododecan-1-yl}butanoate (Intermediate 3-B, 14.1 mg, 8.13 μmol, ˜85% purity) was charged with a stir bar, anhydrous 1,4-dioxane (1.5 mL), HCl (1.5 mL, 12 M, trace metals grade) and then the vial was capped. The resulting solution was stirred in a 50° C. oil bath and the reaction progress was monitored by HPLC-MS. Upon completion the reaction was concentrated under an air stream and then purified on a preparative C18 HPLC column to afford Intermediate 3-C (6.5 mg, 76%) as a clear film as the TFA salt.

### Step 4: Synthesis of 4-[(2-{N-[2-(2-{2-[2-(3-{2-[2-(2-Azidoethoxy)ethoxy]ethoxy}propanamido)ethoxy]ethoxy}ethoxy)ethyl]-1-(1-hydroxy-6-oxopyridin-2-yl)formamido}ethyl)carbamoyl]-2-[4,7,10-tris(carboxymethyl)-1,4,7,10-tetraazacyclododecan-1-yl]butanoic Acid (Compound C)

To a 20 mL vial with a stir bar was added 3.5 mg of 4-({2-[N-(2-{2-[2-(2-aminoethoxy)ethoxy]ethoxy}ethyl)-1-(1-hydroxy-6-oxopyridin-2-yl)formamido]ethyl}carbamoyl)-2-[4,7,10-tris(carboxymethyl)-1,4,7,10-tetraazacyclododecan-1-yl]butanoic acid (Intermediate 3-C, 3.5 mg, 3.3 μmol, c=2.0 mg/mL solution in Trace Select grade H₂O) followed by the addition of DIPEA (14.4 μL, 83 μmol). Lastly, Azido-PEG3-NHS (3.5 mg, 9.9 μmmol) was added as a freshly dissolved solution in H₂O (100 μL of Trace Select grade H₂O) and then reaction solution was stirred at room temperature. The reaction progress was monitored by HPLC-MS and upon completion the reaction was worked up by concentration under vacuum and then purified on a preparative C18 HPLC column to afford Compound C (3.2 mg, 75%) as a clear film as the TFA salt. An aliquot was analyzed by HPLC-MS elution using elution method 2; retention time: 1.80 min, 2.28 min and 2.52 min (75:9:16 ratio respectively) observing [M+H]⁺ and/or [M+Na]⁺; MS (positive ESI): found m/z 1060.1 [M+H]⁺; C₄₄H₇₄N₁₁O₁₉ (calc. 1060.5).

### Example 5: Synthesis of 4-(Propylcarbamoyl)-2-{4,7,10-tris[(1-hydroxy-6-oxopyridin-2-yl)methyl]-1,4,7,10-tetraazacyclododecan-1-yl}butanoic Acid (Compound D)

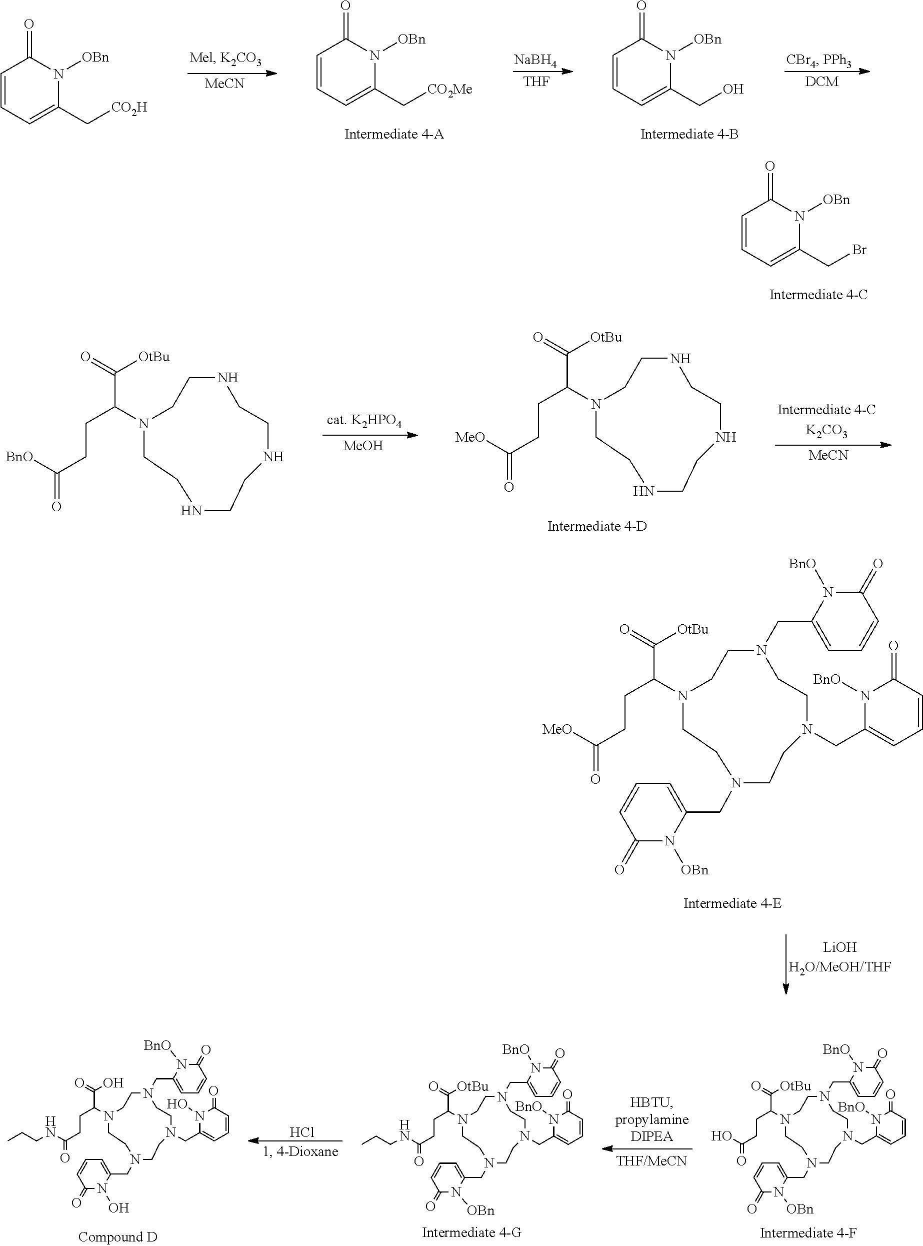

### Step 1: Synthesis of Methyl 1-(benzyloxy)-6-oxo-1,6-dihydropyridine-2-carboxylate (Intermediate 4-A)

A 20 mL scintillation vial was charged with 1-(benzyloxy)-6-oxo-1,6-dihydropyridine-2-carboxylic acid (200 mg, 815 μmol) followed by potassium carbonate (225 mg, 1.63 mmol) and 5 mL anhydrous acetonitrile and 5 mL anhydrous tetrahydrofuran. Iodomethane (110 uL, 1.77 mmol) was added and the vial was sealed and stirred at 40° C. for 16 h. An additional portion of iodomethane (55 uL 885 μmol) was then added and the reaction was continued for an additional 24 h. The solids were then removed by filtration and the filtrate was concentrated to dryness under reduced pressure. The residue was dissolved in 4 mL dichloromethane and residual solids were removed by a 2^{nd} filtration. The mother liquor was co-evaporated with 2×3 mL acetonitrile to afford methyl 1-(benzyloxy)-6-oxo-1,6-dihydropyridine-2-carboxylate (Intermediate 4-A) as a clear yellow oil (214 mg, 98% purity by HPLC, 99% yield).

### Step 2: Synthesis of 1-(Benzyloxy)-6-(hydroxymethyl)-1,2-dihydropyridin-2-one (Intermediate 4-B)

A 25 mL round bottom flask was charged with 1-(benzyloxy)-6-oxo-1,6-dihydropyridine-2-carboxylic acid methyl ester (Intermediate 4-A, 214 mg, 829 μmol) followed by NaBH₄ (385 mg, 9.95 mmol) and 8 mL anhydrous tetrahydrofuran. The flask was then affixed with a reflux condenser and a nitrogen balloon and heated to reflux for 16 h. The reaction mass was then cooled to 0-5° C. and quenched with the slow addition of 5 mL of methanol. The mixture was concentrated to dryness under reduced pressure and then dissolved in a mixture of dichloromethane and water. 2 mL of saturated ammonium chloride solution was added, and the phases were separated by separatory funnel. The aqueous phase was extracted with 4×20 mL dichloromethane, the organics were combined and dried over Na₂SO₄ (s). Solids were removed by filtration, washed with 3×20 mL dichloromethane and the filtrate was concentrated under reduced pressure to afford 1-(benzyloxy)-6-(hydroxymethyl)-1,2-dihydropyridin-2-one (Intermediate 4-B) as a waxy white solid (144 mg, 85% purity by HPLC, 64% yield).

### Step 3: Synthesis of 1-(Benzyloxy)-6-(bromomethyl)pyridine-2-one (Intermediate 4-C)

A 20 mL scintillation vial was charged with 1-(benzyloxy)-6-(hydroxymethyl)-1,2-dihydropyridin-2-one (Intermediate 4-B, 63 mg, 272 μmol) followed by tetrabromomethane (135 mg, 409 μmol) and 2 mL of anhydrous dichloromethane. The mixture was then cooled in an ice-water bath. After 10 minutes of cooling, triphenylphosphine (110 mg, 409 μmol) was added portion wise as a solid over 10 mins. After another 10 minutes the reaction was checked by TLC and confirmed to be complete. The reaction was quenched with 0.5 mL saturated sodium sulfite (Na₂SO₃) solution and allowed to stir at room temperature for 30 mins. The reaction was then transferred to a separatory funnel, extracted into dichloromethane and the organics were dried over Na₂SO₄ (s). Solids were removed by filtration and the mother liquor was concentrated under reduced pressure to a residue. Purification by flash column chromatography on silica (eluent: 30% toluene in ethyl acetate) afforded 1-(benzyloxy)-6-(bromomethyl)pyridine-2-one (Intermediate 4-C) as a clear viscous oil that solidified to a white film on standing (63 mg, 75%).

### Step 4: Synthesis of 1-tert-Butyl 5-methyl-2-(1,4,7,10-tetraazacyclododecan-1-yl)pentanedioate (Intermediate 4-D)

To a 20 mL scintillation vial containing 5-benzyl 1-tert-butyl-2-(1,4,7,10-tetraazacyclododecan-1-yl)pentanedioate (Org. Process Res. Dev. 2009, 13, 535-542) (112 mg, 250 μmol) was charged potassium phosphate dibasic (4.5 mg, 25 μmol, 0.1 equiv.) and 4 mL of methanol and the reaction vial was heated to 75° C. for 3.5 h. An additional portion of potassium phosphate dibasic was then added (10 mg, 57 μmol, 0.2 equiv.) and the reaction was maintained at 75° C. for an additional 16 h. The mixture was then cooled to room temperature and concentrated to dryness under reduced pressure. The resulting residue was dissolved in 1 mL of 1:1 water:acetonitrile, filtered through a 0.2 μm filter and then purified by preparative C18 HPLC. 1-Tert-butyl 5-methyl-2-(1,4,7,10-tetraazacyclododecan-1-yl)pentanedioate (Intermediate 4-D) was obtained as a pale-yellow oil (61 mg, 41% yield as the TFA salt).

### Step 5: Synthesis of 1-tert-Butyl 5-methyl 2-[4,7,10-tris({[1-(benzyloxy)-6-oxopyridin-2-yl]methyl})-1,4,7,10-tetraazacyclododecan-1-yl]pentanedioate (Intermediate 4-E)

A 20 mL scintillation vial was charged with 1-(benzyloxy)-6-(bromomethyl)-1,2-dihydropyridin-2-one (Intermediate 4-C, 33 mg, 165 μmol) 1-tert-butyl 5-methyl 2-(1,4,7,10-tetraazacyclododecan-1-yl)pentanedioate (Intermediate 4-D, 20 mg, 53.7 μmol) and potassium carbonate (46.8 mg, 165 μmol) followed by 2 mL anhydrous acetonitrile. The vial headspace was purged with nitrogen, the vial then capped and heated in an oil bath at 50° C. for 4 hours and 20 minutes. The mixture was then cooled to room temperature and concentrated to a residue. The residue was triturated in 4 mL of dichloromethane and then filtered to remove the insoluble solids. The filtrate was concentrated to dryness under reduced pressure and the resulting residue was dissolved in 2 mL of a 1:1 acetonitrile:water mixture. This solution was filtered through a 0.2 μm filter and then purified by preparative C18 HPLC to afford 1-tert-butyl 5-methyl 2-[4,7,10-tris({[1-(benzyloxy)-6-oxopyridin-2-yl]methyl})-1,4,7,10-tetraazacyclododecan-1-yl]pentanedioate (Intermediate 4-E) as small colourless particles in (28 mg, 68% purity as determined by HPLC, 29% yield as the TFA salt). Intermediate 4-E was carried forward without additional purification.

### Step 6: Synthesis of 5-(tert-Butoxy)-5-oxo-4-[4,7,10-tris({[1-(benzyloxy)-6-oxopyridin-2-yl]methyl})-1,4,7,10-tetraazacyclododecan-1-yl]pentanoic Acid (Intermediate 4-F)

A 20 mL scintillation vial was charged with 1-tert-butyl 5-methyl 2-[4,7,10-tris({[1-(benzyloxy)-6-oxo-1,6-dihydropyridin-2-yl]methyl})-1,4,7,10-tetraazacyclododecan-1-yl]pentanedioate (Intermediate 4-E, 28 mg, 18.8 μmol, 68% purity as determined by HPLC) followed by lithium hydroxide (1.5 mg, 230 μmol) then 1.5 mL of a 1:1:1 mixture of water:tetrahydrofuran:methanol and the solution was stirred at ambient temperature. After 1.5 h an additional portion of lithium hydroxide was added (4 mg, 167 μmol) and the reaction was maintained at room temperature for an additional 5 h. The reaction mixture was then concentrated to a residue under reduced pressure and then dissolved in 2 mL of a 1:1 mixture of acetonitrile:0.1% trifluoroacetic acid in water. This solution was passed through a 0.2 μm filter and then purified by preparative C18 HPLC to afford 5-(tert-butoxy)-5-oxo-4-[4,7,10-tris({[1-(benzyloxy)-6-oxopyridin-2-yl]methyl})-1,4,7,10-tetraazacyclododecan-1-yl]pentanoic acid (Intermediate 4-F) as a clear and colourless film (17 mg, 91% pure as determined by HPLC, 67% yield as the TFA salt). Intermediate 4-F was carried forward in subsequent steps without further purification.

### Step 7: Synthesis of tert-Butyl 4-(propylcarbamoyl)-2-[4,7,10-tris({[1-(benzyloxy)-6-oxo-1,6-dihydropyridin-2-yl]methyl})-1,4,7,10-tetraazacyclododecan-1-yl]butanoate (Intermediate 4-G)

To a 20 mL scintillation vial containing 5-(tert-butoxy)-5-oxo-4-[4,7,10-tris({[1-(benzyloxy)-6-oxo-1,6-dihydropyridin-2-yl]methyl})-1,4,7,10-tetraazacyclododecan-1-yl]pentanoic acid (Intermediate 4-F, 17 mg, 15.5 μmol) was added HBTU (7.1 mg, 18.6 μmol) and then 1 mL anhydrous acetonitrile and 1 mL anhydrous tetrahydrofuran. Diisopropylethylamine (13.5 uL, 77.5 μmol) was then added and the mixture was stirred at ambient temperature for 25 minutes. Propylamine (2.55 uL, 31 μmol) was then added and the mixture was maintained at ambient temperature for an additional 1 h 15 minutes. The reaction was then concentrated under reduced pressure to a residue, dissolved in 2 mL 1:1 acetonitrile:water, filtered through a 0.2 μm filter and purified by preparative C18 HPLC. Tert-butyl 4-(propylcarbamoyl)-2-[4,7,10-tris({[1-(benzyloxy)-6-oxo-1,6-dihydropyridin-2-yl]methyl})-1,4,7,10-tetraazacyclododecan-1-yl]butanoate (Intermediate 4-G) was obtained as a clear film (14.5 mg, 94% purity by HPLC, 70% yield as the TFA salt). Intermediate 4-G was used in subsequent steps without further purification.

### Step 8: Synthesis of 4-(Propylcarbamoyl)-2-{4,7,10-tris[(1-hydroxy-6-oxopyridin-2-yl)methyl]-1,4,7,10-tetraazacyclododecan-1-yl}butanoic Acid (Compound D)

To a 20 mL scintillation vial containing tert-butyl 4-(propylcarbamoyl)-2-[4,7,10-tris({[1-(benzyloxy)-6-oxo-1,6-dihydropyridin-2-yl]methyl})-1,4,7,10-tetraazacyclododecan-1-yl]butanoate (Intermediate 4-G, 14.5 mg, 13.95 μmol) and a stir bar was added anhydrous 1,4-dioxane (0.5 mL) and HCl (12 M, 0.5 mL). The resulting solution was capped and stirred in an oil bath at 50° C. for 4 h. The mixture was then cooled to room temperature and concentrated under an air stream to a thin residue. 4 mL of acetonitrile was added, and the mixture was concentrated under reduced pressure to a residue. This was repeated an additional three times with 3 mL acetonitrile for each repetition. The resulting residue was dissolved in 1 mL 0.1% trifluoroacetic acid in water and purified by preparative C18 HPLC to afford 4-(propylcarbamoyl)-2-{4,7,10-tris[(1-hydroxy-6-oxopyridin-2-yl)methyl]-1,4,7,10-tetraazacyclododecan-1-yl}butanoic acid (Compound D) as a clear colourless film (5.0 mg, 30% yield as the TFA salt, >80% purity as determined by HPLC). An aliquot was analyzed by HPLC elution method 3; retention time=3.6 mins; MS (positive ESI): found m/z=713.0 [M+H]⁺; C₃₄H₄₉N₈O₉ (calc. 713.4).

### Example 6: Synthesis of [7-(Carboxymethyl)-4,10-bis[(1-hydroxy-6-oxopyridin-2-yl)methyl]-1,4,7,10-tetraazacyclododecan-1-yl]acetic Acid (Compound E)

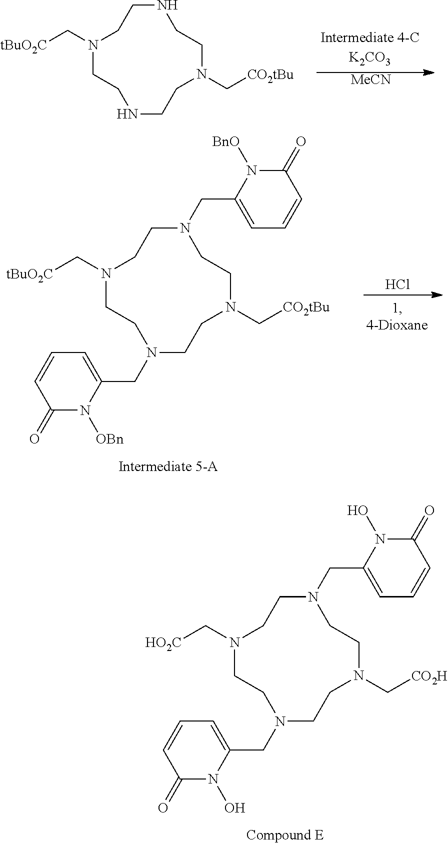 [SMILES: "MeCN" ; CC(C)(C)OC(=O)CN1CCNCCN(CC(=O)OC(C)(C)C)CCNCC1 ; CC(C)(C)OC(=O)CN1CCN(Cc2cccc(=O)n2OCc2ccccc2)CCN(CC(=O)OC(C)(C)C)CCN(Cc2cccc(=O)n2OCc2ccccc2)CC1 ; Cl ; O=C(O)CN1CCN(Cc2cccc(=O)n2O)CCN(CC(=O)O)CCN(Cc2cccc(=O)n2O)CC1]

### Step 1: Synthesis of tert-Butyl 2-[4,10-bis({[1-(benzyloxy)-6-oxopyridin-2-yl]methyl})-7-[2-(tert-butoxy)-2-oxoethyl]-1,4,7,10-tetraazacyclododecan-1-yl]acetate (Intermediate 5-A)

A 20 mL scintillation vial was charged with 1-(benzyloxy)-6-(bromomethyl)-1,2-dihydropyridin-2-one (Intermediate 4-C, 12.2 mg, 41.5 μmol) tert-butyl 2-{7-[2-(tert-butoxy)-2-oxoethyl]-1,4,7,10-tetraazacyclododecan-1-yl}acetate (Dalton Trans. 2016, 45, 4791-4801) (8 mg, 20 μmol) and potassium carbonate (13 mg, 41.5 μmol) followed by 2 mL anhydrous acetonitrile. The vial headspace was purged with nitrogen and then sealed and heated in an oil bath at 50° C. for 3.5 h. The insoluble solids were then removed by filtration and the mother liquor was concentrated under reduced pressure. The residue was dissolved in 1 mL 1:1 acetonitrile:water and filtered through a 0.2 μm filter. The residue was purified by preparative C18 HPLC to afford tert-butyl 2-[4,10-bis({[1-(benzyloxy)-6-oxopyridin-2-yl]methyl})-7-[2-(tert-butoxy)-2-oxoethyl]-1,4,7,10-tetraazacyclododecan-1-yl]acetate (Intermediate 5-A) as small colourless particles, pertaining to the product as a mixture, which was carried forward in subsequent steps without further purification (20.5 mg, 68% purity as determined by HPLC, 66% yield as the TFA salt).

### Step 2: Synthesis of [7-(Carboxymethyl)-4,10-bis[(1-hydroxy-6-oxopyridin-2-yl)methyl]-1,4,7,10-tetraazacyclododecan-1-yl]acetic Acid (Compound E)

To a 20 mL scintillation vial containing tert-butyl 2-[4,10-bis({[1-(benzyloxy)-6-oxopyridin-2-yl]methyl})-7-[2-(tert-butoxy)-2-oxoethyl]-1,4,7,10-tetraazacyclododecan-1-yl]acetate (Intermediate 5-A, 19.4 μmol) and a stir bar, was added anhydrous 1,4-dioxane (1 mL) and HCl (12 M, 1 mL). The resulting solution was capped and stirred in an oil bath at 50° C. for 7 h. The reaction mixture was then concentrated under a stream of compressed air and then co-evaporated with 2 mL water under reduced pressure to provide a clear and colourless residue. The residue was dissolved in 1 mL 0.1% trifluoroacetic acid in water, the solution was passed through a 0.2 μm filter and then purified by preparative C18 HPLC to afford [7-(Carboxymethyl)-4,10-bis[(1-hydroxy-6-oxopyridin-2-yl)methyl]-1,4,7,10-tetraazacyclododecan-1-yl]acetic acid (Compound E) as a clear colourless film (7.2 mg, 93% purity as determined by HPLC, 46% yield as the TFA salt). An aliquot was analyzed by HPLC elution method 3; retention time=1.2 mins; MS (positive ESI): found m/z=534.8 [M+H]⁺; C₂₄H₃₅N₆O₈ (calc. 535.3).

### Example 7: Synthesis of 1-Hydroxy-6-({4,7,10-tris[(1-hydroxy-6-oxopyridin-2-yl)methyl]-1,4,7,10-tetraazacyclododecan-1-yl}methyl)pyridin-2-one (Compound F)

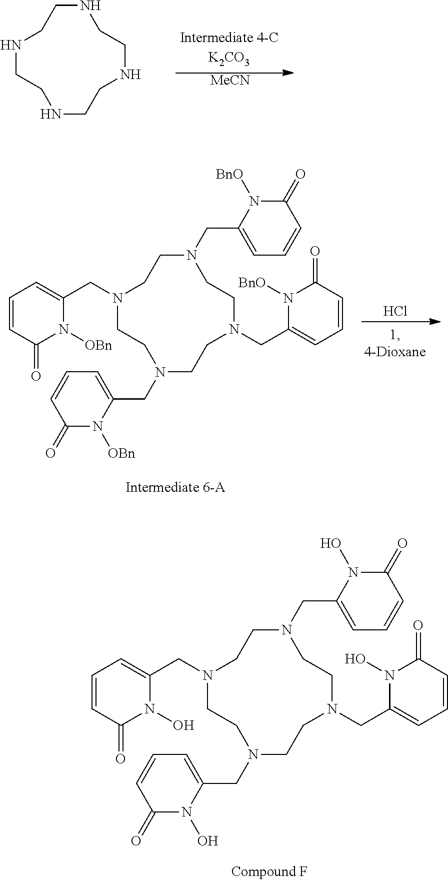 [SMILES: "MeCN" ; C1CNCCNCCNCCN1 ; O=c1cccc(CN2CCN(Cc3cccc(=O)n3OCc3ccccc3)CCN(Cc3cccc(=O)n3OCc3ccccc3)CCN(Cc3cccc(=O)n3OCc3ccccc3)CC2)n1OCc1ccccc1 ; Cl ; O=c1cccc(CN2CCN(Cc3cccc(=O)n3O)CCN(Cc3cccc(=O)n3O)CCN(Cc3cccc(=O)n3O)CC2)n1O]

### Step 1: Synthesis of 1-(Benzyloxy)-6-{[4,7,10-tris({[1-(benzyloxy)-6-oxopyridin-2-yl]methyl})-1,4,7,10-tetraazacyclododecan-1-yl]methyl}pyridin-2-one (Intermediate 6-A)

A 20 mL scintillation vial was charged with Intermediate 4-C (33 mg, 107 μmol), cyclen (4.7 mg, 27.3 μmol) and potassium carbonate (31 mg, 224 μmol) followed by 2 mL of anhydrous acetonitrile. The vial headspace was purged with nitrogen and then sealed and heated in an oil bath at 50° C. for 14 h. The reaction mass was then cooled to room temperature then concentrated to dryness under reduced pressure. The residue was dissolved in 1 mL of 1:1 acetonitirile:water mixture and then purified by preparative C18 HPLC to afford Intermediate 6-A as a light yellow viscous film (9.3 mg, 98% purity as determined by HPLC, 27% yield as the TFA salt).

### Step 2: 1-Hydroxy-6-({4,7,10-tris[(1-hydroxy-6-oxopyridin-2-yl)methyl]-1,4,7,10-tetraazacyclododecan-1-yl}methyl)pyridin-2-one (Compound F)

To a 20 mL scintillation vial containing Intermediate 6-A and a stir bar was added 0.5 mL of anhydrous 1,4-dioxane and 0.5 mL of 12 M hydrochloric acid. The reaction vial was capped and stirred at 50° C. for 1 h and 40 min. The reaction mixture was then cooled to room temperature and concentrated under an air stream. The residue was further co-evaporated with 4 mL of acetonitrile under reduced pressure. The resulting concentrate was dissolved in 1 mL 0.1% trifluoroacetic acid in water and then purified by preparative C18 HPLC to afford Compound F as an opaque colourless film (4.0 mg, 85% purity as determined by HPLC, 42% yield as the TFA salt). An aliquot was analyzed by HPLC elution method 3; retention time=3.9 mins; MS (positive ESI): found m/z=665.9 [M+H]⁺; C₃₂H₄₁N₈O₈ (calc. 665.3).

### Example 8: Synthesis of [4,7-Bis(carboxymethyl)-10-[(1-hydroxy-6-oxopyridin-2-yl)methyl]-1,4,7,10-tetraazacyclododecan-1-yl]acetic Acid (Compound G)

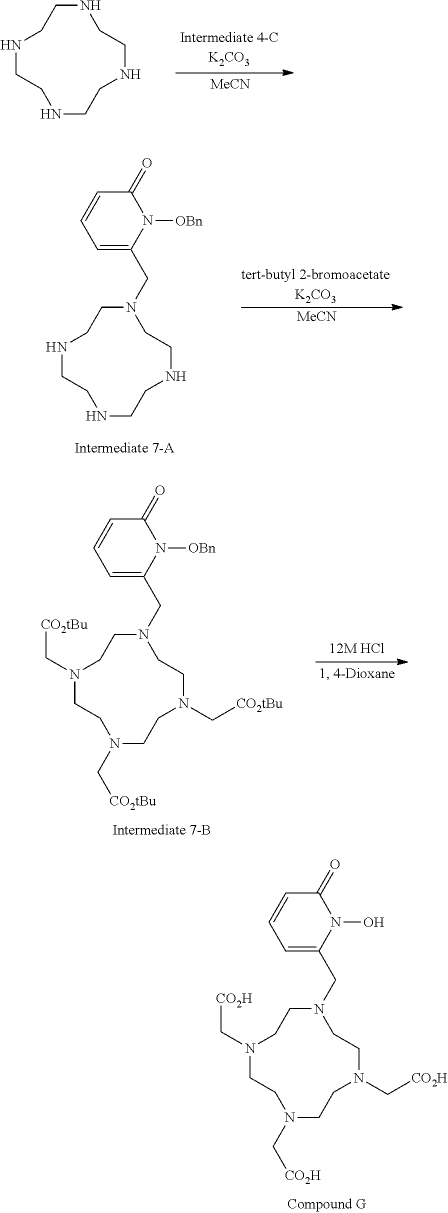

### Step 1: 1-(Benzyloxy)-6-(1,4,7,10-tetraazacyclododecan-1-ylmethyl)pyridin-2-one (Intermediate 7-A)

A 20 mL scintillation vial was charged with Intermediate 4-C (17 mg, 58 μmol), cyclen (20 mg, 117 μmol) and potassium carbonate (35 mg, 255 μmol) followed by 3 mL of anhydrous acetonitrile. The vial headspace was purged with nitrogen and then sealed and heated in an oil bath at 50° C. for 18 h. The reaction mass was then cooled to room temperature then concentrated to dryness under reduced pressure. The residue was triturated in dichloromethane (2×2 mL) and the solids were removed by filtration and the mother liquor concentrated to a residue. The mixture was dissolved in 1.5 mL of 2:1 of 0.1% trifluoroacetic acid in water:acetonitrile mixture and then purified by preparative C18 HPLC to afford Intermediate 7-A as a clear colourless film (28 mg, >98% purity as determined by HPLC, 79% yield as the TFA salt).

### Step 2: Tert-Butyl 2-(4-{[1-(benzyloxy)-6-oxopyridin-2-yl]methyl}-7,10-bis[2-(tert-butoxy)-2-oxoethyl]-1,4,7,10-tetraazacyclododecan-1-yl)acetate (Intermediate 7-B)

A 20 mL scintillation vial was charged with Intermediate 7-A (28 mg, 46 μmol), tert-butyl 2-bromoacetate (29.5 mg, 151 μmol) and potassium carbonate (39 mg, 284 μmol) followed by 3 mL of anhydrous acetonitrile. The vial headspace was purged with nitrogen and then sealed and heated in an oil bath at 50° C. for 14.5 h. The reaction mass was then cooled to room temperature then concentrated to dryness under reduced pressure. The residue was triturated in dichloromethane (2×2 mL) and the solids were removed by filtration and the mother liquor concentrated to a residue. The mixture was dissolved in 2 mL of 2:1 of acetonitrile:water mixture and then purified by preparative C18 HPLC to afford Intermediate 7-B as a clear colourless film (23 mg, >98% purity as determined by HPLC, 51% yield as the TFA salt).

### Step 3: [4,7-Bis(carboxymethyl)-10-[(1-hydroxy-6-oxopyridin-2-yl)methyl]-1,4,7,10-tetraazacyclododecan-1-yl]acetic Acid (Compound G)

A 20 mL scintillation vial was charged with Intermediate 7-B (23 mg, 32 μmol), 0.5 mL of anhydrous 1,4-dioxane and then 0.5 mL of 12 M hydrochloric acid. The vial headspace was purged with nitrogen and then sealed and heated in an oil bath at 50° C. for 18 h. The reaction mass was then cooled to room temperature then concentrated to dryness under a stream of compressed air and then co-evaporated with 4 mL Trace Select grade water under reduced pressure to provide a clear and colourless residue. The residue was dissolved in 1 mL of Trace Select grade water and then purified by preparative C18 HPLC to afford Compound G as a clear colourless film (8.8 mg, >93% purity as determined by HPLC, 37% yield as the TFA salt). An aliquot was analyzed by HPLC elution method 1; retention time=0.74 mins; MS (positive ESI): found m/z=469.8 [M+H]⁺; C₂₀H₃₂N₅O₈ (calc. 470.2).

### Example 9: Synthesis of {4,10-Bis[(1-hydroxy-6-oxopyridin-2-yl)methyl]-7-(phosphonomethyl)-1,4,7,10-tetraazacyclododecan-1-yl}methylphosphonic Acid (Compound H)

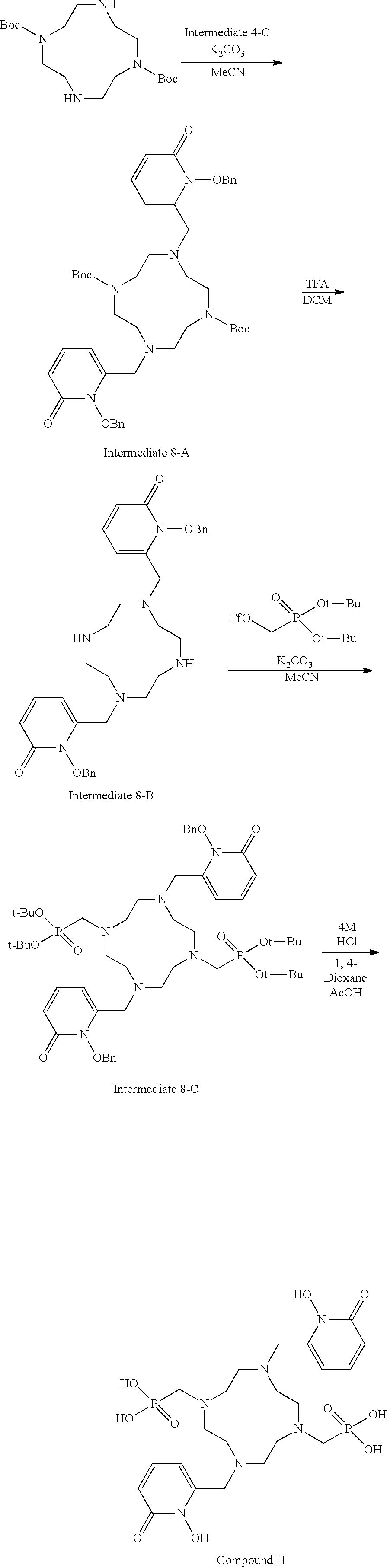

### Step 1: 1,7-Di-tert-butyl 4,10-bis({[1-(benzyloxy)-6-oxopyridin-2-yl]methyl})-1,4,7,10-tetraazacyclododecane-1,7-dicarboxylate (Intermediate 8-A)

A 20 mL scintillation vial was charged with 1,7-di-tert-butyl 1,4,7,10-tetraazacyclododecane-1,7-dicarboxylate (250 mg, 604 μmol), Intermediate 4-C (332 mg, 1.13 mmol) and potassium carbonate (297 mg, 2.15 mmol) followed by 3 mL of anhydrous acetonitrile and 0.5 g of molecular sieves. The vial headspace was purged with nitrogen and then sealed and heated in an oil bath at 50° C. for 19 h. The reaction mass was then cooled to room temperature, the solids were removed by filtration and the mother liquor was then concentrated to dryness under reduced pressure. The mixture was dissolved in 3 mL of 2:8 of water:acetonitrile mixture and then purified by preparative C18 HPLC to afford Intermediate 8-A as a clear light yellow heavy oil (646 mg, >85% purity as determined by HPLC, 91% yield as the TFA salt).

### Step 2: 1-(Benzyloxy)-6-[(7-{[1-(benzyloxy)-6-oxo-1,6-dihydropyridin-2-yl]methyl}-1,4,7,10-tetraazacyclododecan-1-yl)methyl]-1,2-dihydropyridin-2-one (Intermediate 8-B)

A 20 mL scintillation vial was charged with Intermediate 8-A (646 mg, 630 μmol) followed by 3 mL dichloromethane and then 1 mL trifluoroacetic acid. The reaction vessel was capped and maintained with stirring at 20-25° C. for 6.5 h. The reaction was then concentrated under a stream of compressed air then co-evaporated with 2×4 mL acetonitrile under reduced pressure to provide a clear and colourless viscous residue. The residue was dissolved in 5 mL of 3:1 of 0.1% trifluoroacetic acid in water:acetonitrile mixture and then purified by preparative C18 HPLC to afford Intermediate 8-B as a clear, light yellow heavy oil (362 mg, >98% purity as determined by HPLC, 70% yield as the TFA salt).

### Step 3: Di-tert-butyl [4,10-bis({[1-(benzyloxy)-6-oxopyridin-2-yl]methyl})-7-{[bis(tert-butoxy)phosphoryl]methyl}-1,4,7,10-tetraazacyclododecan-1-yl]methylphosphonate (Intermediate 8-C)

A 20 mL scintillation vial was charged with Intermediate 8-B (50 mg, 60.5 μmol) followed by [bis(tert-butoxy)phosphoryl]methyl trifluoromethanesulfonate (40 mg, 133 μmol) and potassium carbonate (26 mg, 181 μmol) then 2 mL of anhydrous acetonitrile. The vial headspace was purged with nitrogen and then sealed and heated in an oil bath at 50° C. for 18 h. An additional aliquot of [bis(tert-butoxy)phosphoryl]methyl trifluoromethanesulfonate (15 mg, 50 μmol) was added and the reaction was maintained at 50° C. for an additional 72 h. The reaction mass was then cooled to room temperature, the solids were removed by filtration and the mother liquor was concentrated to dryness under reduced pressure. The resulting mixture was dissolved in 1 mL of acetonitrile and then purified by preparative C18 HPLC to afford Intermediate 8-C as a mixture with the mono and di-phosphonic acid hydrolysis by-products in a ratio of 38:36:22 respectively. Isolated 21 mg of a clear colourless film (21 mg, mixture as described above, 25% yield as the TFA salt). Since all components were productive towards the desired product, the mixture was carried forward without further purification.

### Step 4: {4,10-Bis[(1-hydroxy-6-oxopyridin-2-yl)methyl]-7-(phosphonomethyl)-1,4,7,10-tetraazacyclododecan-1-yl}methylphosphonic Acid (Compound H)

A 20 mL scintillation vial was charged with a mixture of Intermediate 8-C (21 mg, approx. 15.3 μmol) followed by 1.5 mL each of 4M HCl in 1,4 dioxane and 4M HCl in acetic acid. The vial was then sealed and heated in an oil bath at 50° C. for 19 h. The reaction mass was then cooled to room temperature then concentrated to dryness under a stream of compressed air then co-evaporated with 3 mL Trace Select grade water under reduced pressure to provide a clear and colourless residue. The residue was dissolved in 1 mL of 0.1% trifluoroacetic acid in Trace Select grade water and then purified by preparative C18 HPLC to afford Compound H as an opaque light yellow chalky powder (11.6 mg, >98% purity as determined by HPLC, 91% yield as the TFA salt). An aliquot was analyzed by HPLC elution method 1; retention time=0.70 mins; MS (positive ESI): found m/z=607.0 [M+H]⁺; C₂₂H₃₇N₆O₁₀P₂ (calc. 607.2).

### Example 10: Synthesis of 1-Hydroxy-6-({4,8,11-tris[(1-hydroxy-6-oxopyridin-2-yl)methyl]-1,4,8,11-tetraazacyclotetradecan-1-yl}methyl)pyridin-2-one (Compound I)

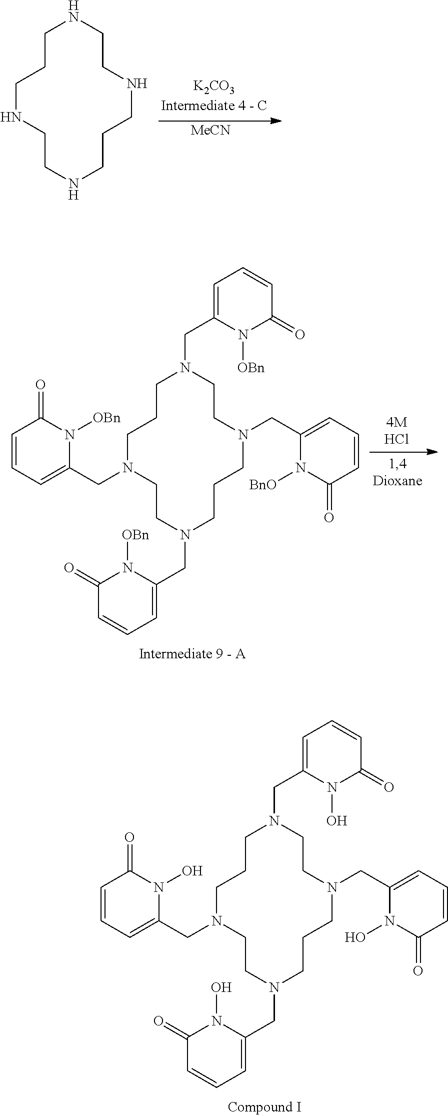 [SMILES: "MeCN" ; C1CNCCNCCCNCCNC1 ; O=c1cccc(CN2CCCN(Cc3cccc(=O)n3OCc3ccccc3)CCN(Cc3cccc(=O)n3OCc3ccccc3)CCCN(Cc3cccc(=O)n3OCc3ccccc3)CC2)n1OCc1ccccc1 ; O=c1cccc(CN2CCCN(Cc3cccc(=O)n3O)CCN(Cc3cccc(=O)n3O)CCCN(Cc3cccc(=O)n3O)CC2)n1O]

### Step 1: 1-(Benzyloxy)-6-{[4,8,11-tris({[1-(benzyloxy)-6-oxopyridin-2-yl]methyl})-1,4,8,11-tetraazacyclotetradecan-1-yl]methyl}pyridin-2-one (Intermediate 9-A)

A 20 mL scintillation vial was charged with 1,4,8,11-tetraazacyclotetradecane (cyclam, 30 mg, 135 μmol), Intermediate 4-C (198 mg, 674 μmol) and potassium carbonate (112 mg, 809 mmol) followed by 2 mL of anhydrous acetonitrile and 0.3 g of molecular sieves. The vial headspace was purged with nitrogen and then sealed and heated in an oil bath at 50° C. for 22.5 h. The reaction mass was then cooled to room temperature, the solids were removed by filtration and the mother liquor was then concentrated to dryness under reduced pressure. The mixture was dissolved in 2 mL of 2:3 of water:acetonitrile mixture and then purified by preparative C18 HPLC to afford Intermediate 9-A as a clear colourless viscous film (110 mg, >98% purity as determined by HPLC, 64% yield as the TFA salt).

### Step 2: 1-Hydroxy-6-({4,8,11-tris[(1-hydroxy-6-oxopyridin-2-yl)methyl]-1,4,8,11-tetraazacyclotetradecan-1-yl}methyl)pyridin-2-one (Compound I)

A 20 mL scintillation vial was charged with Intermediate 9-A (20 mg, 15.6 μmol) followed by 1 mL of 4 M hydrochloric acid in 1,4-dioxane. The reaction vessel was capped and maintained with stirring at 50° C. for 2 h. The reaction was then concentrated under a stream of compressed air then co-evaporated with 2×4 mL Trace Select grade water under reduced pressure to provide a clear and colourless film. The residue was dissolved in 1 mL of 7:3 of 0.1% trifluoroacetic acid in water:acetonitrile mixture and then purified by preparative C18 HPLC to afford Compound I as clear colourless film (6 mg, 98% purity as determined by HPLC, 42% yield as the TFA salt). An aliquot was analyzed by HPLC elution method 2; retention time=2.3 mins; MS (positive ESI): found m/z=692.9 [M+H]⁺; C₃₄H₄₅N₈O₈ (calc. 693.3).

### Example 11: Synthesis of 1-Hydroxy-6-({4,7,10,13,16-pentakis[(1-hydroxy-6-oxopyridin-2-yl)methyl]-1,4,7,10,13,16-hexaazacyclooctadecan-1-yl}methyl)pyridin-2-one (Compound J)

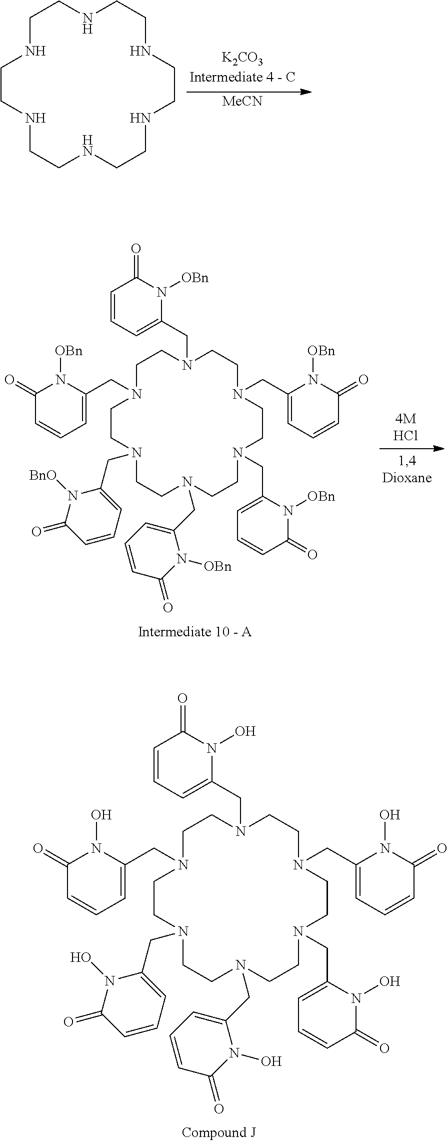 [SMILES: "MeCN" ; C1CNCCNCCNCCNCCNCCN1 ; O=c1cccc(CN2CCN(Cc3cccc(=O)n3OCc3ccccc3)CCN(Cc3cccc(=O)n3OCc3ccccc3)CCN(Cc3cccc(=O)n3OCc3ccccc3)CCN(Cc3cccc(=O)n3OCc3ccccc3)CCN(Cc3cccc(=O)n3OCc3ccccc3)CC2)n1OCc1ccccc1 ; O=c1cccc(CN2CCN(Cc3cccc(=O)n3O)CCN(Cc3cccc(=O)n3O)CCN(Cc3cccc(=O)n3O)CCN(Cc3cccc(=O)n3O)CCN(Cc3cccc(=O)n3O)CC2)n1O]

### Step 1: 1-(Benzyloxy)-6-{[4,7,10,13,16-pentakis({[1-(benzyloxy)-6-oxo-1,6-dihydropyridin-2-yl]methyl})-1,4,7,10,13,16-hexaazacyclooctadecan-1-yl]methyl}-1,2-dihydropyridin-2-one (Intermediate 10-A)

A 20 mL scintillation vial was charged with 1,4,8,11-tetraazacyclotetradecane trisulfate (hexacyclen trisulfate, 44 mg, 71.7 μmol), Intermediate 4-C (147 mg, 502 μmol) and potassium carbonate (119 mg, 860 μmol) followed by 2 mL of anhydrous acetonitrile and 0.4 g of molecular sieves. The vial headspace was purged with nitrogen and then sealed and heated in an oil bath at 50° C. for 19 h. At this point potassium tert-butoxide (24 mg, 214 μmol) was added as well as an additional 2 mL of anhydrous acetonitrile and the reaction was reheated to 50° C. for an additional 76 h. The reaction mass was then cooled to room temperature, the solids were removed by filtration and the mother liquor was concentrated to dryness under reduced pressure. The mixture was dissolved in 1.5 mL of 2:8 of water:acetonitrile mixture and then purified by preparative C18 HPLC to afford Intermediate 10-A as a thick yellow film (47 mg, 77% purity as determined by HPLC, 30% yield as the TFA salt).

### Step 2:1-Hydroxy-6-({4,7,10,13,16-pentakis[(1-hydroxy-6-oxopyridin-2-yl)methyl]-1,4,7,10,13,16-hexaazacyclooctadecan-1-yl}methyl)pyridin-2-one (Compound J)

A 20 mL scintillation vial was charged with Intermediate 10-A (47 mg, 77% purity as determined by HPLC, 21.3 μmol) followed by 1 mL of 4 M hydrochloric acid in 1,4-dioxane. The reaction vessel was capped and maintained with stirring at 50° C. for 2 h. The reaction was then concentrated under a stream of compressed air then co-evaporated with 2×4 mL Trace Select grade water under reduced pressure to provide a clear and colourless film. The residue was dissolved in 1 mL of 7:3 of 0.1% trifluoroacetic acid in water:acetonitrile mixture and then purified by preparative C18 HPLC to afford Compound J as clear colourless film (12 mg, 97% purity as determined by HPLC, 46% yield as the TFA salt). An aliquot was analyzed by HPLC elution method 2; retention time=2.4 mins; MS (positive ESI): found m/z=997.1 [M+H]⁺; C₄₈H₆₁N₁₂O₁₂ (calc. 997.5).

### Example 12: Synthesis of N-Hydroxy-2-(7-{[hydroxy(methyl)carbamoyl]methyl}-4,10-bis[(1-hydroxy-6-oxopyridin-2-yl)methyl]-1,4,7,10-tetraazacyclododecan-1-yl)-N-methylacetamide (Compound K)

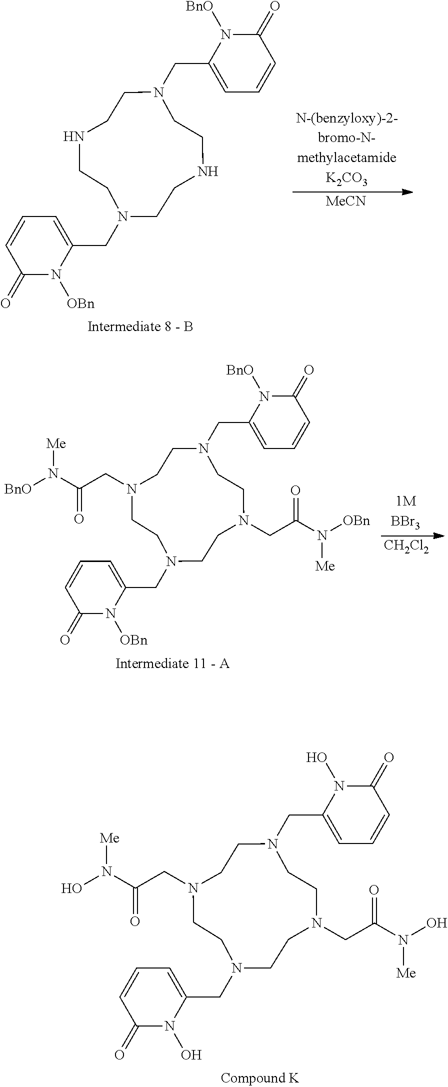

### Step 1: 2-(7-{[Benzyloxy(methyl)carbamoyl]methyl}-4,10-bis({[1-(benzyloxy)-6-oxopyridin-2-yl]methyl})-1,4,7,10-tetraazacyclododecan-1-yl)-N-(benzyloxy)-N-methylacetamide (Intermediate 11-A)

A 20 mL scintillation vial was charged with N-(benzyloxy)-2-bromo-N-methylacetamide (26 mg, 107 μmol), Intermediate 8-B (42 mg, 50.8 μmol) and potassium carbonate (28 mg, 203 μmol) followed by 3 mL of anhydrous acetonitrile and 0.5 g of molecular sieves. The vial headspace was purged with nitrogen and then sealed and heated in an oil bath at 50° C. for 16 h. The reaction was then cooled to room temperature, the solids were removed by filtration and the mother liquor was concentrated to dryness under reduced pressure. The resulting residue was dissolved in 1 mL of 3:7 of 0.1% trifluoroacetic acid in water:acetonitrile mixture and then purified by preparative C18 HPLC to afford Intermediate 11-A as a clear colourless film (35 mg, 98% purity as determined by HPLC, 57% yield as the TFA salt).

### Step 2: N-Hydroxy-2-(7-{[hydroxy(methyl)carbamoyl]methyl}-4,10-bis[(1-hydroxy-6-oxopyridin-2-yl)methyl]-1,4,7,10-tetraazacyclododecan-1-yl)-N-methylacetamide (Compound K)

A 20 mL scintillation vial was charged with Intermediate 11-A (8.5 mg, 7.2 μmol) followed by 1 mL of 1 M boron tribromide in dichloromethane. The reaction vessel was capped and maintained with stirring at 20-25° C. for 3.5 h. The reaction was then concentrated under a stream of compressed air then co-evaporated with 2×4 mL Trace Select grade water then again with 2×4 mL acetonitrile under reduced pressure to provide a clear and colourless film. The film was dissolved in 1 mL of 0.1% trifluoroacetic acid in water and then purified by preparative C18 HPLC to afford Compound K as white chalky film (1 mg, >95% purity as determined by HPLC, 17% yield as the TFA salt). An aliquot was analyzed by HPLC elution method 2; retention time=2.2 mins; MS (positive ESI): found m/z=593.1 [M+H]⁺; C₂₆H₄₁N₈O₈ (calc. 593.3).

### Example 13: Synthesis of 6-({3,9-Bis[(1-hydroxy-6-oxopyridin-2-yl)methyl]-3,6,9,15-tetraazabicyclo[9.3.1]pentadeca-1(15),11,13-trien-6-yl}methyl)-1-hydroxypyridin-2-one (Compound L)

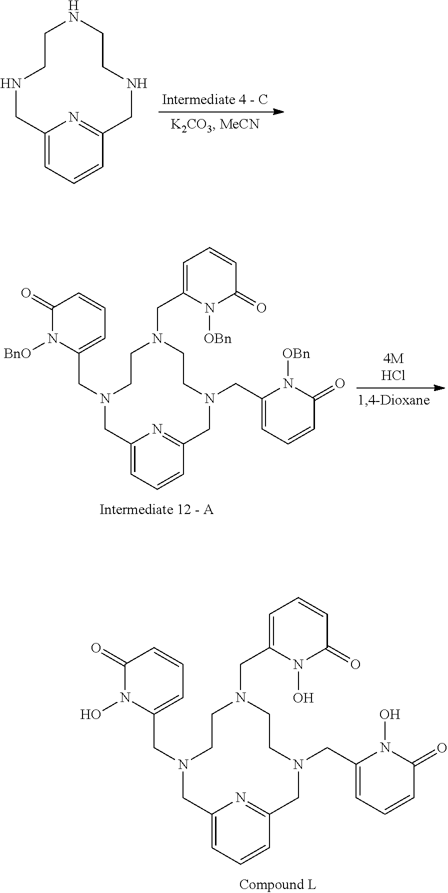 [SMILES: c1cc2nc(c1)CNCCNCCNC2 ; O=c1cccc(CN2CCN(Cc3cccc(=O)n3OCc3ccccc3)Cc3cccc(n3)CN(Cc3cccc(=O)n3OCc3ccccc3)CC2)n1OCc1ccccc1 ; O=c1cccc(CN2CCN(Cc3cccc(=O)n3O)Cc3cccc(n3)CN(Cc3cccc(=O)n3O)CC2)n1O]

### Step 1: 1-(Benzyloxy)-6-{[3,9-bis({[1-(benzyloxy)-6-oxopyridin-2-yl]methyl})-3,6,9,15-tetraazabicyclo[9.3.1]pentadeca-1(15),11,13-trien-6-yl]methyl}pyridin-2-one (Intermediate 12-A)

A 20 mL scintillation vial is charged with 3,6,9,15-tetraazabicyclo[9.3.1]pentadeca-1(15),11,13-triene (30 mg, 145 μmol), Intermediate 4-C (128 mg, 436 μmol) and potassium carbonate (80 mg, 582 μmol) followed by 3 mL of anhydrous acetonitrile and 0.4 g of molecular sieves. The vial headspace is purged with nitrogen and then sealed and heated in an oil bath at 50° C. for 24 h. The reaction is then cooled to room temperature, the solids removed by filtration and the mother liquor is concentrated to dryness under reduced pressure. The resulting residue is dissolved in 2 mL of 1:1 water:acetonitrile mixture and then purified by preparative C18 HPLC to afford Intermediate 12-A in good yield as the TFA salt.

### Step 2: 6-({3,9-Bis[(1-hydroxy-6-oxopyridin-2-yl)methyl]-3,6,9,15-tetraazabicyclo[9.3.1]pentadeca-1(15),11,13-trien-6-yl}methyl)-1-hydroxypyridin-2-one (Compound L)

A 20 mL scintillation vial is charged with Intermediate 12-A 4 M HCl in 1,4-dioxane. The reaction vessel is capped and stirred at 20-25° C. until the reaction is determined to be complete by HPLC analysis. The reaction is then concentrated under a stream of compressed air and co-evaporated with 2×4 mL acetonitrile under reduced pressure. The residue is dissolved in 1 mL of 1:1 0.1% trifluoroacetic acid in water:acetonitrile and then purified by preparative C18 HPLC to afford Compound L in good yield as the TFA salt).

### Example 14: Synthesis of (2R)-4-({2-[1-(1-Hydroxy-6-oxopyridin-2-yl)-N-{2-[2-(2-{2-[3-(2-{2-[3-oxo-3-(2,3,5,6-tetrafluorophenoxy)propoxy]ethoxy}ethoxy)propanamido]ethoxy}ethoxy)ethoxy]ethyl}form amido]ethyl}carbamoyl)-2-[4,7,10-tris(carboxymethyl)-1,4,7,10-tetraazacyclododecan-1-yl]butanoic acid (Compound M) and (2R)-4-{[2-(N-{2-[2-(2-{2-[3-(2-{2-[3-(2,6-dichlorophenoxy)-3-oxopropoxy]ethoxy}ethoxy)propanamido]ethoxy}ethoxy)ethoxy]ethyl}-1-(1-hydroxy-6-oxopyridin-2-yl)formamido)ethyl]carbamoyl}-2-[4,7,10-tris(carboxymethyl)-1,4,7,10-tetraazacyclododecan-1-yl]butanoic acid (Compound N)

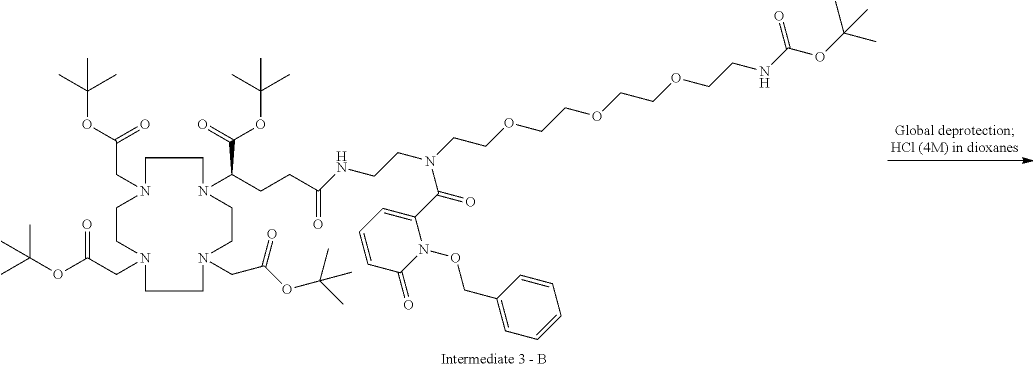 [SMILES: CC(C)(C)OC(=O)CN1CCN(CC(=O)OC(C)(C)C)CCN([C@H](CCC(=O)NCCN(CCOCCOCCOCCNC(=O)OC(C)(C)C)C(=O)c2cccc(=O)n2OCc2ccccc2)C(=O)OC(C)(C)C)CCN(CC(=O)OC(C)(C)C)CC1]

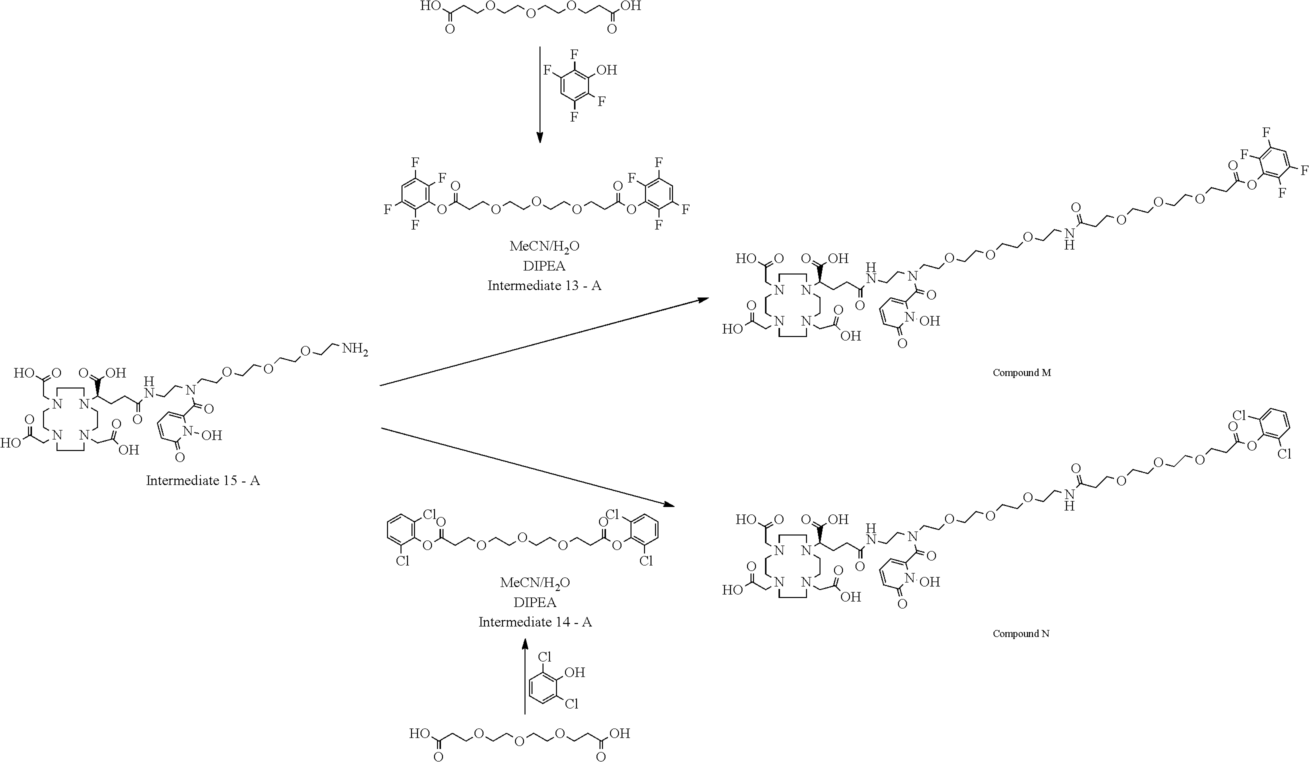

### Step 1: Synthesis of 2,3,5,6-Tetrafluorophenyl 3-(2-{2-[3-oxo-3-(2,3,5,6-tetrafluorophenoxy)propoxy]ethoxy}ethoxy)propanoate (Intermediate 13-A)

To a 20 mL scintillation vial containing 3-{2-[2-(2-carboxyethoxy)ethoxy]ethoxy}propanoic acid (Bis-PEG3-acid, 51 mg, 0.20 mmol) and a stir bar was added a solution of 2,3,5,6-tetraflurophenol (76 mg, 0.43 mmol in 1 mL of anhydrous 1,4-dioxanes). The reaction was then placed in an ice bath to stir and after ˜5 min noticed was no longer fully soluble. Lastly, added N,N′-Dicyclohexylcarbodiimide (DCC, 90 mg, 0.43 mmol) in anhydrous 1,4-dioxanes (0.5 mL) in one portion and then removed the mixture from the ice bath to stir at room temperature for 16 h. The reaction was then monitored by HPLC-MS and worked up by dilution with MeCN (2 mL) and filtration through a fritted filter. The filtered solid was then washed with an additional MeCN (˜5 mL) and the combined filtrate was concentrated under vacuum and purified on a preparative C18 HPLC column to afford Intermediate 13-A (100 mg, 90%, 96% purity) as a clear oil.

### Step 2: Synthesis of 2,6-Dichlorophenyl 3-(2-{2-[3-(2,6-dichlorophenoxy)-3-oxopropoxy]ethoxy}ethoxy)propanoate (Intermediate 14-A)

To a 20 mL scintillation vial containing 3-{2-[2-(2-carboxyethoxy)ethoxy]ethoxy}propanoic acid (bis-PEG3-acid, 250 mg, 0.98 mmol) in 3 mL of anhydrous 1,4-dioxanes was added as stir bar and 2,6-dichlorophenol (365 mg, 2.15 mmol). The clear solution was then placed in an ice bath and stirred for 5 minutes. Lastly, N,N′-dicyclohexylcarbodiimide (DCC, 449 mg, 2.15 mmol) was added in 3 mL of anhydrous 1,4-dioxanes in one portion and then the reaction was removed from the ice bath to stir overnight at room temperature for 6.5 h during which time the reaction progress was monitored by HPLC-MS. Proceeded to add 1 mL of anhydrous DMF which did not fully solubilize the reaction contents and next added HBTU (557 mg, 1.42 mmol) and DIPEA (0.75 mL, 4.31 mmol) and stirred at room temperature for 65 h. The reaction was monitored by HPLC-MS and then worked up by concentration under vacuum to afford a brown oil. The residual DMF remaining was concentrated under an airstream to afford a thick brown oil. The reaction was purified on a preparative C18 HPLC column to afford Intermediate 14-A (319 mg, 60%) as a pale yellow oil. ¹H NMR (600 MHz, CDCl₃)=67.33 (d, J=8.1 Hz, 2H), 7.11 (t, J=8.1 Hz, 2H), 3.90 (t, J=9.0 Hz, 4H), 3.68-3.62 (m, 8H), 2.95 (t, J=6.0 Hz, 4H).

### Step 3: Synthesis of (2R)-4-({2-[N-(2-{2-[2-(2-aminoethoxy)ethoxy]ethoxy}ethyl)-1-(1-hydroxy-6-oxopyridin-2-yl)formamido]ethyl}carbamoyl)-2-[4,7,10-tris(carboxymethyl)-1,4,7,10-tetraazacyclododecan-1-yl]butanoic Acid (Intermediate 15-A)

A scintillation vial containing Intermediate 3-B (34 mg, 16 μmol, 70% purity) was charged with a stir bar and 2 mL of anhydrous HCl (4 M) in dioxanes. The reaction was stirred in a 50° C. oil bath for 4 h and monitored by HPLC-MS. The reaction was then purified on a preparative C18 HPLC column to afford Intermediate 15-A (19 mg, quant) as a clear film as the TFA salt.

### Step 4: Synthesis of (2R)-4-({2-[1-(1-Hydroxy-6-oxopyridin-2-yl)-N-{2-[2-(2-{2-[3-(2-{2-[3-oxo-3-(2,3,5,6 tetrafluorophenoxy)propoxy]ethoxy}ethoxy)propanamido]ethoxy}ethoxy)ethoxy]ethyl}formamido]ethyl}carbamoyl)-2-[4,7,10-tris(carboxymethyl)-1,4,7,10-tetraazacyclododecan-1-yl]butanoic Acid (Compound M)

To a scintillation vial containing Intermediate 15-A (3 mg, 3 μmol) was added H₂O Trace select grade (500 μL), DIPEA (5 μL, 28 μmol) and lastly Intermediate 13-A (5 mg, 8 μmol in 500 μL of MeCN). The resulting solution was stirred at room temperature for 10 min and then quenched by cooling in an ice bath and adding TFA (5 μL). The reaction was then purified on a preparative C18 HPLC column to afford Compound M (4.2 mg, 90%, 93% purity) as a white solid following lyophilization. An aliquot was analyzed by HPLC-MS elution using elution method 2; retention time: 2.91 min; MS (positive ESI): found m/z 1211.1 [M+H]⁺; C₅₁H₇₅F₄N₈O₂₁ (calc. 1211.5).

### Step 5: Synthesis of (2R)-4-{[2-(N-{2-[2-(2-{2-[3-(2-{2-[3-(2,6-Dichlorophenoxy)-3-oxopropoxy]ethoxy}ethoxy)propanamido]ethoxy}ethoxy)ethoxy]ethyl}-1-(1-hydroxy-6-oxopyridin-2-yl)formamido)ethyl]carbamoyl}-2-[4,7,10-tris(carboxymethyl)-1,4,7,10-tetraazacyclododecan-1-yl]butanoic Acid (Compound N)

To a scintillation vial containing Intermediate 15-A (3 mg, 3 μmol) was added H₂O Trace select grade (500 μL), DIPEA (2.5 μL, 14 μmol) and lastly Intermediate 14-A (2 mg, 4 μmol in 500 μL of MeCN). The reaction was stirred at room temperature for 40 min and the reaction progress was monitored by HPLC-MS. The reaction as then stirred in a 50° C. oil bath for 1 h and then additional DIPEA (10 μL) was added followed by an additional 1 h stirring at 50° C. The reaction was concentrated under vacuum and purified on a preparative C18 HPLC column to afford Compound N (3.2 mg, 70%, 90% purity) as an off-white/pale yellow solid following lyophilization. An aliquot was analyzed by HPLC-MS elution using elution method 2; retention time: 2.97 min; MS (positive ESI): found m/z 1207.4 [M+H]⁺; C₅₁H₇₇C₁₂N₈O₂₁ (calc. 1207.5).

### Example 15: Synthesis of (2S)-2-[7-(Carboxymethyl)-4,10-bis[(1-hydroxy-6-oxopyridin-2-yl)methyl]-1,4,7,10-tetraazacyclododecan-1-yl]-5-(2,6-dichlorophenoxy)-5-oxopentanoic Acid (Compound O)

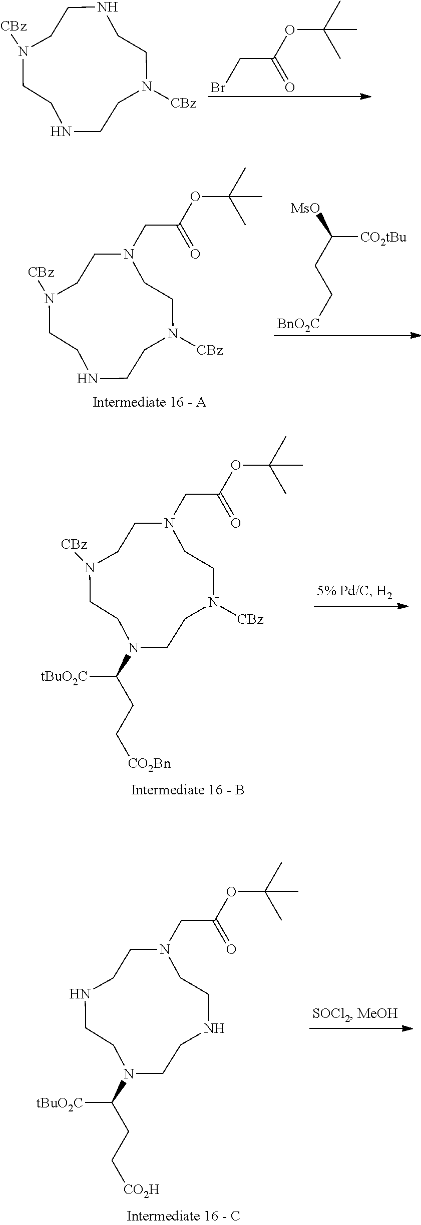

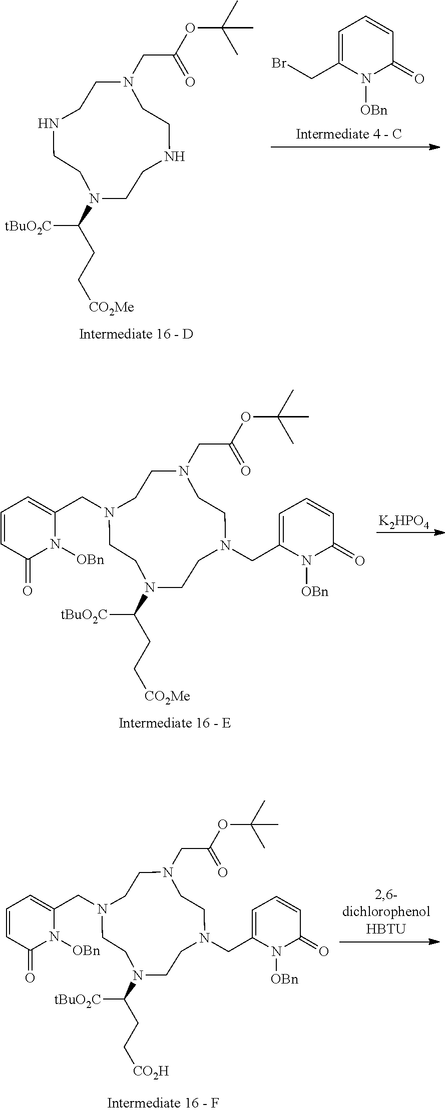 [SMILES: COC(=O)CC[C@@H](C(=O)OC(C)(C)C)N1CCNCCN(CC(=O)OC(C)(C)C)CCNCC1 ; O=c1cccc(CBr)n1OCc1ccccc1 ; COC(=O)CC[C@@H](C(=O)OC(C)(C)C)N1CCN(Cc2cccc(=O)n2OCc2ccccc2)CCN(CC(=O)OC(C)(C)C)CCN(Cc2cccc(=O)n2OCc2ccccc2)CC1 ; "K2HPO4" ; CC(C)(C)OC(=O)CN1CCN(Cc2cccc(=O)n2OCc2ccccc2)CCN([C@@H](CCC(=O)O)C(=O)OC(C)(C)C)CCN(Cc2cccc(=O)n2OCc2ccccc2)CC1]

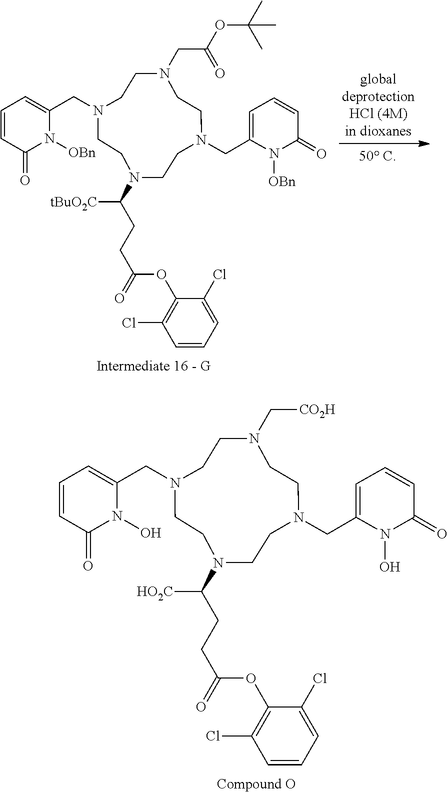 [SMILES: CC(C)(C)OC(=O)CN1CCN(Cc2cccc(=O)n2OCc2ccccc2)CCN([C@@H](CCC(=O)Oc2c(Cl)cccc2Cl)C(=O)OC(C)(C)C)CCN(Cc2cccc(=O)n2OCc2ccccc2)CC1 ; O=C(O)CN1CCN(Cc2cccc(=O)n2O)CCN([C@@H](CCC(=O)Oc2c(Cl)cccc2Cl)C(=O)O)CCN(Cc2cccc(=O)n2O)CC1]

### Step 1: Synthesis of 1,7-Dibenzyl 4-[2-(tert-butoxy)-2-oxoethyl]-1,4,7,10-tetraazacyclododecane-1,7-dicarboxylate (Intermediate 16-A)

To a solution of 1,7-Dibenzyl 1,4,7,10-tetraazacyclododecane-1,7-dicarboxylate dihydrochloride (6.00 g, 11.7 mmol) in MeCN (58 mL) was added DIPEA (8.14 mL, 46.7 mmol) and tert-butyl bromoacetate (1.73 mL, 11.7 mmol). The reaction was stirred in a 60° C. oil bath for 2 h and the reaction progress was monitored by HPLC-MS. The reaction was worked up by concentration under vacuum followed by the addition of Et₂O (100 mL) and KH₂PO₄ (100 mL, 1 M). The resulting mixture was stirred at room temperature for ˜5 min to try to dissolve all contents (some oily pale orange material did not dissolve) and transferred to a separatory funnel. The ether layer was extracted and was found to contain the dialkylated by-product in >80% purity with a minor amount of the desired monoalkylated product. DCM (100 mL) was then used to rinse and dissolve the remaining oily residue in the reaction vessel and was transferred to the aqueous layer from above. The DCM layer was then partitioned and dried over sodium sulfate and concentrated under vacuum to afford a pale-yellow oily residue. The crude was further purified by silica gel chromatography and eluted with the following steps: eluted with 1% MeOH/1% NEt₃/98% DCM (v/v/v) to 2% MeOH/1% NEt₃/97% DCM (v/v/v) respectively. After concentration of the product containing fractions under vacuum obtained Intermediate 16-A (1.53 g, 18%, 76% purity) as a white solid.

### Step 2: Synthesis of 1,7-Dibenzyl 4-[(2S)-5-(benzyloxy)-1-(tert-butoxy)-1,5-dioxopentan-2-yl]-10-[2-(tert-butoxy)-2-oxoethyl]-1,4,7,10-tetraazacyclododecane-1,7-dicarboxylate (Intermediate 16-B)

To a 20 mL scintillation vial with a stir bar was loaded Intermediate 16-A (250 mg, 0.34 mmol), K₂CO₃ (95 mg, 0.69 mmol) and anhydrous acetonitrile (2 mL). Lastly, the 5-benzyl 1-tert-butyl (2R)-2-(methanesulfonyloxy)pentanedioate (191 mg, 0.51 mmol) was added and the mixture was placed in a 80° C. oil bath to stir. After 6 h the reaction progress was monitored by HPLC-MS and found to be only ˜24% conversion so anhydrous DMF (1 mL) was added and the reaction was stirred for an additional 65 h in the 80° C. oil bath. The reaction was worked up by filtration over a fritted filter and the solid was washed with MeCN. The combined filtrate was concentrated under vacuum to afford a light orange oil and purified on a preparative C18 HPLC column to afford Intermediate 16-B (181 mg, 57%, 90% purity) as a clear film.

### Step 3: Synthesis of (4S)-5-(tert-Butoxy)-4-{7-[2-(tert-butoxy)-2-oxoethyl]-1,4,7,10-tetraazacyclododecan-1-yl}-5-oxopentanoic Acid (Intermediate 16-C)

To a 20 mL scintillation vial containing Intermediate 16-B (181 mg, 0.15 mmol) and a stir bar followed by MeOH (3 mL) and then 5% Pd/C (18 mg, 10% wt relative to Intermediate 16-B). The vial was then sealed with a rubber stopper and then the flask was evacuated under vacuum for 1 min while stirring vigorously and then refilled with a H₂ balloon (1 atm) while stirring for 1 min. This cycle of evacuating and then filling was repeated for a total of 3× and then the H₂ balloon was left on the flask and the reaction was allowed to continue to stir at room temperature for 16 h. The reaction progress was monitored by HPLC-MS and then worked up by dilution with methanol (˜3 mL) and then filtered through a 0.2 um GHP syringe filter. The filter was rinsed with an additional MeOH (2×1 mL) and then the combined filtrate was concentrated under vacuum to afford a clear film (134 mg). The crude was then purified on a preparative C18 HPLC column to afford Intermediate 16-C (105 mg, 98%) as a clear film.

### Step 4: Synthesis of 1-tert-Butyl 5-methyl (2S)-2-{7-[2-(tert-butoxy)-2-oxoethyl]-1,4,7,10-tetraazacyclododecan-1-yl}pentanedioate (Intermediate 16-D)

To a 20 mL scintillation vial containing 2 mL anhydrous MeOH and a stir bar in a −5° C. bath (NaCl/ice) was added SOCl₂ (72 μL, 0.99 mmol) dropwise over ˜30 sec. Lastly, a solution of Intermediate 16-C (105 mg, 0.15 mmol) in anhydrous MeOH (1 mL) was added over ˜30 sec and the resulting solution was allowed to continue to stir in the −5° C. bath to ˜0° C. over 1 h. The reaction progress was monitored by HPLC-MS and worked up by concentration under vacuum to afford Intermediate 16-D (81 mg, quant, 97% purity) as a white solid as the HCl salt.

### Step 5: Synthesis of 1-tert-Butyl 5-methyl (2S)-2-[4,10-bis({[1-(benzyloxy)-6-oxopyridin-2-yl]methyl})-7-[2-(tert-butoxy)-2-oxoethyl]-1,4,7,10-tetraazacyclododecan-1-yl]pentanedioate (Intermediate 16-E)

To a 20 mL scintillation vial containing Intermediate 16-D (40 mg, 77 μmol), Intermediate 4-C (70 mg, 0.23 mmol) and a stir bar was added K₂CO₃ (31 mg, 0.23 mmol), and anhydrous MeCN (1 mL). The resulting solution was stirred in a 50° C. oil bath for 65 h and then the reaction progress was monitored by HPLC-MS. The reaction was found to have converted to ˜25% dialkylated product so proceeded to add anhydrous DMF (1 mL) and then stirred the reaction in an oil bath at 80° C. for 4.5 h. The reaction was checked by HPLC-MS and worked up by filtration through a fritted filter. The filtered solid was then washed with additional MeCN (˜5 mL) and the combined filtrate was concentrated under vacuum to obtain a clear film. The crude was then purified on a preparative C18 HPLC column to afford Intermediate 16-E (30 mg, 33%, 94% purity) as a white film.

### Step 6: Synthesis of (4S)-4-[4,10-Bis({[1-(Benzyloxy)-6-oxopyridin-2-yl]methyl})-7-[2-(tert-butoxy)-2-oxoethyl]-1,4,7,10-tetraazacyclododecan-1-yl]-5-(tert-butoxy)-5-oxopentanoic Acid (Intermediate 16-F)

A 20 mL scintillation vial was charged with Intermediate 16-E (30 mg, 26 μmol) followed by a stir bar, THF (0.7 mL), methanol (0.7 mL) and a lithium hydroxide solution freshly prepared (3 mg in 700 μL of H₂O). The reaction was stirred at room temperature for 1 h and the progress was monitored by HPLC-MS. The reaction was worked up by concentration under vacuum and purified on a preparative C18 HPLC column to afford Intermediate 16-F (7.7 mg, 28%) a clear film as the TFA salt.

### Step 7: Synthesis of 1-tert-Butyl 2,6-dichlorophenyl (2S)-2-[4,10-bis({[1-(benzyloxy)-6-oxopyridin-2-yl]methyl})-7-[2-(tert-butoxy)-2-oxoethyl]-1,4,7,10-tetraazacyclododecan-1-yl]pentanedioate (Intermediate 16-G)

To a 20 mL scintillation vial containing Intermediate 16-F (3.8 mg, 3.4 μmol) and a stir bar was added anhydrous MeCN (500 μL), HBTU (2.0 mg, 5.0 μmol; added in 2.0 mg/250 μL anhydrous MeCN) and NEt₃ (4.7 μL, 34 μmol) The resulting solution was stirred for 10 min at room temperature and then a solution of 2,6-dichlorophenol (4 mg, 17 μmol) in MeCN (100 μL) was added and the resulting solution was stirred at room temperature for 2 h. The reaction progress was monitored by HPLC-MS and worked up by concentration under vacuum. The reaction was purified on a preparative C18 HPLC column to afford Intermediate 16-G (4.8 mg, quant.) a clear film as the TFA salt.

### Step 8: Synthesis of (2S)-2-[7-(Carboxymethyl)-4,10-bis[(1-hydroxy-6-oxopyridin-2-yl)methyl]-1,4,7,10-tetraazacyclododecan-1-yl]-5-(2,6-dichlorophenoxy)-5-oxopentanoic acid (Compound O)

To a 1 dram vial containing Intermediate 16-G (2.4 mg, 1.9 μmol) was added a stir bar and 500 μL of anhydrous HCl (4 M) in dioxanes. The reaction was stirred in a 50° C. oil bath for 2 h and the reaction progress was monitored by HPLC-MS. An aliquot was analyzed by HPLC-MS elution using elution method 2; retention time: 2.41 min; MS (positive ESI): found m/z 750.9 [M+H]⁺ and m/z 773.5 [M+Na]⁺; C₃₃H₄₁C₁₂N₆O₁₀ (calc. 751.2) and C₃₃H₄₀Cl₂N₆O₁₀Na (calc. 773.2) respectively. The reaction was then purified on a preparative C18 HPLC column to afford Compound O (1.0 mg, 46%, 85% purity) as a white solid following concentration under vacuum. An aliquot was analyzed by HPLC-MS elution using elution method 2; retention time: 2.39 min; MS (positive ESI): found m/z 774.6 [M+Na]⁺ and m/z 803.6 [M−2H+Fe]⁺; C₃₃H₄₀Cl₂N₆O₁₀Na (calc. 773.2) and C₃₃H₃₈Cl₂FeN₆O₁₀ (calc. 804.1) respectively.

### Example 16: Synthesis of 2,6-Dichlorophenyl 3-[2-(2-{2-[(2-{[4-({1,4,7,10-tetrakis[(1-hydroxy-6-oxopyridin-2-yl)methyl]-1,4,7,10-tetraazacyclododecan-2-yl}methyl)phenyl]carbamoyl}ethyl)carbamoyl]ethoxy}ethoxy)ethoxy]propanoate (Compound P) and 2,6-Dichlorophenyl 1-[(2-{[4-({1,4,7,10-tetrakis[(1-hydroxy-6-oxopyridin-2-yl)methyl]-1,4,7,10-tetraazacyclododecan-2-yl}methyl)phenyl]carbamoyl}ethyl)carbamoyl]-3,6,9,12,15,18,21,24,27,30,33,36-dodecaoxanonatriacontan-39-oate (Compound Q)

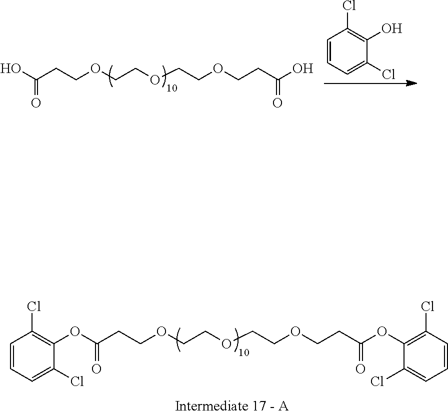

### Step 1: Synthesis of Bis(2,6-dichlorophenyl) 4,7,10,13,16,19,22,25,28,31,34,37-dodecaoxatetracontanedioate (Intermediate 17-A)

To a 20 mL scintillation vial containing Bis-PEG12-acid (250 mg, 0.38 mmol) and a stir bar was added a solution of 2,6-dichlorophenol (192 mg, 1.14 mmol in 3 mL of anhydrous 1,4-dioxanes). The clear solution was then stirred at room temperature and DIPEA (397 μL, 2.27 mmol) was added. The solution was then stirred for 5 min and lastly, HBTU (435 mg, 1.11 mmol) was added in one portion and then the mixture was stirred at room temperature for 3.5 h and was found to have went to completion by HPLC-MS. The reaction was worked up by concentration under vacuum to afford a clear residue and purified on a preparative phenyl HPLC column to afford Intermediate 17-A (234 mg, 65%) as a colourless oil.

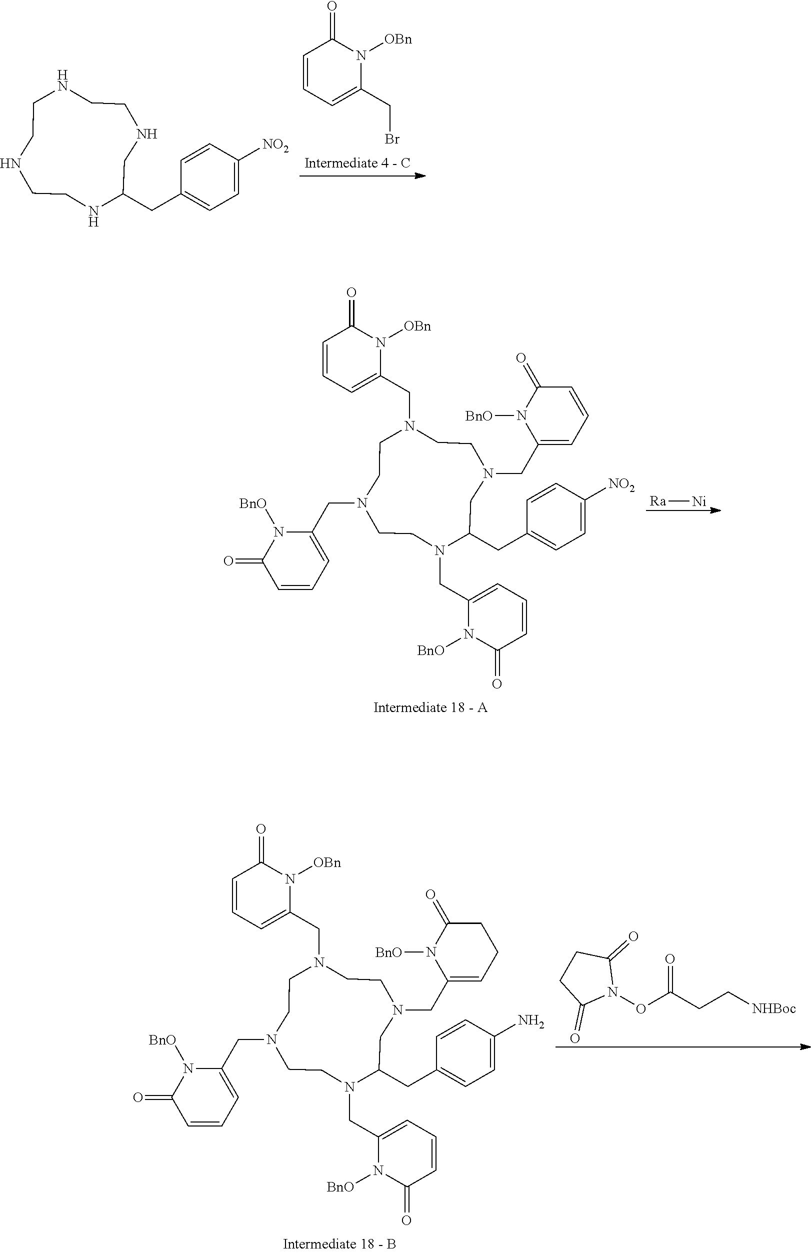

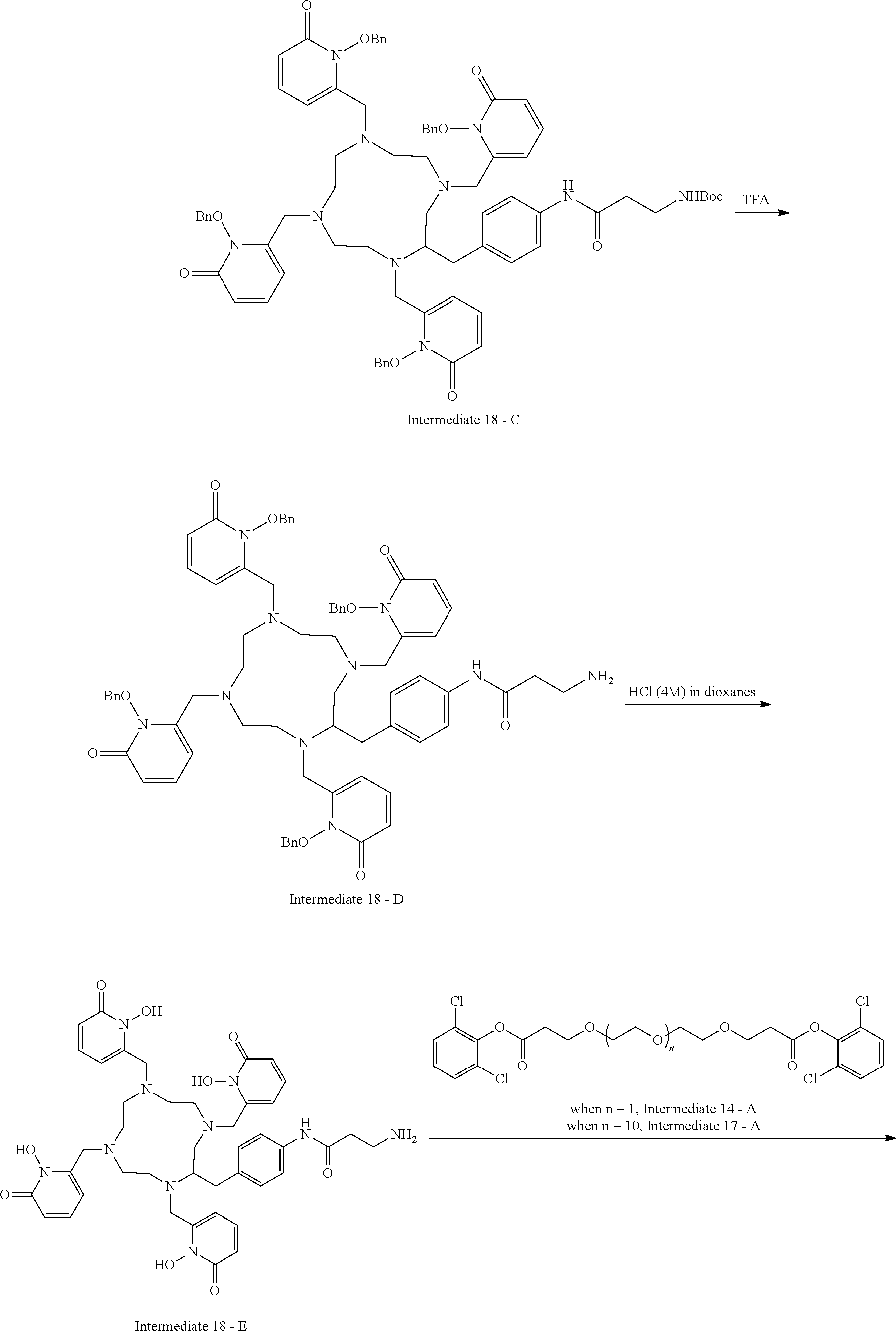

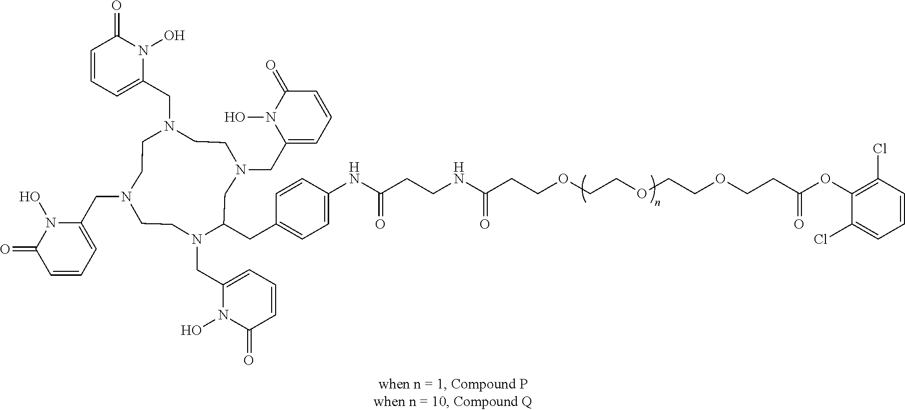

### Step 2: Synthesis of 1-(Benzyloxy)-6-{[4,7,10-tris({[1-(benzyloxy)-6-oxopyridin-2-yl]methyl})-6-[(4-nitrophenyl)methyl]-1,4,7,10-tetraazacyclododecan-1-yl]methyl}pyridin-2-one (Intermediate 18-A)

To a 20 mL scintillation vial containing Intermediate 4-C (112 mg, 0.382 mmol), 2-[(4-nitrophenyl)methyl]-1,4,7,10-tetraazacyclododecane (25 mg, 0.076 mmol) and a stir bar was added K₂CO₃ (63 mg, 0.459 mmol) and anhydrous MeCN (3 mL). The resulting solution was stirred in a 75° C. oil bath for 65 h. The reaction was monitored by HPLC-MS and worked up by filtration through a fritted filter. The filtered solids were washed with MeCN and then the filtrate was concentrated under vacuum and purified on a preparative C18 HPLC column to afford Intermediate 18-A (120 mg, quant.) as a pale yellow film as the TFA salt.

### Step 3: Synthesis of 6-({6-[(4-Aminophenyl)methyl]-4,7,10-tris({[1-(benzyloxy)-6-oxopyridin-2-yl]methyl})-1,4,7,10-tetraazacyclododecan-1-yl}methyl)-1-(benzyloxy)pyridin-2-one (Intermediate 18-B)

A well shaken Ra—Ni 2800 slurry in water (150 μL) was transferred to a 20 mL scintillation vial containing 4 mL of HPLC grade water. The mixture was swirled, allowed to settle and then the water was decanted out (leaving a thin layer on top) and then an additional 4 mL water was used to repeat this wash process. Upon decanting, a 2×4 mL MeOH wash then decant sequence was performed. Lastly, 1 mL of 1:1 THF/MeOH was added, along with a stir bar. Then Intermediate 18-A (20 mg, 0.014 mmol) was added as a solution in 0.5 mL (THF/MeOH, 1:1) and the suspension was then cycled 3× (vacuum for ˜30 seconds then H₂ atmosphere/balloon pressure for ˜ 30 seconds) and the balloon was left on the reaction and it was left to stir at room temperature for 2.5 h. The reaction was monitored by HPLC-MS and worked up by filtering through a 0.2 μm syringe filter. The reaction vial was washed with an additional 2 mL MeOH and filtered through the syringe filter as well. The combined filtrate was then concentrated under vacuum to afford Intermediate 18-B (19.4 mg, 94%) as a pale yellow film.

### Step 4: Synthesis of tert-Butyl N-{2-[(4-{[1,4,7,10-tetrakis({[1-(benzyloxy)-6-oxopyridin-2-yl]methyl})-1,4,7,10-tetraazacyclododecan-2-yl]methyl}phenyl)carbamoyl]ethyl}carbamate (Intermediate 18-C)

To a 20 mL scintillation vial containing Intermediate 18-B (131 mg, 0.077 mmol) was added anhydrous DMF (5 mL) and a stir bar. Next DIPEA (161 μL, 0.93 mmol) was added in one portion followed by DMAP (9.5 mg, 0.077 mmol). The vessel purged with N₂ and then the reaction was stirred at room temperature for 5 min. A freshly dissolved solution of Boc-beta-Ala-OSu (135 mg, 0.463 mmol) in anhydrous DMF (0.5 mL) was added under N₂ atmosphere and then the reaction was stirred in a 50° C. oil bath. After 45 min the reaction progress was monitored by HPLC-MS and primarily starting material along with ˜10% product formation was observed so DMAP (20 mg, 0.164 mmol) and additional Boc-beta-Ala-OSu (135 mg, 0.463 mmol) were added. The reaction was stirred at 50° C. for an additional 18 h. The reaction was worked up by concentration under vacuum and purified on a preparative C18 HPLC column to afford Intermediate 18-C (45 mg, 29%, 76% purity) as a clear film as the TFA salt.

### Step 5: Synthesis of 3-Amino-N-(4-{[1,4,7,10-tetrakis({[1-(benzyloxy)-6-oxopyridin-2-yl]methyl})-1,4,7,10-tetraazacyclododecan-2-yl]methyl}phenyl)propenamide (Intermediate 18-D)

To a 20 mL vial containing Intermediate 18-C (14.5 mg, 0.0090 mmol) was added a stir bar and anhydrous DCM (1 mL) and cooled in an ice bath and then trifluoroacetic acid (2 mL) was added and the reaction was stirred for 30 min at room temperature and the reaction progress was monitored by HPLC-MS. The reaction was worked up by concentration under a nitrogen stream in a fume hood and then further dried under vacuum to afford Intermediate 18-D (22 mg, quant) as a clear film as the TFA salt. This material was used in the subsequent step without further purification.

### Step 6: Synthesis of 3-Amino-N-[4-({1,4,7,10-tetrakis[(1-hydroxy-6-oxopyridin-2-yl)methyl]-1,4,7,10-tetraazacyclododecan-2-yl}methyl)phenyl]propenamide (Intermediate 18-E)

To a 20 mL scintillation vial containing Intermediate 18-D (10 mg, 0.0067 mmol) was added a stir bar and 2 mL of HCl (4 M) in dioxanes. The reaction was stirred in a 50° C. oil bath for 1.5 h and the reaction progress was monitored by HPLC-MS. The reaction was then worked up by concentration under a nitrogen stream and then further dried under vacuum to afford Intermediate 18-E (10 mg, quant) as a pale yellow solid. This material was used in the subsequent step without further purification.

### Step 7: Synthesis of 2,6-Dichlorophenyl 3-[2-(2-{2-[(2-{[4-({1,4,7,10-tetrakis[(1-hydroxy-6-oxopyridin-2-yl)methyl]-1,4,7,10-tetraazacyclododecan-2-yl}methyl)phenyl]carbamoyl}ethyl)carbamoyl]ethoxy}ethoxy)ethoxy]propanoate (Compound P)

To a 20 mL vial containing Intermediate 18-E in ACN/H₂O Trace Select grade (1:1 v/v, 800 μL, ˜8 mg, 0.0053 mmol) was added a stir bar followed by DIPEA (46 μL, 0.26 mmol) and then lastly a solution of Intermediate 14-A (15 mg, 0.027 mmol) in MeCN (400 μL). The reaction was stirred for 1 h at room temperature and then monitored by HPLC-MS. The reaction was worked up by cooling in an ice bath and then adding 50 μL of TFA over ˜30 seconds followed by concentration under vacuum to dryness. The crude was then purified on a preparative C18 HPLC column to afford Compound P (0.7 mg, 7%, ≥81% purity) as a white solid as the TFA salt following lyophilization. An aliquot was analyzed by HPLC-MS elution using elution method 2; retention time: 3.07 min; MS (positive ESI): found m/z 1217.37 [M+H]⁺; C₅₈H₇₁Cl₂N₁₀O₁₅ (calc. 1217.45).

### Step 8: Synthesis of 2,6-Dichlorophenyl 1-[(2-{[4-({1,4,7,10-tetrakis[(1-hydroxy-6-oxopyridin-2-yl)methyl]-1,4,7,10-tetraazacyclododecan-2-yl}methyl)phenyl]carbamoyl}ethyl)carbamoyl]-3,6,9,12,15,18,21,24,27,30,33,36-dodecaoxanonatriacontan-39-oate (Compound Q)

To a 20 mL scintillation vial containing Intermediate 18-E (˜9.0 mg, 0.0080 mmol) in ACN/H₂O Trace Select grade (1:1 v/v, 900 μL/˜1 mg) was added a stir bar followed by DIPEA (70 μL, 0.040 mmol) and then lastly a solution of Intermediate 17-A (37 mg, 0.040 mmol) in MeCN (374 μL). The reaction was stirred for 40 min at room temperature and then monitored by HPLC-MS. The reaction was worked up by cooling in an ice bath and then adding 90 μL of TFA followed by concentration under vacuum to dryness. The crude was then purified on a preparative C18 HPLC column to afford Compound Q (1.2 mg, 6%, ≥68% purity) as a white solid as the TFA salt following lyophilization. An aliquot was analyzed by HPLC-MS elution using elution method 2; retention time: 3.43 min; MS (positive ESI): found m/z 1635.79 [M+Na]⁺; C₇₆H₁₀₆Cl₂N₁₀NaO₂₄ (calc. 1635.67).

### Example 17: Synthesis of 1-Hydroxy-6-({4,7,10-tris[(1-hydroxy-6-oxopyridin-2-yl)methyl]-6-[(4-isothiocyanatophenyl)methyl]-1,4,7,10-tetraazacyclododecan-1-yl}methyl)pyridin-2-one (Compound R)

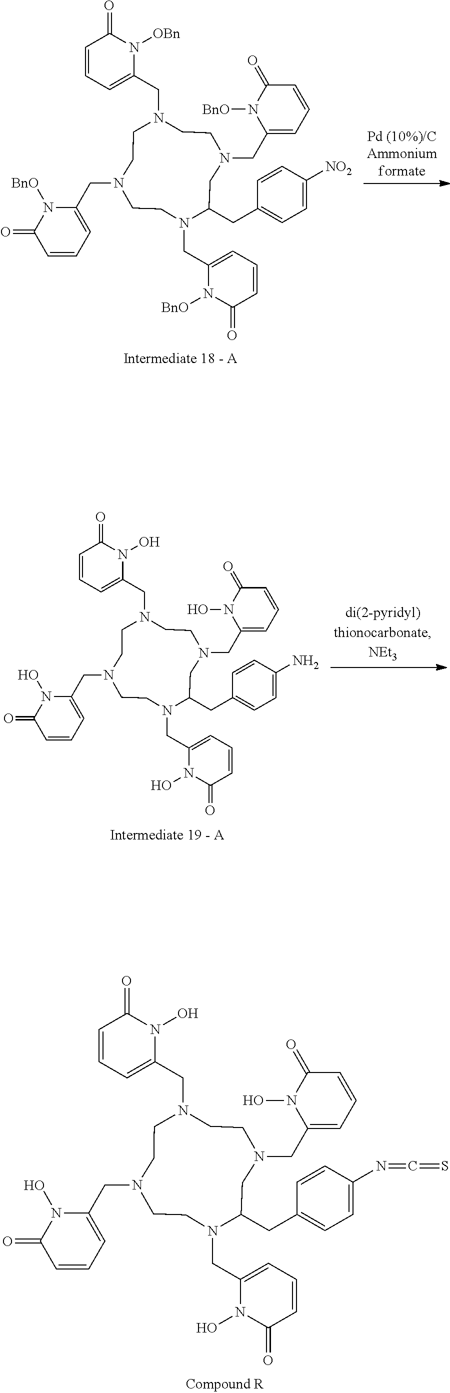 [SMILES: O=c1cccc(CN2CCN(Cc3cccc(=O)n3OCc3ccccc3)CCN(Cc3cccc(=O)n3OCc3ccccc3)C(Cc3ccc([N+](=O)[O-])cc3)CN(Cc3cccc(=O)n3OCc3ccccc3)CC2)n1OCc1ccccc1 ; Nc1ccc(CC2CN(Cc3cccc(=O)n3O)CCN(Cc3cccc(=O)n3O)CCN(Cc3cccc(=O)n3O)CCN2Cc2cccc(=O)n2O)cc1 ; O=c1cccc(CN2CCN(Cc3cccc(=O)n3O)CCN(Cc3cccc(=O)n3O)C(Cc3ccc(N=C=S)cc3)CN(Cc3cccc(=O)n3O)CC2)n1O]

### Step 1: Synthesis of 6-({6-[(4-Aminophenyl)methyl]-4,7,10-tris[(1-hydroxy-6-oxopyridin-2-yl)methyl]-1,4,7,10-tetraazacyclododecan-1-yl}methyl)-1-hydroxypyridin-2-one (Intermediate 19-A)

To a solution of Intermediate 18-A in anhydrous MeOH (1.89 mL) was added Pd (10%)/C (39 mg, 37 μmol) followed by ammonium formate (71 mg, 1131 μmol) and the suspension was stirred at room temperature for 30 min. The reaction progress was monitored by HPLC-MS and then worked up by dilution with MeOH (˜4 mL) and filtration through a 0.2 μm syringe filter (GHP membrane). The reaction vessel was rinsed with MeOH (1 mL) and then passed through the syringe filter as well. The combined filtrate was concentrated under vacuum and then purified on a preparative C18 HPLC column to afford Intermediate 19-A (12.7 mg, 29%, 93% purity) as a white solid.

### Step 2: Synthesis of 1-Hydroxy-6-({4,7,10-tris[(1-hydroxy-6-oxopyridin-2-yl)methyl]-6-[(4-isothiocyanatophenyl)methyl]-1,4,7,10-tetraazacyclododecan-1-yl}methyl)pyridin-2-one (Compound R)

To a solution of Intermediate 19-A (2 mg, 2 μmol) in H₂O Trace Select grade (157 μL)/MeCN (680 μL) was added of NEt₃ (1 μL, 6 μmol) followed by di(2-pyridyl)thionocarbonate (1 mg, 4 μmol). The clear solution turned to a yellow clear color immediately upon addition of the di(2-pyridyl)thionocarbonate and the reaction was left to stir at room temperature for 1 h. The reaction progress was monitored by HPLC-MS and then purified on a preparative C18 HPLC column to afford Compound R (1.6 mg, 62%, ≥81% purity) as a white solid as the TFA salt following lyophilization. An aliquot was analyzed by HPLC-MS elution using elution method 2; retention time: 2.54 min; MS (positive ESI): found m/z 811.9 [M+H]⁺; C₄₀H₄₆N₉O₈S (calc. 812.3).

### Example 18: 4-{[2-(2-{2-[3-(2,6-Dichlorophenoxy)-3-oxopropoxy]ethoxy}ethoxy)ethyl]carbamoyl}-2-{4,7,10-tris[(1-hydroxy-6-oxopyridin-2-yl)methyl]-1,4,7,10-tetraazacyclododecan-1-yl}butanoic (Compound S)

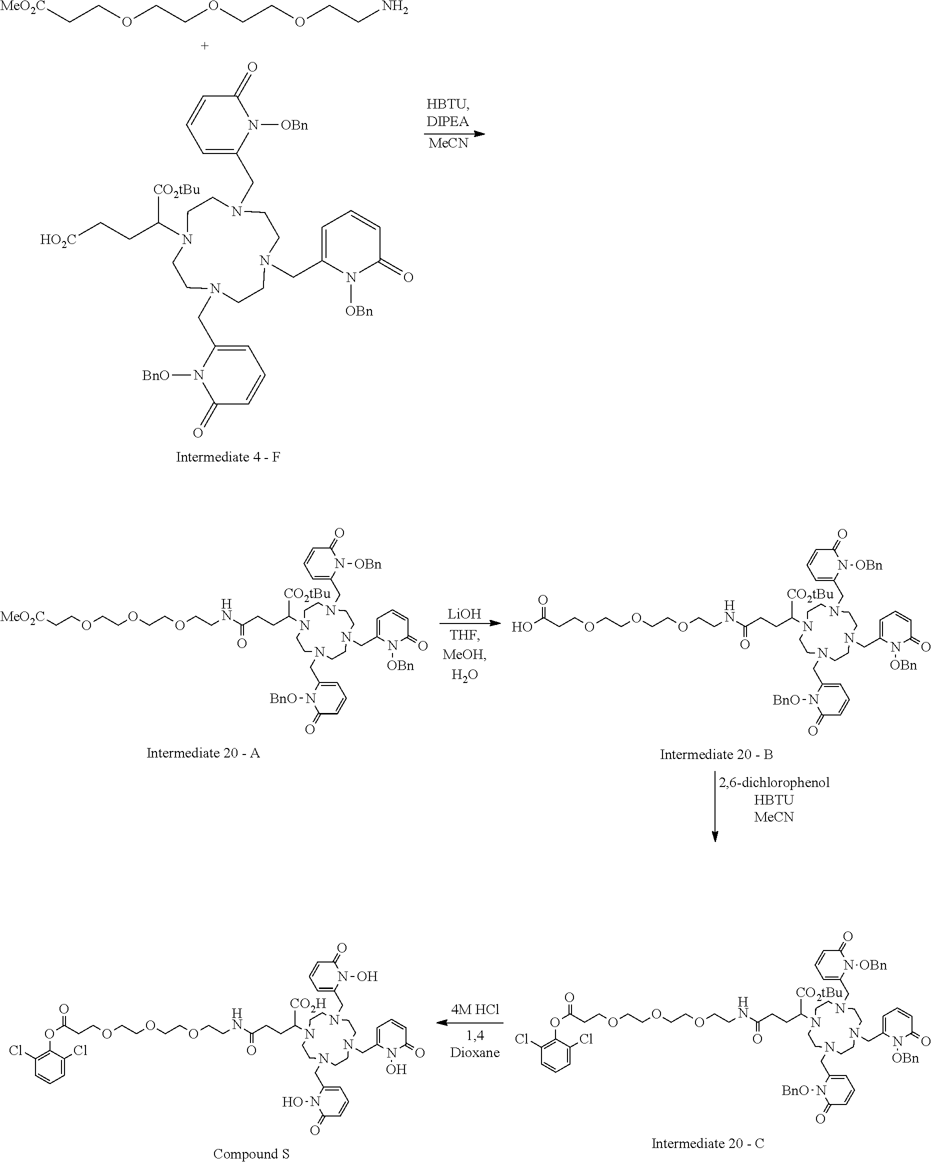

### Step 1: tert-Butyl 4-[(2-{2-[2-(3-methoxy-3-oxopropoxy)ethoxy]ethoxy}ethyl) carbamoyl]-2-[4,7,10-tris({[1-(benzyloxy)-6-oxopyridin-2-yl]methyl})-1,4,7,10-tetraazacyclododecan-1-yl]butanoate (Intermediate 20-A)

A 20 mL scintillation vial was charged with Intermediate 4-F (55 mg, 41 μmol) and HBTU (19 mg, 49.3 μmol) followed by 4 mL of anhydrous acetonitrile and DIPEA (71 μL, 410 μmol) and the mixture was stirred at 20-25° C. for 20 minutes. The HCl salt of amino-PEG3-methyl ester (12 mg, 45.1 μmol) was then added as a solution in 2 mL of anhydrous acetonitrile and the reaction was maintained at 20-25° C. for an additional 1.5 hours. The reaction mixture was then concentrated to dryness under reduced pressure. The resulting residue was dissolved in 1 mL of 1:1 of water:acetonitrile mixture and then purified by preparative C18 HPLC to afford Intermediate 20-A as a clear and colourless film (32 mg, 94% purity as determined by HPLC, 50% yield as the TFA salt).

### Step 2: tert-Butyl 4-{[2-(2-{2-[3-(2,6-dichlorophenoxy)-3-oxopropoxy]ethoxy}ethoxy)ethyl]carbamoyl}-2-[4,7,10-tris({[1-(benzyloxy)-6-oxopyridin-2-yl]methyl})-1,4,7,10-tetraazacyclododecan-1-yl]butanoate (Intermediate 20-B)

A 20 mL scintillation vial was charged with Intermediate 20-A (31.5 mg, 21.8 μmol) followed by 3 mL of a 1:1:1 mixture of water:THF:methanol and then lithium hydroxide (1 mg, 41.8 μmol) and the mixture was maintained at 20-25° C. for 2 h. An additional portion of lithium hydroxide (1 mg, 41.8 μmol) was added and the mixture was maintained at 20-25° C. for 2.5 h. The reaction mixture was then concentrated to dryness under reduced pressure and then dissolved in 1 mL of 1:1 of water:acetonitrile mixture and then purified by preparative C18 HPLC to afford Intermediate 20-B as a clear and colourless oily film (25 mg, 85% purity as determined by HPLC, 68% yield as the TFA salt).

### Step 3: tert-Butyl 4-{[2-(2-{2-[3-(2,6-dichlorophenoxy)-3-oxopropoxy]ethoxy}ethoxy)ethyl]carbamoyl}-2-[4,7,10-tris({[1-(benzyloxy)-6-oxopyridin-2-yl]methyl})-1,4,7,10-tetraazacyclododecan-1-yl]butanoate (Intermediate 20-C)

A 20 mL scintillation vial was charged with Intermediate 20-B (25 mg, 15 μmol) followed by HBTU (16 mg, 41.8 μmol), 3 mL of anhydrous acetonitrile and DIPEA (15 μL, 83.6 μmol) and finally 2,6-dichlorophenol (7 mg, 41.8 μmol) was then added and the mixture was maintained at 20-25° C. for 20 h. Additional portions of HBTU (5 mg, 13.3 μmol) and 2,6-dichlorophenol (5 mg, 30.4 μmol) was then added and the mixture was stirred at 20-25° C. for 4 h. DIPEA (15 μL, 83.6 μmol) and 2,6-dichlorophenol (7 mg, 41.8 μmol) and HBTU (5 mg, 13.3 μmol) were again added and the reaction continued at 20-25° C. for an additional 16 hours. The reaction mixture was then concentrated to dryness under reduced pressure, dissolved in 1 mL of 1:1 of water:acetonitrile mixture and then purified by preparative C18 HPLC. Intermediate 20-C was obtained as a clear and colourless film after concentration (18.7 mg, 97% purity as determined by HPLC, 76% yield as the TFA salt).

### Step 4: 4-{[2-(2-{2-[3-(2,6-Dichlorophenoxy)-3-oxopropoxy]ethoxy}ethoxy)ethyl]carbamoyl}-2-{4,7,10-tris[(1-hydroxy-6-oxopyridin-2-yl)methyl]-1,4,7,10-tetraazacyclododecan-1-yl}butanoic (Compound S)

A 20 mL scintillation vial was charged with Intermediate 20-C (18.7 mg, 11.9 μmol) followed by 1.5 mL of 4 M hydrochloric acid in 1,4-dioxane. The reaction vessel was capped and maintained with stirring at 20-25° C. for 24 h. The reaction was then concentrated under a stream of compressed air then co-evaporated with 2×3 mL acetonitrile. The crude residue was dissolved in 1 mL of 1:1 acetonitrile:0.1% trifluoroacetic acid in water and then purified by preparative C18 HPLC. The fractions determined to contain product were pooled, frozen at −80° C. and lyophilized to afford Compound S as white opaque amorphous solid (7.2 mg, >98% purity as determined by HPLC, 49% yield as the TFA salt). An aliquot was analyzed by HPLC elution method 3; retention time=3.2 mins; MS (positive ESI): found m/z=1019.2 [M+H]⁺; C₄₆H₆₁C₁₂N₈O₁₄ (calc. 1019.4).

### Example 19: 2,6-Dichlorophenyl 3-{2-[2-(3-oxo-3-{4,7,10-tris[(1-hydroxy-6-oxopyridin-2-yl)methyl]-1,4,7,10-tetraazacyclododecan-1-yl}propoxy)ethoxy]ethoxy}propanoate (Compound T)

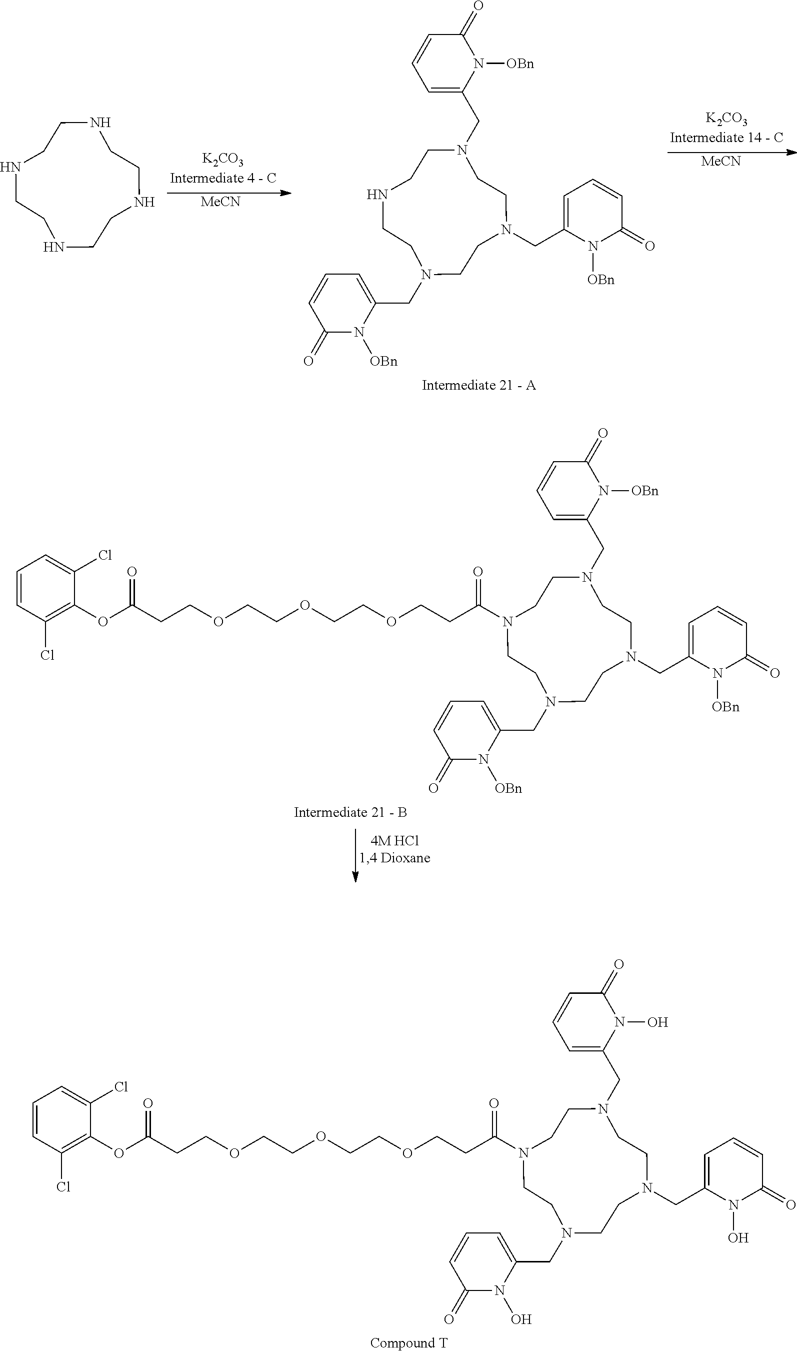 [SMILES: "MeCN" ; "MeCN" ; C1CNCCNCCNCCN1 ; O=c1cccc(CN2CCNCCN(Cc3cccc(=O)n3OCc3ccccc3)CCN(Cc3cccc(=O)n3OCc3ccccc3)CC2)n1OCc1ccccc1 ; O=C(CCOCCOCCOCCC(=O)N1CCN(Cc2cccc(=O)n2OCc2ccccc2)CCN(Cc2cccc(=O)n2OCc2ccccc2)CCN(Cc2cccc(=O)n2OCc2ccccc2)CC1)Oc1c(Cl)cccc1Cl ; O=C(CCOCCOCCOCCC(=O)N1CCN(Cc2cccc(=O)n2O)CCN(Cc2cccc(=O)n2O)CCN(Cc2cccc(=O)n2O)CC1)Oc1c(Cl)cccc1Cl]

### Step 1: 1-(Benzyloxy)-6-{[4,7-bis({[1-(benzyloxy)-6-oxo-1,6-dihydropyridin-2-yl]methyl})-1,4,7,10-tetraazacyclododecan-1-yl]methyl}-1,2-dihydropyridin-2-one (Intermediate 21-A)

A 20 mL scintillation vial was charged with Intermediate 4-C (237 mg, 805 μmol), cyclen (100 mg, 268 μmol) and potassium carbonate (223 mg, 1.61 mmol) followed by 4 mL of anhydrous acetonitrile. The vial headspace was purged with nitrogen and then sealed and heated in an oil bath at 50° C. for 2.5 h. The reaction was then cooled to room temperature, the solids were removed by filtration and the mother liquor was concentrated to dryness under reduced pressure. The residue was dissolved in 2 mL of 1:1 acetonitrile:water mixture and then purified by preparative C18 HPLC to afford Intermediate 21-A as a light yellow oil (91 mg, 90% purity as determined by HPLC, 29% yield as the TFA salt).

### Step 2: 2,6-Dichlorophenyl 3-[2-(2-{3-oxo-3-[4,7,10-tris({[1-(benzyloxy)-6-oxo-1,6-dihydropyridin-2-yl]methyl})-1,4,7,10-tetraazacyclododecan-1-yl]propoxy}ethoxy) ethoxy]propanoate (Intermediate 21-B)

A 20 mL scintillation vial was charged with Intermediate 14-A (12 mg, 22.5 μmol) followed by 1 mL of anhydrous acetonitrile and then potassium carbonate (15 mg, 102 μmol) and finally Intermediate 21-A (24 mg, 20.4 μmol) and the reaction was heated in an oil bath at 85° C. for 23 h. The reaction was cooled to room temperature, the solids were removed by filtration and the mother liquor was concentrated to dryness under reduced pressure. The residue was dissolved in 1 mL of 1:1 acetonitrile:0.1% trifluoroacetic acid in water mixture and then purified by preparative C18 HPLC to afford Intermediate 21-B as an opaque film (7 mg, 90% purity as determined by HPLC, 22% yield as the TFA salt).

### Step 3: 2,6-Dichlorophenyl 3-{2-[2-(3-oxo-3-{4,7,10-tris[(1-hydroxy-6-oxopyridin-2-yl)methyl]-1,4,7,10-tetraazacyclododecan-1-yl}propoxy)ethoxy]ethoxy}propanoate (Compound T)

A 20 mL scintillation vial was charged with Intermediate 21-B (7 mg, 4.9 μmol) followed by 1 mL of 4 M hydrochloric acid in 1,4-dioxane. The reaction vessel was capped and maintained with stirring at 20-25° C. for 22 h. The reaction was then concentrated under a stream of compressed air then co-evaporated with 2×3 mL acetonitrile. The crude residue was dissolved in 1 mL of 1:1 acetonitrile:Trace Select grade water and then purified by preparative C18 HPLC. The product containing fractions were pooled, frozen at −80° C. and lyophilized to afford Compound T as an off-white beige amorphous solid (2.3 mg, >98% purity as determined by HPLC, 41% yield as the TFA salt). An aliquot was analyzed by HPLC elution method 3; retention time=3.1 mins; MS (positive ESI): found m/z=918.1 [M+H]⁺; C₄₂H₅₄C₁₂N₇O₁₂ (calc. 918.3).

### Example 20: 2,6-Dichlorophenyl 3-[2-(2-{2-[({1,4,7,10-tetrakis[(1-hydroxy-6-oxopyridin-2-yl)methyl]-1,4,7,10-tetraazacyclododecan-2-yl}methyl)carbamoyl]ethoxy}ethoxy)ethoxy]propanoate (Compound U)

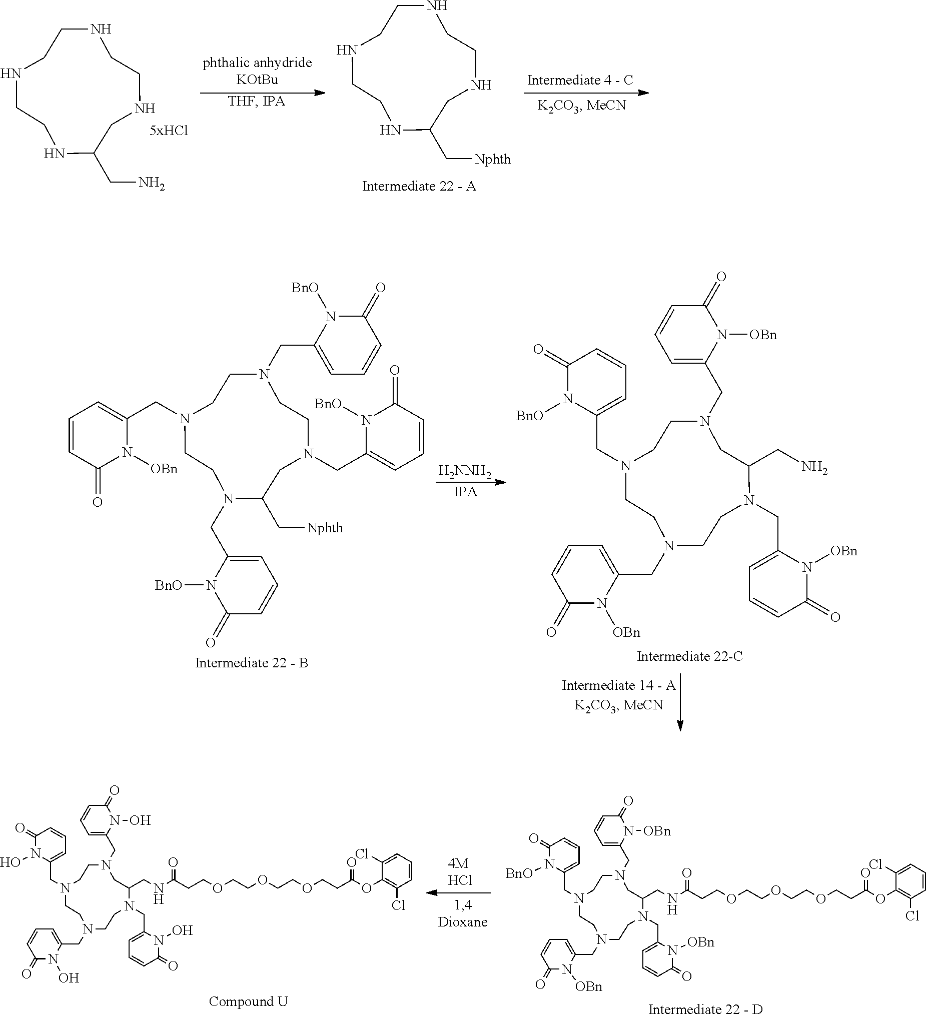

### Step 1: 2-(1,4,7,10-Tetraazacyclododecan-2-ylmethyl)isoindole-1,3-dione (Intermediate 22-A)

A 20 mL scintillation vial was charged with (1,4,7,10-tetraazacyclododecan-2-yl)methanamine 5×HCl (103 mg, 268.5 μmol) followed by 15 mL THF and the suspension was cooled in an ice bath to 0-5° C. Potassium tert-butoxide (150 mg, 1.34 mmol) was then added and the mixture was allowed to slowly warm to 20-25° C. and stirred for 16 h. The resulting mixture was then transferred to a 50 mL 1 neck round bottom flask, concentrated to dryness under reduced pressure, then co-evaporated with 2×10 mL isopropanol. To the dried residue was added 20 mL of isopropanol and then triethylamine (261 μL, 1.88 mmol) and the resulting solution was cooled in an ice bath to 0-5° C. Phthalic anhydride (40 mg, 269 μmol) was then added dropwise as a solution in 1 mL of dichloromethane over 30 mins. The mixture was allowed to warm to room temperature, then a Dean-Stark trap containing isopropanol and a reflux condenser were affixed, and the reaction was set to reflux under a nitrogen atmosphere for 16 h. Reaction completion was confirmed by HPLC-MS and then the reaction mass was concentrated under reduced pressure to a residue, co-evaporated with 2×10 mL acetonitrile and then carried forward without further purification.

### Step 2: 2-{[1,4,7,10-Tetrakis({[1-(benzyloxy)-6-oxopyridin-2-yl]methyl})-1,4,7,10-tetraazacyclododecan-2-yl]methyl}isoindole-1,3-dione (Intermediate 22-B)

A 50 mL 1 neck round bottom flask containing the crude reaction mixture from Step 1 containing Intermediate 22-A (assuming quant. yield; 89 mg, 269 μmol) was charged with Intermediate 4-C (332 mg, 1.13 mmol) and potassium carbonate (223 mg, 1.61 mmol) followed by 10 mL of anhydrous acetonitrile and the reaction was heated in an oil bath at 50° C. for 22 h. The reaction was cooled to room temperature, the solids were removed by filtration and the mother liquor was concentrated to dryness under reduced pressure. The resulting orange-beige foam residue (360 mg) was determined to contain approx. 70% Intermediate 22-B, which was carried forward without further purification.

### Step 3: 6-{[3-(Aminomethyl)-4,7,10-tris({[1-(benzyloxy)-6-oxopyridin-2-yl]methyl})-1,4,7,10-tetraazacyclododecan-1-yl]methyl}-1-(benzyloxy)pyridin-2-one (Intermediate 22-C)

To a 50 mL 1 neck round bottom flask charged with crude Intermediate 22-B (230 mg, 136 μmol, 70% purity) was added 15 mL of isopropanol and amylene (190 μL, 1.8 mmol) then hydrazine-hydrate (190 μL, 3.9 mmol) and the reaction was heated in an oil bath at 95° C. under a nitrogen atmosphere for 16 h. The reaction was then concentrated under reduced pressure and co-evaporated with 2×3 mL acetonitrile to a residue. The crude reaction mixture was dissolved in 1.5 mL of 1:1 acetonitrile:water and then purified by preparative C18 HPLC. Intermediate 22-C was obtained as clear colourless film (44 mg, 95% purity as determined by HPLC, 24% yield as the TFA salt over 3 steps).

### Step 4: 2,6-Dichlorophenyl 3-(2-{2-[2-({[1,4,7,10-tetrakis({[1-(benzyloxy)-6-oxopyridin-2-yl]methyl})-1,4,7,10-tetraazacyclododecan-2-yl]methyl}carbamoyl)ethoxy]ethoxy}ethoxy)propanoate (Intermediate 22-D)

A 20 mL scintillation vial was charged with Intermediate 14-A (30 mg, 56 μmol) followed by 3 mL anhydrous dichloromethane then Intermediate 22-C (24 mg, 18.7 μmol), was added as a solution in 1 mL dichloromethane followed by 1 mL dichloromethane rinse, and then DIPEA (25 μL, 143 μmol) was added and the reaction was maintained at 20-25° C. for 27 h. The reaction mixture was concentrated to dryness under reduced pressure, then co-evaporated with 3×3 mL acetonitrile. The crude residue was then dissolved in 1 mL of 7:5 acetonitrile:water and then purified by preparative C18 HPLC. The product containing fractions were pooled, frozen at −80° C. and lyophilized to afford Intermediate 22-D as white amorphous powder (10 mg, 90% purity as determined by HPLC, 29% yield as the TFA salt).

### Step 5: 2,6-Dichlorophenyl 3-[2-(2-{2-[({1,4,7,10-tetrakis[(1-hydroxy-6-oxopyridin-2-yl)methyl]-1,4,7,10-tetraazacyclododecan-2l}methyl)carbamoyl]ethoxy}ethoxy)ethoxy]propanoate (Compound U)

A 20 mL scintillation vial was charged with Intermediate 22-D (10 mg, 5.4 μmol) followed by 2 mL of 4 M hydrochloric acid in 1,4-dioxane. The reaction vessel was capped and heated to 50° C. for 2.5 h. The reaction was then concentrated under a stream of compressed air then co-evaporated with 2×4 mL acetonitrile. The crude residue was dissolved in 1 mL of 1:1 acetonitrile:0.1% trifluoroacetic acid in Trace Select grade water and then purified by preparative C18 HPLC. The product containing fractions were pooled, frozen at −80° C. and lyophilized to afford Compound U as a fine white amorphous powder (3 mg, 95% purity as determined by HPLC, 40% yield as the TFA salt). An aliquot was analyzed by HPLC elution method 3; retention time=3.0 mins; MS (positive ESI): found m/z=1070.0 [M+H]⁺; C₄₉H₆₂Cl₂N₉O₁₄ (calc. 1070.4).

### Example 21: 2,6-Dichlorophenyl 1-[({1,4,7,10-tetrakis[(1-hydroxy-6-oxopyridin-2-yl)methyl]-1,4,7,10-tetraazacyclododecan-2-yl}methyl)carbamoyl]-3,6,9,12,15,18,21,24,27,30,33,36-dodecaoxanonatriacontan-39-oate (Compound V)

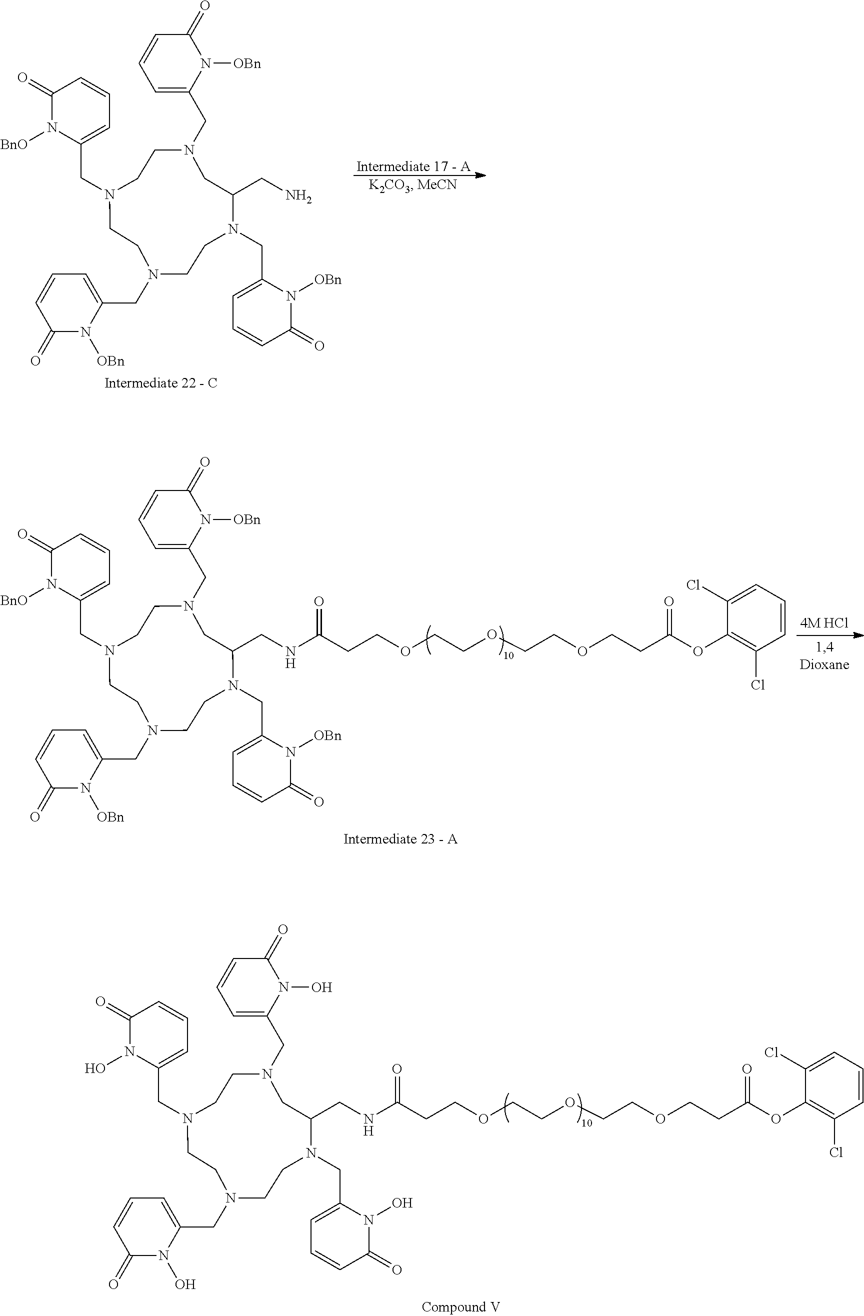

### Step 1: 2,6-Dichlorophenyl 1-({[1,4,7,10-tetrakis({[1-(benzyloxy)-6-oxopyridin-2-yl]methyl})-1,4,7,10-tetraazacyclododecan-2-yl]methyl}carbamoyl)-3,6,9,12,15,18,21,24,27,30,33,36-dodecaoxanonatriacontan-39-oate (Intermediate 23-A)

A 20 mL scintillation vial was charged with Intermediate 17-A (19 mg, 35 μmol) followed by 3 mL anhydrous dichloromethane then Intermediate 22-C (15 mg, 11.7 μmol), was added as a solution in 0.75 mL dichloromethane followed by 0.75 mL dichloromethane rinse, and then DIPEA (32 μL, 187 μmol) was added and the reaction was maintained at 20-25° C. for 24 h. The reaction mixture was concentrated to dryness under reduced pressure, dissolved in 1 mL of 7:5 acetonitrile:water and then purified by preparative C18 HPLC to afford Intermediate 23-A as a clear colourless film (14 mg, >98% purity as determined by HPLC, 58% yield as the TFA salt).

### Step 2: 2,6-Dichlorophenyl 1-[({1,4,7,10-tetrakis[(1-hydroxy-6-oxopyridin-2-yl)methyl]-1,4,7,10-tetraazacyclododecan-2-yl}methyl)carbamoyl]-3,6,9,12,15,18,21,24,27,30,33,36-dodecaoxanonatriacontan-39-oate (Compound V)

A 20 mL scintillation vial was charged with Intermediate 23-A (14 mg, 6.8 μmol) followed by 1.5 mL of 4 M hydrochloric acid in 1,4-dioxane and 1.5 mL of 4 M hydrochloric acid in acetic acid. The reaction vessel was capped and heated to 50° C. for 2.5 h. The reaction was then concentrated under a stream of compressed air then co-evaporated with 2×3 mL acetonitrile. The crude residue was dissolved in 1 mL of 1:1 acetonitrile:0.1% trifluoroacetic acid in Trace Select grade water and then purified by preparative C18 HPLC. The product containing fractions were pooled, frozen at −80° C. and lyophilized to afford Compound V as a yellowish-white amorphous powder (4 mg, 95% purity as determined by HPLC, 33% yield as the TFA salt). An aliquot was analyzed by HPLC elution method 3; retention time=3.4 mins; MS (positive ESI): found m/z=1466.4 [M+H]⁺; C₆₇H₉₈C₁₂N₉O₂₃ (calc. 1466.6).

### Example 22: Synthesis of Antibody Conjugate (Compound W) Using Bifunctional Chelates Compound M and Compound N

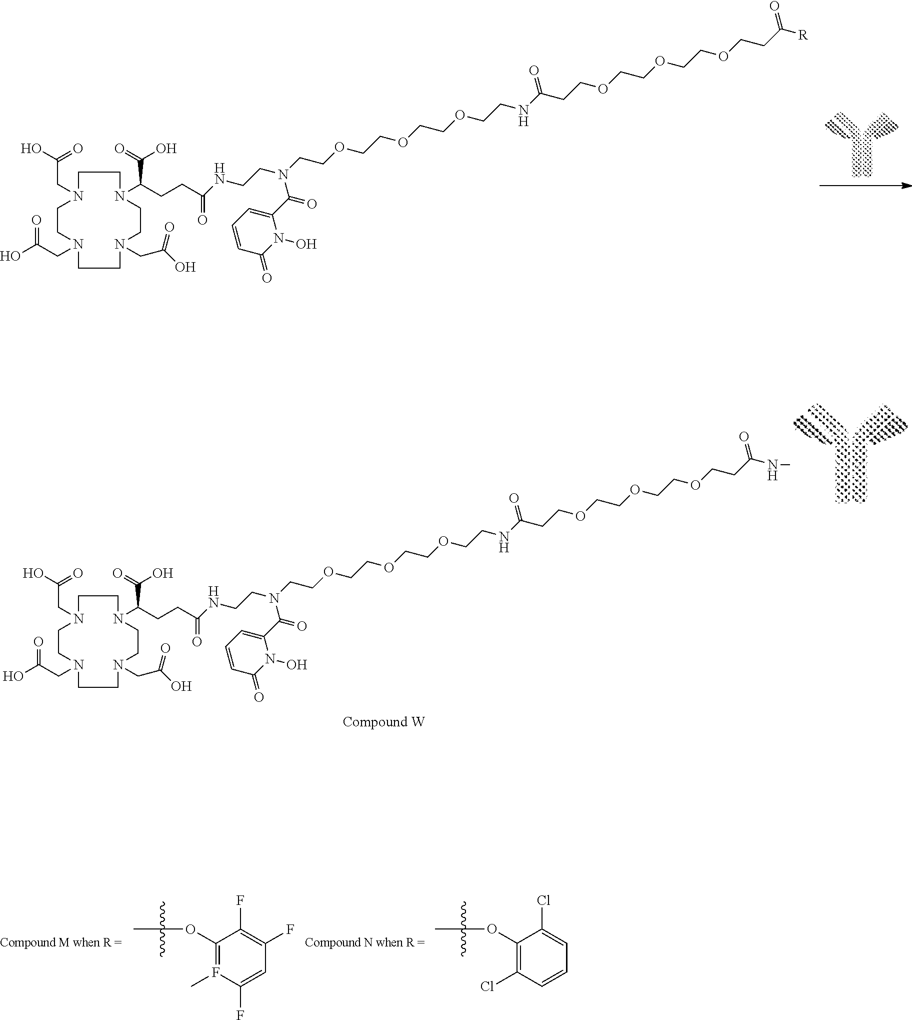

A 500 μL Eppendorf was loaded with an antibody (humanized mAb anti-IGF-1R; 10 nmol, 80.5 uL in a sodium acetate (0.1 M) buffered saline solution with 0.01% Tween 80=SABST) and Na₂CO3 (5 μL, 0.1 M). Compound M or Compound N was added (26 μL, 130 nmol at a c=5 nmol/μL in 0.001 M HCl) followed by Na₂CO₃ (1.2 μL, 0.1 M) to adjust the pH to 8 by pH strip. The reaction was incubated in a 37° C. water bath for 1 h. The reaction was then purified to remove unreacted chelate by G50 column (1 mL housing, elution using SABST) to afford Compound W which was sampled for SEC-HPLC elution method 2 and fitting on a calibration curve for concentration determination (˜78% yield using Compound M and ˜83% yield using Compound N). CAR of 1.1 and 0.44 were determined by MALDI-MS when reacted with Compound M and Compound N respectively.

### Example 23: Synthesis of Antibody Conjugates Compound X and Compound Y

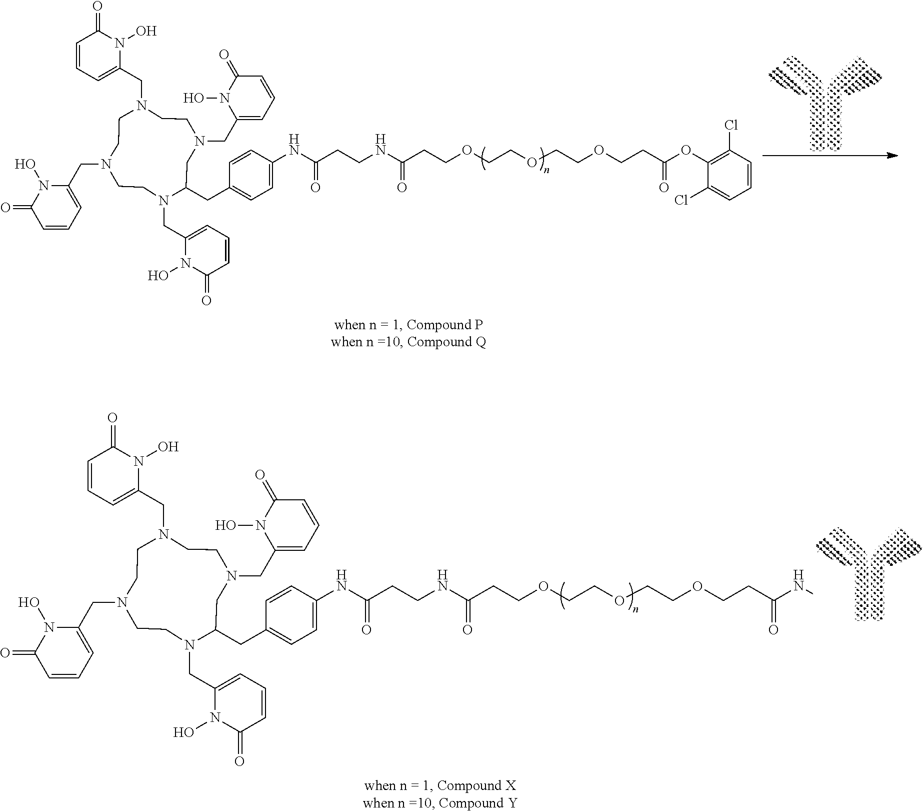

A 1.5 mL Eppendorf was loaded with an antibody (humanized mAb anti-IGF-1R; 9.7 nmol, 1.1 mL in a sodium acetate (0.1 M) buffered saline solution with 0.01% Tween 80=SABST) and sodium bicarbonate buffer (110 μL, 0.1 M). Compound P was added (58.2 μL, 58.2 nmol at a c=1 nmol/μL in 0.001 M HCl). The reaction was incubated at room temperature for 100 min. The reaction was then purified to remove unreacted chelate by G50 column using SABST as eluent to afford Compound X which was sampled by SEC-HPLC elution method 2 and Nano-drop (˜71% yield). A CAR of 0.80 was determined by MALDI-MS. In analogy to the above, a 6 fold excess of Compound Q was reacted with humanized mAb anti-IFG-1R for 120 min at room temperature to afford Compound Y which was sampled by SEC-HPLC elution method 2 and Nano-drop (˜78% yield). A CAR of 0.92 was determined by MALDI-MS.

### Example 24: Radiolabeling of Compound A with ²²⁵Ac

For the ²²⁵Ac radiolabeling of Compound A, the following general conditions were used. A solution of ²²⁵Ac (5 μL, 4 μCi, in 0.001 M HCl) was added to a solution of Compound A (100 μL, 10 nmol) in a sodium acetate (0.1 M, pH 6.5) buffered saline solution with 0.01% Tween 80. The radiolabeling reaction was incubated at 37° C. for 3 hours. The conversion to product was monitored by radioTLC (98.4%; iTLC plate, 1:1:18 NH₄OH/EtOH/H₂O).

### Example 25: Radiolabeling of Compound A with ⁸⁹Zr

For the ⁸⁹Zr radiolabeling of Compound A, the following general conditions were used. A solution of Compound A (10 μL, 50-100 nmol, in 0.001 M HCl) was added to a (4-(2-hydroxyethyl)-1-piperazineethanesulfonic acid; HEPES; 400 μL, 0.5 M) buffer followed by the addition of a solution of ⁸⁹ZrCl₄ (Nucl. Med. Biol. 2009, 36, 729-739) or ⁸⁹Zr(ox)₂ (2-20 μL, 0.5-1.0 mCi). Reactions were heated to 90° C. (1 hour), 60° C. (3 hours) or 37° C. (3 hours), the conversion determined by radioTLC (iTLC plate, 1:1:18 NH₄OH/EtOH/H₂O) and the data summarized in Table 2 below. The resulting products were isolated by radioactive preparative HPLC, concentrated under a stream of air and formulated into a sodium acetate (0.1 M) buffered saline solution with 0.01% Tween 80.

TABLE 2
Conversion Results for the Radiosynthesis of ⁸⁹Zr-Compound A

| | | | Conversion |
|---|---|---|---|
| Temperature (° C.) | Time (h) | ⁸⁹Zr salt | (%) |
| 90 | 1 | ⁸⁹ZrCl₄ | 88 |
| | | ⁸⁹Zr(ox)₂ | 85 |
| 60 | 3 | ⁸⁹ZrCl₄ | 20 |
| | | ⁸⁹Zr(ox)₂ | <5 |
| 37 | 3 | ⁸⁹ZrCl₄ | <5 |
| | | ⁸⁹Zr(ox)₂ | <5 |

### Example 26: Radiolabeling of DOTA with ⁸⁹Zr

A solution of DOTA, S-2-(4-nitrobenzyl)-1,4,7,10-tetraazacyclododecane tetraacetic acid, (Macrocyclics, B-199; 50-100 nmol, 10 μL, in 0.001 M HCl) was added to a HEPES (400 μL, 0.5 M) buffer, followed by the addition of a solution of ⁸⁹ZrCl₄ or ⁸⁹Zr(ox)₂ (2-20 μL, 0.5-1.0 mCi), and the reactions were heated to 90° C. for 1 hour. The conversions were determined by radioTLC (iTLC plate, 1:1:18 NH₄OH/EtOH/H₂O). The ⁸⁹ZrCl₄ resulted in a conversion of 50%, and for the ⁸⁹Zr(ox)₂ the conversion was determined to be 33%. The resulting products were isolated by radioactive preparative HPLC, concentrated under a stream of air and formulated into a sodium acetate (0.1 M) buffered saline solution with 0.01% Tween 80.

### Example 27: Radiolabeling of DFO with ⁸⁹Zr

A solution of DFO, desferrioxamine mesylate salt, (Sigma-Aldrich, D9533; 50-100 nmol, 10 μL, in 0.001 M HCl) was added to a HEPES (400 μL, 0.5 M) buffer, followed by the addition of a solution of ⁸⁹Zr(ox)₂ (2-20 μL, 0.5-1.0 mCi). The reaction was heated to 90° C. for 1 hour, and the conversion determined by radioTLC (>99%; iTLC plate, 0.1 M ethylenediamine tetraacetic acid (EDTA)). The resulting product was isolated by radioactive preparative HPLC, concentrated under a stream of air, and formulated into a sodium acetate (0.1 M) buffered saline solution with 0.01% Tween 80.

### Example 28: Stability of ⁸⁹Zr-Compound A

The stability of the Compound A complex of ⁸⁹Zr was demonstrated using a diethylenetriaminepentaacetic acid (DTPA) challenge experiment, with 25 times molar excess of DTPA added to the HPLC purified ⁸⁹Zr-Compound A, and the results compared to both the ⁸⁹Zr-DOTA and ⁸⁹Zr-DFO analogs. The results summarized in Table 3 below demonstrate that ⁸⁹Zr-Compound A and ⁸⁹Zr-DOTA were stable to the DTPA challenge over 120 hours, and that ⁸⁹Zr-Compound A exhibited superior stability relative to ⁸⁹Zr-DFO under similar conditions.

TABLE 3
Stability of ⁸⁹Zr-Compound A, ⁸⁹Zr-
DOTA, and ⁸⁹Zr-DFO to DTPA Challenge

Percentage of Product (%)

| Compound | 0 hours (initial) | 48 hours | 120 hours |
|---|---|---|---|
| ⁸⁹Zr-Compound A | 96 | 86 | 85 |
| ⁸⁹Zr-DOTA | 99 | 98 | 98 |
| ⁸⁹Zr-DFO | 97 | 61 | 23 |

### Example 29: Radiolabeling and Stability of Compound D, Compound E, and Compound F with ²²⁵Ac

For the ²²⁵Ac radiolabeling of Compound D, Compound E, and Compound F the following general conditions were used. A solution of the Compound (10 μL, 100 nmol, in 0.001 M HCl) was added to a tris(hydroxymethyl)aminomethane (TRIS) buffer (100 μL, 0.1 M). To this was added a solution of ²²⁵Ac (5 μL, 4 μCi, in 0.001 M HCl) and the radiolabeling reaction is incubated at 37° C. for 3 hours. The conversion to product was monitored by radioTLC on ITLC-SG plates that were developed in an appropriate solvent (1:1:18 NH₄OH/EtOH/H₂O or 0.1 M EDTA). The stability of the ²²⁵Ac complex was demonstrated using a DTPA challenge experiment, with 25 times molar excess of DTPA added to the product solution described above. The stability was monitored by radioTLC, and the results of the radiolabeling and stability are summarized in Table 4 below.

TABLE 4
Conversion to Product and DTPA Challenge Stability Results
for ²²⁵Ac-Compound D, ²²⁵Ac-Compound E and ²²⁵Ac-Compound F

| | Conversion to | DTPA Stability |
|---|---|---|
| Compound | Product (%) | (24 hours) |
| ²²⁵Ac-Compound D | 65 | <5 |
| ²²⁵Ac-Compound E | 68 | 47 |
| ²²⁵Ac-Compound F | 87 | 85 |
| ²²⁵Ac-Compound F | 98* | 95 |

*Conditions used: Compound F (10 μL, 100 nmol, in 0.001M HCl) was added to 100 mM sodium acetate buffer pH 6.5, 0.33% NaCl, 0.01% Tween-80. To this was added a solution of ²²⁵Ac (2 μL, 4 μCi, in 0.001M HCl) and the radiolabeling reaction was incubated at 37° C. for 1 h. The conversion and stability was monitored by radioTLC on ITLC-SG plates and developed in 95:5 citrate/MeOH.

### Example 30: Radiolabeling of Compound D, Compound E, and Compound F with ⁸⁹Zr

For the ⁸⁹Zr radiolabeling of Compound D, Compound E and Compound F the following general conditions were used. A solution of the Compound (10 μL, 50-100 nmol, in 0.001 M HCl) was added to a HEPES (400 μL, 0.5 M) buffer. To this was added a solution of ⁸⁹ZrCl₄ or ⁸⁹Zr(ox)₂ (2-20 μL, 0.5-1.0 mCi). Reactions were heated to 90° C. (1 hour), 60° C. (3 hours) or 37° C. (3 hours), the conversion determined by radioTLC (iTLC plate, 0.1 M EDTA) and the data summarized in Tables 5-7 below.

TABLE 5
Conversion Results for the Radiosynthesis of ⁸⁹Zr-Compound D

| | | | Conversion |
|---|---|---|---|
| Temperature (° C.) | Time (h) | ⁸⁹Zr salt | (%) |
| 90 | 1 | ⁸⁹ZrCl₄ | 99 |
| | | ⁸⁹Zr(ox)₂ | 99 |
| 37 | 3 | ⁸⁹ZrCl₄ | 99 |
| | | ⁸⁹Zr(ox)₂ | n/a* |

*not available

TABLE 6
Conversion Results for the Radiosynthesis of ⁸⁹Zr-Compound E

| | | | Conversion |
|---|---|---|---|
| Temperature (° C.) | Time (h) | ⁸⁹Zr salt | (%) |
| 90 | 1 | ⁸⁹ZrCl₄ | 99 |
| | | ⁸⁹Zr(ox)₂ | 99 |
| 37 | 3 | ⁸⁹ZrCl₄ | 99 |
| | | ⁸⁹Zr(ox)₂ | n/a* |

*not available

TABLE 7
Conversion Results for the Radiosynthesis of ⁸⁹Zr-Compound F

| | | | Conversion |
|---|---|---|---|
| Temperature (° C.) | Time (h) | ⁸⁹Zr salt | (%) |
| 90 | 1 | ⁸⁹ZrCl₄ | 99 |
| | | ⁸⁹Zr(ox)₂ | >95 |
| 60 | 3 | ⁸⁹ZrCl₄ | 99 |
| | | ⁸⁹Zr(ox)₂ | >95 |
| 37 | 3 | ⁸⁹ZrCl₄ | 91 |
| | | ⁸⁹Zr(ox)₂ | >95 |

### Example 31: Stability of ⁸⁹Zr-Compound D, ⁸⁹Zr-Compound E, and ⁸⁹Zr-Compound F Towards DTPA

The stability of ⁸⁹Zr-Compound D, ⁸⁹Zr-Compound E, and ⁸⁹Zr-Compound F were demonstrated using a diethylenetriaminepentaacetic acid (DTPA) challenge experiment, with 25 times molar excess of DTPA added to the product solution described above (Example 30). All ⁸⁹Zr radiolabeled compounds were found to be stable toward the DTPA challenge experiment. The stability was monitored by radioTLC, and the results are summarized in Table 8 below.

TABLE 8
DTPA Challenge Stability Results for ⁸⁹Zr-Compound
D, ⁸⁹Zr-Compound E, and ⁸⁹Zr-Compound F

| | Initial Product | Product (%) after 24-hour |
|---|---|---|
| Compound | (%) | DTPA Challenge |
| ⁸⁹Zr-Compound D | 99 | 99 |
| ⁸⁹Zr-Compound E | 99 | 99 |
| ⁸⁹Zr-Compound F | 99 | 99 |

### Example 32: Radiolabeling of Compound D, Compound E, Compound F, Compound H, Compound I, Compound J and Compound K with ⁸⁹Zr and Stability of Complexes Towards EDTA

A solution of ⁸⁹Zr(ox)₂ (4 μL, ˜0.1-0.2 mCi) was neutralized with Na₂CO₃ (2 M, 0.45× volume of Zr-89 solution) then diluted with HEPES (100 μL, 0.5 M, pH=7.1). A solution of the chelate Compound (2-18 μL, 20 nmol, in Trace select grade H₂O) was added and the reaction was heated to 37° C. (30-60 min) and the conversion was determined by radioTLC (iTLC SG plate, 0.1 M EDTA, pH=5). The stability of the ⁸⁹Zr complex was also demonstrated using an EDTA challenge experiment by the addition of 50-500 times molar excess of EDTA to the product solution described above and incubated at room temperature. The stability was monitored by radioTLC, and the results of the radiolabeling and stability are summarized in Table 9 below.

TABLE 9
Radiolabeling with ⁸⁹Zr(ox)₂ in HEPES at 37° C. and
EDTA Challenge Stability Results for ⁸⁹Zr-Compound D,
⁸⁹Zr-Compound E, ⁸⁹Zr-Compound F,
⁸⁹Zr-Compound H, ⁸⁹Zr-Compound I,
⁸⁹Zr-Compound J, ⁸⁹Zr-Compound K

| | | | EDTA Stability |
|---|---|---|---|
| | | Conversion to | (1 day, 50 |
| Compound | Time (min) | Product (%) | equiv) |
| ⁸⁹Zr-Compound D | 60 | >99% | >99% |
| ⁸⁹Zr-Compound E | 60 | >99% | >99% |
| ⁸⁹Zr-Compound F | 60 | >99% | >99% |
| ⁸⁹Zr-Compound H | 30 | >98% | >99%* |
| ⁸⁹Zr-Compound I | 60 | >99% | >99% |
| ⁸⁹Zr-Compound J | 60 | >99% | >99% |
| ⁸⁹Zr-Compound K | 60 | >99% | >99% |

*500 equiv EDTA

### Example 33: Radiolabeling of Compound D, Compound E, Compound F and Compound H with ⁸⁹Z in TRIS Buffer

For the ⁸⁹Zr radiolabeling of Compound D, Compound E, Compound F and Compound H in TRIS buffer the following general conditions were used. A solution of ⁸⁹Zr(ox)₂ (4-10 μL, 0.08-0.4 mCi) was neutralized with Na₂CO₃ (2 M, 0.45× volume of Zr-89 solution) then diluted with TRIS buffer (100-200 μL, 50 mM, pH=7.4). A solution of the chelate Compound (4-36 μL, 20-40 nmol, in Trace Select grade H₂O) was added and the reaction was heated to 37° C. (30 to 60 min) and the conversion was determined by radioTLC (iTLC SG plate, 0.1 M EDTA, pH=5). The data is summarized in Table 10 below.

TABLE 10
Radiolabeling Results in TRIS (0.05M, pH = 7.4)
at 37° C. for ⁸⁹Zr-Compound D, ⁸⁹Zr-Compound E, ⁸⁹Zr-
Compound F and ⁸⁹Zr-Compound H

| | | | Conversion to |
|---|---|---|---|
| Compound | ⁸⁹Zr salt | Time (min) | Product (%) |
| ⁸⁹Zr-Compound D | ⁸⁹Zr(ox)₂ | 60 | >99% |
| ⁸⁹Zr-Compound E | ⁸⁹Zr(ox)₂ | 60 | >99% |
| ⁸⁹Zr-Compound F | ⁸⁹Zr(ox)₂ | 60 | >99% |
| ⁸⁹Zr-Compound H | ⁸⁹Zr(ox)₂ | 30 | >99% |

### Example 34: Radiolabeling of Compound D, Compound E and Compound F with ¹⁷⁷Lu

For the ¹⁷⁷Lu radiolabeling of Compound D, Compound E and Compound F the following general conditions were used. A solution of ¹⁷⁷Lu (1.5 μL, 0.5 mCi, in 0.001M HCl) was added to a solution of the compound (100 μL, 10 nmol) in a sodium acetate (0.1 M, pH 6.5) buffered saline solution with 0.01% Tween 80. The radiolabeling reaction was incubated at 37° C. for 1 hour. The conversion to product was monitored by radioTLC (iTLC plate, 1:1:18 NH₄OH/EtOH/H₂O or 0.1 M EDTA) and the results are summarized in Table 11 below.

TABLE 11
Conversion Results for the Radiosynthesis of ¹⁷⁷Lu-Compound
D, ¹⁷⁷Lu-Compound E, and ¹⁷⁷Lu-Compound F

| | Conversion to |
|---|---|
| Compound | Product (%) |
| ¹⁷⁷Lu-Compound D | 99 |
| ¹⁷⁷Lu-Compound E | 59 |
| ¹⁷⁷Lu-Compound F | 83 |

### Example 35: Radiosynthesis of ⁸⁹Zr-Compound C-Antibody Via 2 Step Labeling

The following general method was used. A solution of DBCO-NHS (BroadPharm, BP-22231; 1000 nmoles in 20 μL DMSO) was added to a solution containing an antibody (humanized mAb anti-IGF-1R; 10.0 nmoles, 250 μL in a sodium acetate (0.1 M) buffered saline solution with 0.01% Tween 80) and a bicarbonate buffer (27 μL). The reaction was incubated at ambient temperature for 1 hour, purified via G-50 resin-packed column eluted with sodium acetate (0.1 M) buffered saline solution with 0.01% Tween 80. The ratio of DBCO to antibody was determined by MALDI-TOF-MS and found to range from 0.1-5.0. The radiolabeling of Compound C with ⁸⁹Zr was as follows; to a solution of ⁸⁹Zr(ox)₂ (1-2 μL, 0.5 mCi) was added a solution of sodium carbonate (0.7 μL, 2 M), which was incubated for 3 minutes. To the mixture was added HEPES (400 μL, 0.5 M) buffer and a solution of Compound C (20 μL, 50 nmoles in 0.001 M HCl) and the reaction was incubated at 90° C. for 1 hour. The solution containing the ⁸⁹Zr-Compound C was then added to the DBCO-antibody (250 μg), and the reaction incubated for 1 hour at ambient temperature. The resulting ⁸⁹Zr-Compound C-Antibody was purified via a Sephadex G-50 resin-packed column eluted with a sodium acetate (0.1 M) buffered saline solution with 0.01% Tween 80. The conversion to ⁸⁹Zr-Compound C-Antibody was monitored by radioTLC (80%; iTLC plate, 0.02 M citrate with 25% methanol) and confirmed by SEC HPLC elution method 1.

### Example 36: Radiolabeling of Antibody Conjugate Compound Y with ⁸⁹Zr and Purification

### By Preparative SEC HPLC

A solution of ⁸⁹Zr(ox)₂ (15-30 μL, 0.8-1.1 mCi) was neutralized with Na₂CO₃ (2 M, 0.45× volume of Zr-89 solution) and then diluted with HEPES (78-140 μL, 0.5 M, pH=7.1). A solution of the antibody conjugate Compound Y (28-200 μL, ˜70-160 μg in a sodium acetate (0.1 M) buffered saline solution with 0.01% Tween 80) was added and the reaction was heated to 37° C. (≤3 h). The reaction was monitored by radioTLC (iTLC SG plate, 0.1 M EDTA, pH=5) and then purified by radioactive preparative SEC HPLC (TOSOH TSK column, 7.8×300 mm, using a phosphate buffer (pH=7) as eluent at a flow=1 mL/min) and reformulated into a sodium acetate (0.1 M) buffered saline solution with 0.01% Tween 80 using a G-25 PD-10 column. The results are summarized in Table 12 and a formulation stability study on ⁸⁹Zr-Compound Y is demonstrated in Table 13 as monitored by radioTLC and SEC HPLC elution method 2 (without the sodium azide).

TABLE 12
Radiosynthesis of ⁸⁹Zr-Compound Y

| | | RCY (%) after | RCP (%) after |
|---|---|---|---|
| | Conversion to | purification and | purification and |
| Compound | Product (%) | reformulation | reformulation |
| ⁸⁹Zr-Compound Y | >83% | 6% | >96% |

TABLE 13
Formulation Stability Study of ⁸⁹Zr-Compound Y at room temperature

| | % Radiochemical | | | % Radiochemical | | | % Monomer | | |
|---|---|---|---|---|---|---|---|---|---|
| | Purity (RCP) by | | | Purity (RCP) by | | | Purity (280 nm) by | | |
| | ITLC | | | HPLC | | | HPLC | | |
| Formulation | D 0 | D 1 | D 4 | D 0 | D 1 | D 4 | D 0 | D 1 | D 4 |
| SABST | ≥96.61 | ≥96.77 | ≥94.81 | ≥95 | ≥95 | ≥95 | 99.50 | 99.39 | 99.41 |
| SABST + | / | ≥97.50 | ≥96.05 | ≥95 | ≥95 | ≥95 | / | 99.48 | 98.73 |
| Gentisic acid | | | | | | | | | |
| Na salt (3.8 mM) | | | | | | | | | |
| SABST + | / | ≥96.42 | ≥93.88 | ≥95 | ≥95 | ≥95 | / | 98.76 | 96.45 |
| Gentisic acid | | | | | | | | | |
| Na salt (16.7 mM) | | | | | | | | | |
| SABST + Na | / | ≥96.74 | ≥95.11 | ≥95 | ≥95 | ≥95 | / | 99.36 | 99.33 |
| Ascorbate (3.8 mM) | | | | | | | | | |
| SABST + Na | / | ≥96.34 | ≥93.10 | ≥95 | ≥95 | ≥95 | / | 99.50 | 99.12 |
| Ascorbate (16.7 mM) | | | | | | | | | |

### Example 37: Bio-Distribution of ⁸⁹Zr-Compound Y-Antibody

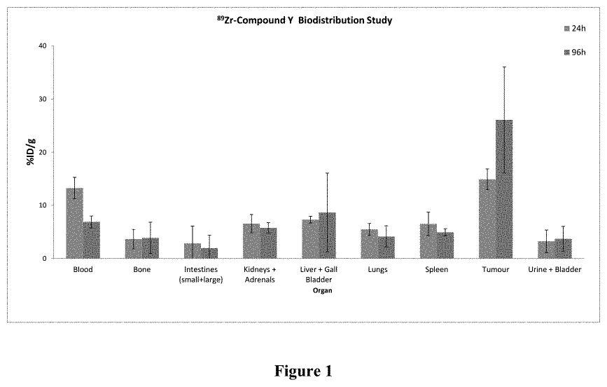

A biodistribution study for ⁸⁹Zr-Compound Y was carried out in female Balb/c nu/nu mice (Charles River) bearing IGF-1R overexpressing Colo-205 (ATCC #CCL-222) colorectal adenocarcinoma tumor xenografts. Tumors were implanted in 7-8 week-old mice by subcutaneous injection of 2×10⁶ viable cells prepared as a suspension in 1:1 (v/v) phosphate buffered saline:Matrigel (Becton-Dickenson). Biodistribution studies were started when tumors reached an initial volume of approximately 200 mm³. Animals were injected intravenously via the lateral tail vein with 200 μL of zirconium-89 labeled immunoconjugate containing 7 μCi of radioactivity conjugated to 3 μg of targeting antibody and formulated in 100 mM sodium acetate buffer pH 6.5, 0.33% NaCl, 0.01% Tween-80, 3.8 mM sodium ascorbate. After selected timepoints (24 and 96 hours) post injection, 3 animals per timepoint were anesthetized with isoflurane, blood was collected by cardiac puncture then the animals were euthanized for organ collection by dissection. Organs and tissue samples were rinsed of blood, blotted of excess moisture and collected into pre-weighed counting tubes. Radiation counts per minute contained in tissue samples were measured using a gamma counter then converted to decay corrected μCi of activity using a calibration standard. Activity measurements and sample weights were used to calculate the percent of injected dose per gram of tissue weight (% ID/g). See FIG. 1.

Results from this biodistribution study indicated that ⁸⁹Zr-Compound Y was capable of delivering Zr-89 isotope to IGF-1R expressing tumors. Tumor uptake (average±standard deviation) was 26.1±10% ID/g after 96 h. Organ uptake was low with an average of less than 9% ID/g across all organs tested. In particular, delivery of Zr-89 to the bone was 3.9±2.9% ID/g.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A compound having the structure of formula (I) below, or a metal complex thereof, or a pharmaceutically acceptable salt thereof: 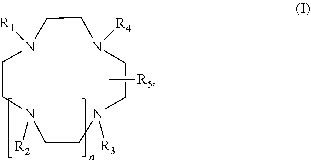
wherein
R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and
n is an integer of 0-3, when n is 0 and R₅ is H, R₁, R₃, and R₄ are not all equal to 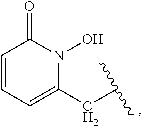
wherein
L is C═O or —CH(R)—, in which R is H, optionally substituted alkyl, optionally substituted heteroalkyl, or -L¹-Z₁-L²-Z₂-B;
U is optionally substituted aryl, optionally substituted heteroaryl, optionally substituted carboxylic acid, or optionally substituted phosphonic acid; or -L-U is -L¹-Z₁-L²-Z₂-B;
at least one of R₁-R₃ has U as optionally substituted heteroaryl;
X is C═O or optionally substituted C₁-C₃ alkylene; and
W is a donating moiety capable of coordinating to a radiometal, wherein the donating moiety is an optionally substituted hydroxypyridinone having the structure selected from the group consisting of 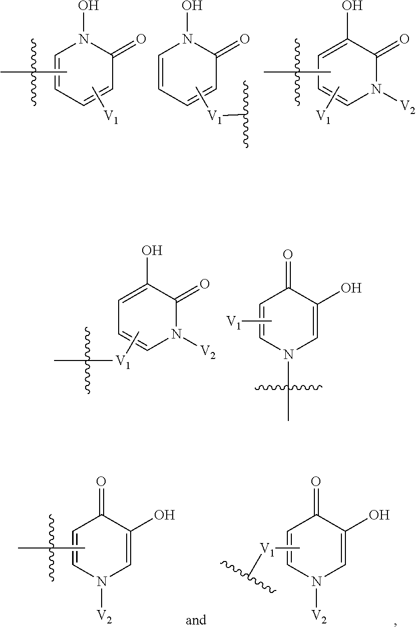
in which V₁ is deleted, fused aryl or heteroaryl, fused carbocycle or heterocycle, alkyl, ether, alcohol, acid, ester, amide, phosphonate or sulfonate; and V₂ is H, alkyl, or acyl,
wherein
L¹ is bond, optionally substituted C₁-C₆ alkylene, or optionally substituted C₁-C₆ heteroalkylene;
Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;
L² is optionally substituted C₁-C₅₀ alkylene, or optionally substituted C₁-C₅₀ heteroalkylene, or C₅-C₂₀ polyethylene glycol;
Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and
B is a therapeutic moiety, a targeting moiety, or cross-linking group.

2. The compound of claim 1, wherein W is

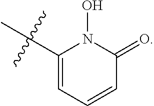 [SMILES: *c1cccc(=O)n1O]

3. The compound of claim 1, wherein X is C₁-C₃ alkylene.

4. The compound of claim 3, wherein W is 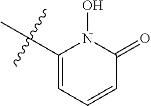 [SMILES: *c1cccc(=O)n1O]
and X is CH₂.

5. The compound of claim 1, wherein n is 1.

6. The compound of claim 5, wherein W is 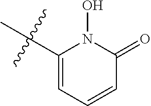 [SMILES: *c1cccc(=O)n1O]
and X is CH₂.

7. The compound of claim 1, wherein R₁, R₂, and R₃ each are, independently, -L-U, in which L is —CH(R)—, R being H.

8. The compound of claim 7, wherein U is optionally substituted heteroaryl or optionally substituted carboxylic acid.

9. The compound of claim 8, wherein U is 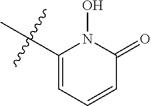 [SMILES: *c1cccc(=O)n1O]
or CO₂H, and at least one of R₁-R₃ has U as

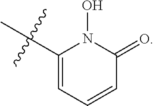 [SMILES: *c1cccc(=O)n1O]

10. The compound of claim 7, wherein at least one of R₁-R₃ has U as

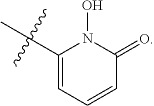 [SMILES: *c1cccc(=O)n1O]

11. The compound of claim 7, wherein each of R₁-R₃ has U as

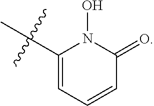 [SMILES: *c1cccc(=O)n1O]

12. The compound of claim 11, wherein W is 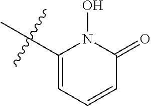 [SMILES: *c1cccc(=O)n1O]
and X is CH₂.

13. The compound of claim 1, wherein R₁, R₂, and R₃ each are, independently, -L-U, in which L is —CH(R)—, R being -L¹-Z₁-L²-Z₂-B and L¹ being

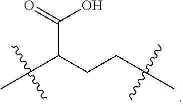 [SMILES: *CCC(*)C(=O)O]

14. The compound of claim 13, wherein L² is C₅-C₂₀ polyethylene glycol and Z₂ is —NR′—(C═O)—R″, R′ being H and R″ being arylene.

15. The compound of claim 13, wherein at least one of R₁-R₃ has U as

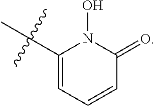 [SMILES: *c1cccc(=O)n1O]

16. The compound of claim 3, wherein each of R₁-R₃ has U as

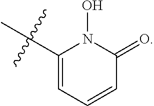 [SMILES: *c1cccc(=O)n1O]

17. The compound of claim 16, wherein W is 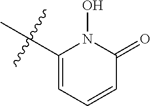 [SMILES: *c1cccc(=O)n1O]
and X is CH₂.

18. The compound of claim 13, wherein B is a therapeutic moiety or targeting moiety.

19. The compound of claim 18, wherein the therapeutic moiety or targeting moiety is an antibody, or an antigen-binding fragment thereof.

20. The compound of claim 19, wherein the antibody, or an antigen-binding fragment thereof, specifically binds IGF-1R.

21. The compound of claim 13, wherein B is a cross-linking group selected from the group consisting of an amino-reactive cross-linking group, a methionine-reactive cross-linking group, and a thiol-reactive cross-linking group.

22. The compound of claim 21, wherein the cross-linking group comprises an activated ester, an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine, in which the activated ester is a hydroxysuccinimide ester, 2,3,5,6-tetrafluorophenol ester, 2,6-dichlorophenol ester or a 4-nitrophenol ester.

23. The compound of claim 22, wherein the cross-linking group is selected from the group consisting of

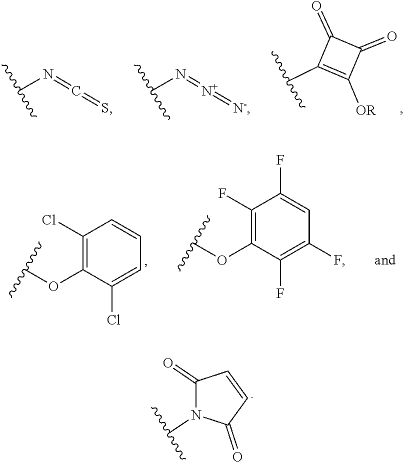

24. The compound of claim 1, wherein the compound comprises a metal complex that contains a metal selected from the group consisting of Bi, Pb, Y, Mn, Cr, Fe, Co, Zn, Ni, In, Ga, Cu, Re, Sm, a lanthanide, and an actinide.

25. The compound of claim 1, wherein the compound comprises a metal complex that contains a radionuclide selected from the group consisting of ⁸⁹Zr, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²³Ra and ²²⁷Th.

26. The compound of claim 25, wherein the radionuclide is ⁸⁹Zr, ¹¹¹In, or ²²⁵Ac.

27. A pharmaceutical composition comprising a compound of claim 1 and a pharmaceutically acceptable carrier.

28. A method of treating an immunoregulatory abnormality in a subject in need thereof, the method comprising administering to said subject a compound of claim 1 in an amount effective for treating said immunoregulatory abnormality.

29. A compound having the structure of formula (I) below, or a metal complex thereof, or a pharmaceutically acceptable salt thereof: 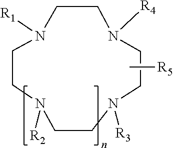
(I),
wherein
R₁, R₂, and R₃ each are, independently, -L-U, R₄ is —X—W, and R₅ is H, -L-U, or —X—W; or R₁, R₂, R₃, and R₄ each are, independently, -L-U, and R₅ is —X—W; and
n is an integer of 0-3,
wherein
L is optionally substituted C₁₋₃ alkylene;
U is optionally substituted carboxylic acid or optionally substituted phosphonic acid;
W is a donating moiety capable of coordinating to a radiometal, in which the donating moiety is an optionally substituted hydroxypyridinone or a moiety selected from the group consisting of 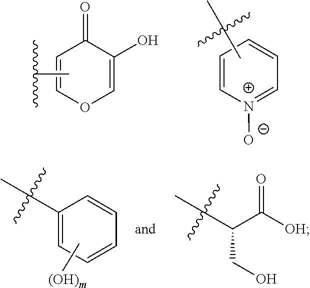
m is an integer of 1-3; and
X is -L¹-Z₁-L²-N(R)—(C═O)—, in which R is H, optionally substituted alkyl, optionally substituted heteroalkyl, or -L³-Z₂-B,
wherein
L¹ and L² each are, independently, bond, optionally substituted C₁-C₆ alkylene, or optionally substituted C₁-C₆ heteroalkylene;
L³ is optionally substituted C₁-C₅₀ alkylene, or optionally substituted C₁-C₅₀ heteroalkylene, or C₅-C₂₀ polyethylene glycol;
Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;
Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and
B is a therapeutic moiety, a targeting moiety, or cross-linking group.

30. The compound of claim 29, wherein W is an optionally substituted hydroxypyridinone, having the structure selected from the group consisting of 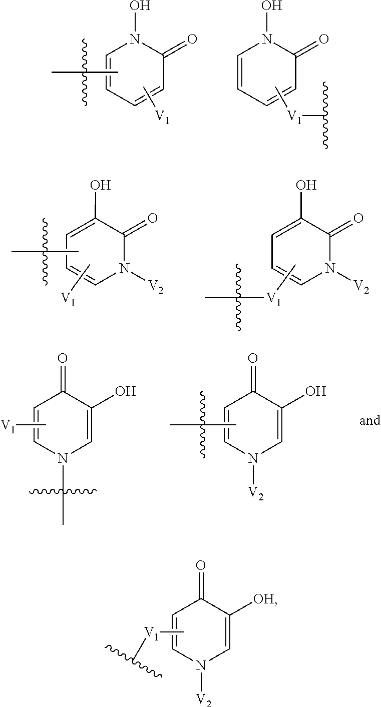
in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

31. The compound of claim 29, wherein R₁, R₂, and R₃ each are, independently, -L-U, in which L is optionally substituted C₁ alkylene and U is —CO₂H.

32. The compound of claim 31, wherein L is CH₂.

33. The compound of claim 29, wherein W is

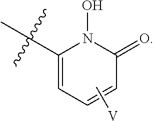

34. The compound of claim 33, wherein W is

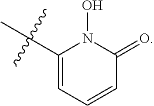 [SMILES: *c1cccc(=O)n1O]

35. The compound of claim 29, wherein n is 1.

36. The compound of claim 35, wherein each of R₁, R₂, and R₃ is -L-U, in which L is CH₂ and U is —CO₂H.

37. The compound of claim 35, wherein W is

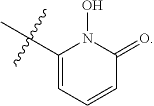 [SMILES: *c1cccc(=O)n1O]

38. The compound of claim 29, wherein X is -L¹-Z₁-L²-N(R)—(C═O)—, in which L¹ is 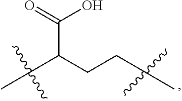 [SMILES: *CCC(*)C(=O)O]
and R is H.

39. The compound of claim 38, wherein each of R₁, R₂, and R₃ is -L-U, in which L is CH₂ and U is —CO₂H.

40. The compound of claim 38, wherein W is

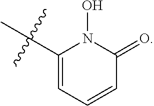 [SMILES: *c1cccc(=O)n1O]

41. The compound of claim 38, wherein each of R₁, R₂, and R₃ is -L-U, in which L is CH₂ and U is —CO₂H; and W is

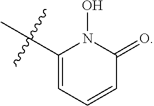 [SMILES: *c1cccc(=O)n1O]

42. The compound of claim 29, wherein X is -L¹-Z₁-L²-N(R)—(C═O)—, in which L¹ is 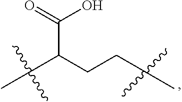 [SMILES: *CCC(*)C(=O)O]
and R is -L³-Z₂-B.

43. The compound of claim 42, wherein L³ is C₅-C₂₀ polyethylene glycol and Z₂ is —NR′—(C═O)—R″, R′ being H and R″ being arylene.

44. The compound of claim 42, wherein each of R₁, R₂, and R₃ is -L-U, in which L is CH₂ and U is —CO₂H.

45. The compound of claim 42, wherein W is

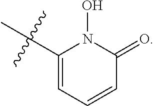 [SMILES: *c1cccc(=O)n1O]

46. The compound of claim 45, wherein each of R₁, R₂, and R₃ is -L-U, in which L is CH₂ and U is —CO₂H; L³ is C₅-C₂₀ polyethylene glycol; and Z₂ is —NR′—(C═O)—R″, R′ being H and R″ being arylene.

47. The compound of claim 42, wherein B is a therapeutic moiety or targeting moiety.

48. The compound of claim 47, wherein the therapeutic moiety or targeting moiety is an antibody, or an antigen-binding fragment thereof.

49. The compound of claim 48, wherein the antibody, or an antigen-binding fragment thereof, specifically binds insulin-like growth factor-1 receptor (IGF-1R).

50. The compound of claim 42, wherein B is a cross-linking group selected from the group consisting of an amino-reactive cross-linking group, a methionine-reactive cross-linking group, and a thiol-reactive cross-linking group.

51. The compound of claim 50, wherein the cross-linking group comprises an activated ester, an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine, in which the activated ester is a hydroxysuccinimide ester, 2,3,5,6-tetrafluorophenol ester, 2,6-dichlorophenol ester or a 4-nitrophenol ester.

52. The compound of claim 51, wherein the cross-linking group is selected from the group consisting of:

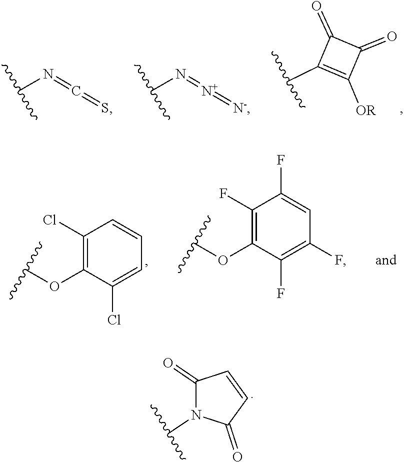

53. The compound of claim 29, wherein the compound comprises a metal complex that contains a metal selected from the group consisting of Bi, Pb, Y, Mn, Cr, Fe, Co, Zn, Ni, In, Ga, Cu, Re, Sm, a lanthanide, and an actinide.

54. The compound of claim 29, wherein the compound comprises a metal complex that contains a radionuclide selected from the group consisting of ⁸⁹Zr, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²³Ra and ²²⁷Th.

55. The compound of claim 54, wherein the radionuclide is ⁸⁹Zr, ¹¹¹In, or ²²⁵Ac.

56. A pharmaceutical composition comprising a compound of claim 29 and a pharmaceutically acceptable carrier.

57. A method of treating an immunoregulatory abnormality in a subject in need thereof, the method comprising administering to said subject a compound of claim 29 in an amount effective for treating said immunoregulatory abnormality.

58. A compound having the structure of formula (II) below, or a metal complex thereof, or a pharmaceutically acceptable salt thereof: 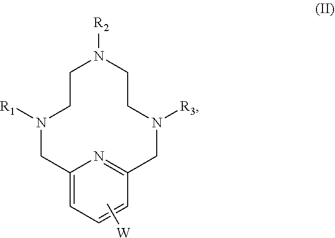
wherein
R₁, R₂, and R₃ each are, independently, -L-U, and W is H or -L¹-Z₁-L²-Z₂-B,
wherein
L is C═O or —CH(R)—, in which R is H, optionally substituted alkyl, optionally substituted heteroalkyl, or -L¹-Z₁-L²-Z₂-B;
U is optionally substituted aryl, optionally substituted heteroaryl, optionally substituted carboxylic acid, or optionally substituted phosphonic acid; or -L-U is -L¹-Z₁-L²-Z₂-B;
at least one of R₁-R₃ has U as optionally substituted heteroaryl;
wherein
L¹ is bond, optionally substituted C₁-C₆ alkylene, or optionally substituted C₁-C₆ heteroalkylene;
Z₁ is bond, C═O(NR⁴), C═S(NR⁴), OC═O(NR⁴), NR⁴C═O(O), NR⁴C═O(NR⁴), —CH₂PhC═O(NR⁴), —CH₂Ph(NR⁴)C═O, or —CH₂Ph(NH)C═S(NR⁴), each R⁴ independently being H, optionally substituted C₁-C₆ alkyl, optionally substituted C₁-C₆ heteroalkyl, or optionally substituted aryl or heteroaryl;
L² is optionally substituted C₁-C₅₀ alkylene, or optionally substituted C₁-C₅₀ heteroalkylene, or C₅-C₂₀ polyethylene glycol;
Z₂ is C═O, —NR′—(C═O)—, or —NR′—(C═O)—R″, R′ being H or C₁-C₆ alkyl and R″ being C₁-C₂₀ alkylene, C₂-C₂₀ heteroalkylene, or arylene; and
B is a therapeutic moiety, a targeting moiety, or cross-linking group.

59. The compound of claim 58, wherein U is a donating moiety capable of coordinating to a radiometal, wherein the donating moiety is an optionally substituted hydroxypyridinone having the structure selected from the group consisting of 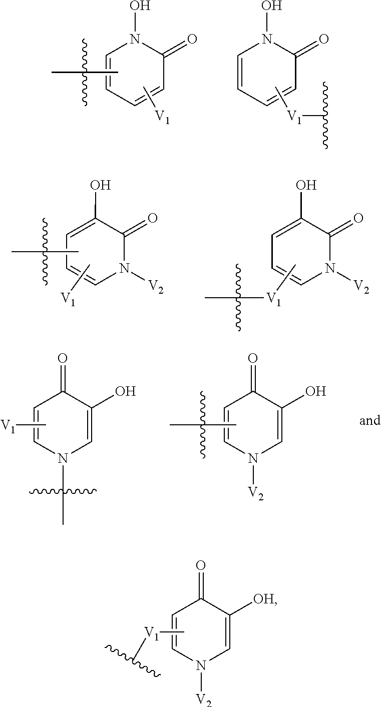
in which V₁ is deleted, fused aryl or heteroaryl, fused carbocycle or heterocycle, alkyl, ether, alcohol, acid, ester, amide, phosphonate or sulfonate; and V₂ is H, alkyl, or acyl.

60. A pharmaceutical composition comprising a compound of claim 58 and a pharmaceutically acceptable carrier.

61. A method of treating an immunoregulatory abnormality in a subject in need thereof, the method comprising administering to said subject a compound of claim 58 in an amount effective for treating said immunoregulatory abnormality.
